(19) United States
(12) Patent Application Publication
(10) Pub. No.: US 2026/0207573 A1
(43) Pub. Date: Jul. 23, 2026

(54) EFFECT OF RIVOCERANIB ON CYTOCHROME P450 ENZYME SUBSTRATES

(71) Applicant: ELEVAR THERAPEUTICS, INC., Fort Lee, NJ (US)

(72) Inventor: Seong Hoon Jang, Gaithersburg, MD (US)

(21) Appl. No.: 19/141,913

(22) PCT Filed: Dec. 20, 2023
(86) PCT No.: PCT/US2023/085156

§ 371 Date: Jun. 20, 2025

Related U.S. Application Data
(60) Provisional application No. 63/434,349, filed on Dec. 21, 2022.
(60) Provisional application No. 63/456,306, filed on Mar. 31, 2023.

(51) Int. Cl. A61K 31/444; A61K 31/485

(52) U.S. Cl. CPC A61K 31/444; A61K 31/485

(57) ABSTRACT

The present disclosure provides methods of treating cancer in a patient with a therapeutically effective amount of rivoceranib while managing drug-drug interactions with a co-dosed second therapeutic agent that is a substrate of cytochrome P-450 enzymes selected from any one or more of CYP3A4/A5, CYP2D6, CYP2C19, CYP2C9, or CYP1A2.

### FIELD OF THE DISCLOSURE

The present disclosure relates to methods of treatment of cancer with a small molecule tyrosine kinase inhibitor in a manner that manages drug-drug interactions.

### BACKGROUND OF THE DISCLOSURE

Cancer remains one of the most deadly threats to human health, affecting over 1 million new patients each year in the United States. Although there have been significant advances in the medical treatment of certain cancers, current methods of treatment remain relatively non-selective: surgery removes the diseased tissue; radiotherapy shrinks solid tumors; and chemotherapy kills rapidly dividing cells. A universally accepted approach to treating rapidly dividing cells in solid tumors is “anti-angiogenesis therapy” first put forward by Dr. Folkman in early 1970s.

Angiogenesis plays an important role in malignant tumor growth and metastasis. When tumors grow beyond 1 mm³, angiogenesis, or generation of vascular arborizations by budding from existing vessels, is necessary to provide enough blood for the survival of tumor cells. The growth speed and tendency of metastasis of tumors are associated with the level of neovascularization factors and the quantity of nascent microvessels. The resulting new blood vessels “feed” growing tumors with oxygen and nutrients, allowing the tumor to enlarge and the cancer cells to invade nearby tissue, to move throughout the body, and to form new colonies of cancer cells, called metastases.

Inhibition of angiogenesis can be accomplished in several ways. One approach that has proven to be clinically successful is targeting Vascular Endothelial Growth Factor (VEGF), which is a mediator of angiogenesis in cancer. Angiogenesis inhibitors bind to VEGF and/or its receptor as well as to other receptors on the surface of endothelial cells or to other proteins in the downstream signaling pathways, blocking their activities.

To date, the U.S. Food and Drug Administration (FDA) has approved a number of angiogenesis inhibitors to treat cancer. Most of these are targeted therapies developed to specifically target VEGF, its receptor, or other processes involved in angiogenesis. Each of these have their own unique therapeutic window, i.e., efficacy versus side-effects, and use.

Rivoceranib (also known as YN968D1, developed in China as apatinib and marketed as Aitan®) is an orally administered small molecule tyrosine kinase inhibitor with selectivity towards the VEGFR-2/kinase insert domain receptor. Rivoceranib has received approval in China, for treatment of advanced gastric cancer, and has received orphan medicinal product designation for the treatment of gastric cancer from Europe, the FDA, and the MFDS in South Korea. According to a recent review (see L. J. Scott, “Apatinib: A Review in Advanced Gastric Cancer and Other Advanced Cancers,” Drugs, 2018, 78(7), 747-758), “further clinical experience and long-term pharmacovigilance data are required to more definitively establish the efficacy and safety profile of apatinib, including its use in combination with other chemotherapy agents . . . .”

Combination therapy, which combines the use of two or more therapeutic agents, is a cornerstone of cancer therapy. The amalgamation of anti-cancer drugs enhances efficacy compared to the mono-therapy approach, because it targets key pathways in a characteristically synergistic or additive manner. It has been demonstrated that combination therapy can produce a more effective treatment response in fewer cycles, and therefore this treatment modality reduces the incidence of resistance (see, e.g., Hanahan et al, J Clin Invest. 2000:105:1045-7).

However, drugs taken together often result in a drug-drug interaction (DDI), where one drug's pharmacokinetics (PK) is altered by the concomitant administration of the other drug. Alteration in PK can happen, for example, when one drug is metabolized by one or more cytochrome P450 (CYP) enzymes and the other drug inhibits or induces one or more of the same CYP enzyme. Inhibition of CYP enzymes is considered a principal mechanism for metabolism-based DDI and usually involves competition of two drugs for the same CYP binding site (Kumar et al, Curr. Med. Chem. 2012; 19:3605-3621). CYP inhibition can result in either drug accumulation or decreased drug metabolism, leading to possible clinical toxicity or enhancement of pharmacological effects (Manikandan P., et al. Curr. Drug Targets. 2018, 19:38-54).

Changes in pharmacokinetic parameters such as AUC, Cₘₐₓ, and t_{1/2} of a drug taken in combination with rivoceranib due to modulation of CYP enzyme activity by rivoceranib can lead to many problems, including complications for physicians in prescribing, underdosing, or overdosing of the drug. Thus, the need exists for new dosing regimens for combinations of rivoceranib and other drugs that address and manage potential DDI issues.

### SUMMARY OF THE DISCLOSURE

The present disclosure provides, inter alia, methods of treating cancer in a patient with a therapeutically effective amount of rivoceranib while managing drug-drug interactions (DDI) with a co-dosed second therapeutic agent that is a substrate of cytochrome P-450 (CYP), a superfamily of enzymes, that include CYP3A4/A5, CYP2D6, CYP2C19, CYP2C9, and CYP1A2, which metabolize various drugs. This disclosure is based, at least in part, on the discovery that when humans are co-dosed with rivoceranib and one or more substrates of CYP3A4/A5, CYP2D6, CYP2C19, or CYP1A2, the pharmacokinetics of each substrate are altered due to the effect of rivoceranib on the CYP enzyme or enzymes that metabolizes each substrate. In particular, rivoceranib inhibits the metabolism effect of CYP2D6, CYP2C19, and CYP3A4/A5, while having less of an effect on CYP2C9 and CYP1A2.

In one aspect, the present disclosure provides methods of treating cancer, wherein the methods include administering to a patient in need thereof a therapeutically effective amount of rivoceranib while avoiding a concomitant administration of a second therapeutic agent that is a substrate of cytochrome P-450 (CYP) selected from any one or more of CYP3A4/A5, CYP2D6, CYP2C19, or CYP1A2.

In another aspect, the present disclosure provides methods of treating cancer, wherein the methods include determining if a patient in need of a cancer therapy is receiving administration of a substrate of cytochrome P-450 (CYP) selected from any one or more of CYP3A4/A5. CYP2D6, CYP2C19, or CYP1A2; and administering to the patient a therapeutically effective amount of rivoceranib while avoiding a concomitant administration to the patient of the substrate.

In another aspect, the present disclosure provides methods of treating cancer, wherein the methods include discontinuing administration of a substrate of cytochrome P-450 (CYP) selected from any one or more of CYP3A4/A5, CYP2D6, CYP2C19, or CYP1A2 for a time period of about 5 or more half-lives of the substrate; and thereafter administering to the patient a therapeutically effective amount of rivoceranib.

In another aspect, the present disclosure provides methods of treating cancer, wherein the methods include administering to a patient in need thereof a therapeutically effective amount of rivoceranib, wherein the patient is receiving concomitant administration of an adjusted daily dosage of a substrate of cytochrome P-450 (CYP) selected from any one or more of CYP3A4/A5, CYP2D6, or CYP2C19, wherein the adjusted daily dosage of the substrate is about 25% to about 95%, e.g., 30%, 35%, 40%, 45%, 50%, 55%, 60%, 65%, 70%, 75%, 80%, 85%, or 90%, of an intended daily dosage of the substrate, and wherein the intended daily dosage of the substrate is a dosage suitable for the patient if the patient is not receiving concomitant rivoceranib.

In another aspect, the present disclosure provides methods of treating cancer, wherein the methods include administering to a patient in need thereof a therapeutically effective amount of rivoceranib, wherein the patient is receiving concomitant administration of a second therapeutic agent that is a substrate of cytochrome P-450 (CYP) selected from any one or more of CYP3A4/A5, CYP2D6, CYP2C19, or CYP1A2, and any one or more of the following: (a) advising the patient that the substrate should be avoided or discontinued: (b) advising the patient that use of rivoceranib in patients being treated with the substrate is contraindicated; (c) advising the patient that the concomitant administration of rivoceranib and the substrate can alter a therapeutic effect of the substrate; and (d) advising the patient that the substrate should be used with caution in patients receiving rivoceranib due to a potential change in pharmacokinetics of the substrate.

In some embodiments of these different methods, the substrate is dextromethorphan. In some embodiments, the CYP is CYP3A4/A5. In some embodiments, the CYP is CYP3A4. In some embodiments, the CYP is CYP3A5.

In some embodiments, the CYP is CYP2D6. In some embodiments, the CYP is CYP2C19. In some embodiments, the CYP is CYP1A2. In some embodiments, the CYP is CYP2C9.

In another aspect, the present disclosure provides compositions comprising or consisting of rivoceranib or a pharmaceutically acceptable salt thereof for use in the treatment of cancer, in combination with a second therapeutic agent that is a substrate of CYP, for example, any one or more of CYP2D6, CYP2C19, CYP3A4/A5, CYP2C9, and CYP1A2, wherein the dosage of the second therapeutic agent is adjusted according to the treatment regimens recited in the methods disclosed herein, wherein rivoceranib inhibits the metabolism effect of CYP2D6, CYP2C19, and CYP3A4/A5, while having less of an effect on CYP2C9 and CYP1A2.

In various embodiments of the methods and compositions for use as described herein, the cancer is Acanthoma, Acinic cell carcinoma, Acoustic neuroma, Acral lentiginous melanoma, Acrospiroma, Acute eosinophilic leukemia, Acute lymphoblastic leukemia, Acute megakaryoblastic leukemia, Acute monocytic leukemia, Acute myeloblastic leukemia with maturation, Acute myeloid dendritic cell leukemia, Acute myeloid leukemia, Acute promyelocytic leukemia. Adamantinoma, Adenocarcinoma, Adenoid cystic carcinoma, Adenoma, Adenomatoid odontogenic tumor, Adrenocortical carcinoma, Adult T-cell leukemia, Aggressive NK-cell leukemia, AIDS-Related Cancers, AIDS-related lymphoma, Alveolar soft part sarcoma, Ameloblastic fibroma, Anal cancer, Anaplastic large cell lymphoma, Anaplastic thyroid cancer, Angioimmunoblastic T-cell lymphoma, Angiomyolipoma, Angiosarcoma, Appendix cancer, Astrocytoma, Atypical teratoid rhabdoid tumor, Basal cell carcinoma, Basal-like carcinoma, B-cell leukemia, B-cell lymphoma, Bellini duct carcinoma, Biliary tract cancer, Bladder cancer, Blastoma, Bone Cancer. Bone tumor, Brain Stem Glioma, Brain Tumor, Breast Cancer, Brenner tumor, Bronchial Tumor, Bronchioloalveolar carcinoma, Brown tumor, Burkitt's lymphoma, Cancer of Unknown Primary Site, Carcinoid Tumor, Carcinoma, Carcinoma in situ, Carcinoma of the penis, Carcinoma of Unknown Primary Site, Carcinosarcoma, Castleman's Disease. Central Nervous System Embryonal Tumor, Cerebellar Astrocytoma, Cerebral Astrocytoma, Cervical Cancer, Cholangiocarcinoma, Chondroma, Chondrosarcoma, Chordoma, Choriocarcinoma, Choroid plexus papilloma, Chronic Lymphocytic Leukemia, Chronic monocytic leukemia, Chronic myelogenous leukemia, Chronic Myeloproliferative Disorder, Chronic neutrophilic leukemia, Clear-cell tumor, Colon Cancer, Colorectal cancer, Craniopharyngioma, Cutaneous T-cell lymphoma, Degos disease, Dermatofibrosarcoma protuberans, Dermoid cyst, Desmoplastic small round cell tumor, Diffuse large B cell lymphoma, Dysembryoplastic neuroepithelial tumor, Embryonal carcinoma, Endodermal sinus tumor, Endometrial cancer, Endometrial Uterine Cancer, Endometrioid tumor, Enteropathy-associated T-cell lymphoma, Ependymoblastoma, Ependymoma, Epithelioid sarcoma, Erythroleukemia, Esophageal cancer, Esthesioneuroblastoma, Ewing's sarcoma, Extracranial Germ Cell Tumor, Extragonadal Germ Cell Tumor, Extrahepatic Bile Duct Cancer, Extramammary Paget's disease, Fallopian tube cancer, Fetus in fetu, Fibroma, Fibrosarcoma, Follicular lymphoma, Follicular thyroid cancer, Gallbladder Cancer, Ganglioglioma, Ganglioneuroma, Gastric Cancer, Gastric lymphoma. Gastrointestinal cancer, Gastrointestinal Carcinoid Tumor, Gastrointestinal Stromal Tumor, Gastrointestinal stromal tumor, Germ cell tumor, Germinoma, Gestational choriocarcinoma, Gestational Trophoblastic Tumor, Giant cell tumor of bone, Glioblastoma multiforme, Glioma, Gliomatosis cerebri, Glomus tumor, Glucagonoma, Gonadoblastoma, Granulosa cell tumor, Hairy Cell Leukemia, Hairy cell leukemia, Head and Neck Cancer, Heart cancer, Hemangioblastoma, Hemangiopericytoma, Hemangiosarcoma, Hematological malignancy, Hepatocellular carcinoma, Hepatosplenic T-cell lymphoma, Hereditary breast-ovarian cancer syndrome, Hodgkin's lymphoma. Hypopharyngeal Cancer, Hypothalamic Glioma, Inflammatory breast cancer, Intraocular Melanoma, Islet cell carcinoma, Islet Cell Tumor, Juvenile myelomonocytic leukemia, Kaposi Sarcoma, Kaposi's sarcoma, Kidney Cancer, Klatskin tumor, Krukenberg tumor, Laryngeal Cancer, Laryngeal cancer, Lentigo maligna melanoma, Leukemia, Lip and Oral Cavity Cancer, Liposarcoma, Lung cancer, Luteoma, Lymphangioma, Lymphangiosarcoma, Lymphoepithelioma, Lymphoid leukemia, Lymphoma, Macroglobulinemia, Malignant Fibrous Histiocytoma, Malignant Fibrous Histiocytoma of Bone, Malignant Glioma, Malignant Mesothelioma, Malignant peripheral nerve sheath tumor, Malignant rhabdoid tumor, Malignant triton tumor, MALT lymphoma, Mantle cell lymphoma, Mast cell leukemia, Mediastinal germ cell tumor, Mediastinal tumor, Medullary thyroid cancer, Medulloblastoma, Medulloepithelioma, Melanoma, Meningioma, Merkel Cell Carcinoma, Mesothelioma, Mesothelioma, Metastatic Squamous Neck Cancer with Occult Primary, Metastatic urothelial carcinoma, Mixed Mullerian tumor. Monocytic leukemia, Mouth Cancer, Mucinous tumor, Multiple Endocrine Neoplasia Syndrome, Multiple Myeloma, Mycosis fungoides, Myelodysplastic Syndromes, Myeloid leukemia, Myeloid sarcoma, Myeloproliferative Disease, Myxoma, Nasal Cavity Cancer, Nasopharyngeal Cancer, Neoplasm, Neurinoma, Neuroblastoma, Neurofibroma, Neuroma. Nodular melanoma, Non-Hodgkin's Lymphoma, Non-melanoma Skin Cancer, Non-Small Cell Lung Cancer. Ocular oncology, Oligoastrocytoma, Oligodendroglioma. Oncocytoma, Optic nerve sheath meningioma, Oral Cancer, Oral cancer, Oropharyngeal Cancer, Osteosarcoma, Ovarian Cancer, Ovarian Epithelial Cancer, Ovarian Germ Cell Tumor, Ovarian Low Malignant Potential Tumor, Paget's disease of the breast, Pancoast tumor, Pancreatic cancer, Papillary thyroid cancer, Papillomatosis, Paraganglioma, Paranasal Sinus Cancer, Parathyroid Cancer, Penile Cancer. Perivascular epithelioid cell tumor, Pharyngeal Cancer, Pheochromocytoma, Pineal Parenchymal Tumor of Intermediate Differentiation, Pineoblastoma, Pituicytoma, Pituitary adenoma, Pituitary tumor, Plasma Cell Neoplasm, Pleuropulmonary blastoma, Polyembryoma, Precursor T-lymphoblastic lymphoma. Primary central nervous system lymphoma, Primary effusion lymphoma, Primary Hepatocellular Cancer, Primary Liver Cancer, Primary peritoneal cancer, Primitive neuroectodermal tumor, Prostate cancer, Pseudomyxoma peritonei, Rectal Cancer, Renal cell carcinoma, Respiratory Tract Carcinoma Involving the NUT Gene on Chromosome 15, Retinoblastoma, Rhabdomyoma, Rhabdomyosarcoma, Richter's transformation, Sacrococcygeal teratoma, Salivary Gland Cancer, Sarcoma, Schwannomatosis, Sebaceous gland carcinoma, Secondary neoplasm, Seminoma, Serous tumor, Sertoli-Leydig cell tumor, Sex cord-stromal tumor, Sezary Syndrome, Signet ring cell carcinoma, Skin Cancer, Small blue round cell tumor. Small cell carcinoma, Small Cell Lung Cancer, Small cell lymphoma, Small intestine cancer, Soft tissue sarcoma, Somatostatinoma, Soot wart, Spinal Cord Tumor, Spinal tumor, Splenic marginal zone lymphoma, Squamous cell carcinoma, Stomach cancer, Superficial spreading melanoma, Supratentorial Primitive Neuroectodermal Tumor, Surface epithelial-stromal tumor, Synovial sarcoma, T-cell acute lymphoblastic leukemia, T-cell large granular lymphocyte leukemia, T cell leukemia, T-cell lymphoma, T-cell prolymphocytic leukemia, Teratoma, Terminal lymphatic cancer, Testicular cancer, Thecoma, Throat Cancer, Thymic Carcinoma, Thymoma, Thyroid cancer, Transitional Cell Cancer of Renal Pelvis and Ureter, Transitional cell carcinoma, Urachal cancer, Urethral cancer, Urogenital neoplasm, Uterine sarcoma, Uveal melanoma, Vaginal Cancer, Verner Morrison syndrome, Verrucous carcinoma, Visual Pathway Glioma, Vulvar Cancer, Waldenstrom's macroglobulinemia, Warthin's tumor, Wilms' tumor, or any combination thereof.

In some embodiments, the cancer is a liver cancer. In some embodiments, the liver cancer is selected from hepatocellular carcinoma, hepatoma, cholangiocarcinoma, hepatoblastoma, hepatic carcinoma, hepatic angiosarcoma, and metastatic liver cancer. In some embodiments, the cancer is hepatocellular carcinoma.

In some embodiments, the cancer is adenoid cystic carcinoma or hepatocellular carcinoma.

Advantageously, the treatment methods of this disclosure provide a means to manage drug-drug interactions (DDI) caused by the inhibition or induction of certain cytochrome P-450 (CYP) enzymes by rivoceranib and enable the use of additional therapies in combination with rivoceranib to treat cancer. In some embodiments, the treatment methods of this disclosure also provide a means to enhance the pharmacokinetics (i.e., increase AUC) of one or more substrates of CYP3A4/A5, CYP2D6, CYP2C19, and CYP2C9, or reduce the pharmacokinetics (i.e., decrease AUC) of substrates of CYP1A2.

As used herein, the term “subject” can be a vertebrate, such as a mammal, a fish, a bird, a reptile, or an amphibian. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. In one aspect, the subject is a mammal. In one aspect, the subject is a human. In one aspect, the subject is a patient. A patient refers to a subject afflicted with a disease or disorder. The term “patient” includes human and veterinary subjects. In some aspects of the disclosed methods, the subject has been diagnosed with a need for treatment of a disorder of uncontrolled cellular proliferation, such as cancer, e.g., hepatocellular carcinoma, gastric cancer, or adenoid cystic carcinoma.

As used herein, the term “treat,” “treating,” or “treatment” refers to the medical management of a subject with the intent to cure, ameliorate, or stabilize a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In various aspects, the term covers any treatment of a subject, including a mammal (e.g., a human), and includes: (i) inhibiting the disease, i.e., arresting its development; or (ii) relieving the disease, i.e., causing regression of the disease.

A “therapeutically effective amount” when used in connection with a pharmaceutical composition described herein is an amount of one or more pharmaceutically active agent(s) sufficient to produce a therapeutic result in a subject in need thereof.

As used herein, the term “diagnosed or diagnosing” means having been subjected to a physical examination by a person of skill, for example, a physician, and found to have a condition that can be diagnosed or treated by the compounds, compositions, or methods disclosed herein. For example, “diagnosed with a disorder of uncontrolled cellular proliferation” means having been subjected to a physical examination by a person of skill, for example, a physician, and found to have a condition that can be diagnosed or treated by a compound or composition that can inhibit uncontrolled cellular proliferation. Such a diagnosis can be in reference to a disorder, such as a disorder of uncontrolled cellular proliferation, cancer and the like, as discussed herein.

As used herein, the term “assessing” or “assessed” refers to a form of measurement, including determining if a disease or disorder is present or not, as well as, in some instances, determining the amount of something present and/or determining the change of the amount of something present over time. In some embodiments, assessing a subject can lead to a subject being identified to be in need of treatment for a disorder. As used herein, the phrase “identified to be in need of treatment for a disorder,” or the like, refers to selection of a subject based upon need for treatment of the disorder. For example, a subject can be identified as having a need for treatment of a disorder based upon an earlier diagnosis by a person of skill and thereafter subjected to treatment for the disorder. It is contemplated that the identification can, in one aspect, be performed by a person different from the person making the diagnosis. It is also contemplated, in a further aspect, that the administration can be performed by one who subsequently performed the administration.

As used herein, “substrate” is a molecule, e.g., a drug, which binds to the active site of a CYP enzyme to form an enzyme-substrate complex.

As used herein. “AUC” refers to the area under the curve, or the integral, of the plasma concentration of an active pharmaceutical ingredient or metabolite over time following a dosing event.

As used herein “AUC₀₋ₜ” is the integral under the plasma concentration curve from time 0 (dosing) to time “t”.

As used herein, “AUC_{0-∞}” is the AUC from time 0 (dosing) to time infinity. Unless otherwise stated, AUC refers to AUC_{0-∞}. Often a drug is packaged in a salt form, for example rivoceranib mesylate salt, and the dosage form strength refers to the mass of this salt form or the equivalent mass of the corresponding free base form of rivoceranib.

As used herein, “sensitive” substrates of CYP are molecules whose exposure has been shown to be significantly altered by CYP inducers and/or inhibitors in studies. Following the guidelines described in U.S. Department of Health and Human Services Food and Drug Administration Center for Drug Evaluation and Research (CDER) January 2020 Clinical Pharmacology guidance of industry for cytochrome P450 drug interactions, the AUC of a “sensitive” CYP substrate is increased at least 5-fold in the presence of a “strong” CYP inhibitor; a “moderate” CYP inhibitor increases the AUC of a “sensitive” CYP substrate by ≥2- to <5-fold: a “weak” CYP inhibitor increases the AUC of a “sensitive” CYP substrate by ≥1.25- to <2. Conversely, a strong CYP inducer decreases the AUC of a sensitive CYP substrate by ≥80 percent; a moderate CYP inducer decreases the AUC of a sensitive CYP substrate by ≥50 to <80 percent; and a weak CYP inducer decreases the AUC of a sensitive CYP substrate by ≥20 to <50 percent. As described in the foregoing guidance (and demonstrated in the Examples), a DDI study with a representative substrate(s) of CYP isozymes should be conducted for specific CYP substrates. A comparison of AUC of substrates with and without rivoceranib administration will determine whether a substrate is a strong or moderate substrate.

As used herein, Cₘₐₓ is a pharmacokinetic parameter denoting the maximum observed blood plasma concentration following delivery of an active pharmaceutical ingredient. Cₘₐₓ occurs at the time of maximum plasma concentration, tₘₐₓ.

As used herein, “tₘₐₓ” is a pharmacokinetic parameter denoting the time to maximum blood plasma concentration following delivery of an active pharmaceutical ingredient

As used herein, “t_{1/2}” or “plasma half-life” or “elimination half-life” or the like is a pharmacokinetic parameter denoting the apparent plasma terminal phase half-life, i.e., the time, after absorption and distribution of a drug is complete, for the plasma concentration to fall by half.

As used herein, the term “adverse event” (EA or EAs for plural) is any untoward medical occurrence in a patient or clinical investigation subject administered a pharmaceutical product and which does not necessarily have a causal relationship with the treatment. An adverse event (AE) can therefore be any unfavorable and unintended sign (including an abnormal laboratory finding), symptom, or disease temporally associated with the use of a medicinal (investigational) product, whether or not related to the medicinal (investigational) product.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration, intraurethral administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition.

As used herein, the term “pharmaceutically acceptable carrier” relates to pharmaceutically-acceptable, nontoxic carriers or diluents, which are defined as vehicles commonly used to formulate pharmaceutical compositions for animal or human administration. Such carriers may include, however not limited to, buffering agents, solubilizing agents, stabilizing agents or taste additives.

As used herein, “pharmacokinetics” is the movement of drug into, through, and out of the body, i.e., the time-course of its absorption, bioavailability, distribution, metabolism, and excretion.

As used herein. “contraindicated” or “contraindication” is a specific situation in which a drug, procedure, or surgery should not be used because it may be harmful to the person. There are two types of contraindications: (a) relative contraindication means that caution should be used when two drugs or procedures are used together. (It is acceptable to do so if the benefits outweigh the risk.), and (b) absolute contraindication means that event or substance could cause a life-threatening situation. A procedure or medicine that falls under this category must be avoided.

As used herein, the term “intended daily dose” is the amount of a medicine or drug that a patient takes based on a drug label or as instructed by a medical professional in the absence of any DDI.

As used herein, “clinical observation” is the act of measuring, questioning, evaluating, or otherwise observing a patient or a specimen from a patient.

As used herein, “self-report” or “self-reporting” is the act of measuring, evaluating, or otherwise observing the state of the patient from the patient's perspective. Self-reporting can be done through in-person, phone, or virtual interview, questionnaire forms, internet interface, or an application, e.g., phone “app.”

As used herein, “pharmacological efficacy” is related to the maximum effect that a particular drug is capable of producing.

In this application, the use of the singular includes the plural unless specifically stated otherwise. It must be noted that, as used in the specification, the singular forms “a,” “an,” and “the” include plural referents unless the context clearly dictates otherwise. In this application, the use of “or” means “and/or” unless stated otherwise. Furthermore, use of the term “including” as well as other forms, such as “include.” “includes.” and “included,” are not limiting.

As used herein, ranges and amounts can be expressed as “about” a particular value or range. About also includes the exact amount. Hence “about 5 μl” means “about 5 μl” and also “5 μl.” Generally, the term “about” includes an amount that would be expected to be within experimental error. Unless specifically stated or obvious from context, as used herein, the term “about” in reference to a number or range of numbers is understood to mean the stated number and numbers +/−10% thereof, or 10% below the lower listed limit and 10% above the higher listed limit for the values listed for a range.

Unless defined otherwise, all technical and scientific terms used herein have the same meaning as is commonly understood by one of skill in the art to which the claimed subject matter belongs. It is to be understood that the detailed description provides examples and explanations only, which are not restrictive of any subject matter claimed. All publications, patent applications, patents, and other references mentioned herein are incorporated by reference in their entirety. In case of conflict, the present specification, including definitions, will control. In addition, the materials, methods, and examples are illustrative only and not intended to be limiting.

Although methods and materials similar or equivalent to those described herein can be used in the practice or testing of the present invention, suitable methods and materials are described below. Although various features of the disclosure may be described in the context of a single embodiment, the features may also be provided separately or in any suitable combination. Conversely, although the disclosure may be described herein in the context of separate embodiments for clarity, the disclosure may also be implemented in a single embodiment.

Reference in the specification to “some embodiments.” “an embodiment,” “one embodiment,” or “other embodiments” means that a particular feature, structure, or characteristic described in connection with the embodiments is included in at least some embodiments, but not necessarily all embodiments, of the disclosures.

The section headings used herein are for organizational purposes only and are not to be construed as limiting the subject matter described.

Other features and advantages of the methods and compositions of the disclosure will be apparent from the following detailed description, and from the claims.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings.

FIG. 1 is a chronological flow chart of the clinical study schedule and procedures for Example 1.

FIG. 2 is a graph showing a Mean Plasma Concentration-Time Curve over time by Treatment Group (cocktail alone (bottom line) or cocktail+200 mg rivoceranib) for Midazolam.

FIG. 3 is a graph showing a Mean Plasma Concentration-Time Curve over time by Treatment Group (cocktail alone (line ending between 20 and 30 hours) or cocktail+200 mg rivoceranib) for 1-hydroxymidazolam.

FIG. 4 is a graph showing a Mean Plasma Concentration-Time Curve over time by Treatment Group (cocktail alone (bottom line) or cocktail+200 mg rivoceranib) for R-warfarin.

FIG. 5 is a graph showing a Mean Plasma Concentration-Time Curve over time by Treatment Group (cocktail alone (bottom line) or cocktail+200 mg rivoceranib) for S-warfarin.

FIG. 6 is a graph showing a Mean Plasma Concentration-Time Curve over time by Treatment Group (cocktail alone (bottom line) or cocktail+200 mg rivoceranib) for Dextromethorphan.

FIG. 7 is a graph showing a Mean Plasma Concentration-Time Curve over time by Treatment Group (cocktail alone (bottom line after about 3 hours) or cocktail+200 mg rivoceranib) for Dextrorphan.

FIG. 8 is a graph showing a Mean Plasma Concentration-Time Curve over time by Treatment Group (cocktail alone (bottom line after about 3 hours) or cocktail+200 mg rivoceranib) for Omeprazole.

FIG. 9 is a graph showing a Mean Plasma Concentration-Time Curve over time by Treatment Group (cocktail alone (bottom line after about 3-4 hours) or cocktail+200 mg rivoceranib) for 5-hydroxyomeprazole.

FIG. 10 is a graph showing a Mean Plasma Concentration-Time Curve over time by Treatment Group (cocktail alone (top line until about 35 hours) or cocktail+200 mg rivoceranib) for Caffeine.

FIG. 11 is a graph showing a Mean Plasma Concentration-Time Curve over time by Treatment Group (cocktail alone (top line between about 5 hours and about 40 hours) or cocktail+200 mg rivoceranib) for Paraxanthine.

FIG. 12 is a chronology of the clinical study schedule and procedures for Example 2.

FIGS. 13A and 13B are graphs showing a Mean Plasma Concentration-Time Curve over time by Treatment Group (cocktail alone or cocktail+700 mg rivoceranib) for (A) Midazolam and (B) 1-(OH) midazolam.

FIGS. 14A and 14B are graphs showing a Mean Plasma Concentration-Time Curve over time by Treatment Group (cocktail alone or cocktail+700 mg rivoceranib) for (A) R-Warfarin and (B) S-Warfarin.

FIGS. 15A and 15B are graphs showing a Mean Plasma Concentration-Time Curve over time by Treatment Group (cocktail alone or cocktail+700 mg rivoceranib) for (A) Dextromethorphan and (B) Dextrorphan.

FIGS. 16A and 16B are graphs showing a Mean Plasma Concentration-Time Curve over time by Treatment Group (cocktail alone or cocktail+700 mg rivoceranib) for (A) Omeprazole and (B) 5-hydroxyomeprazole.

FIGS. 17A and 17B are graphs showing a Mean Plasma Concentration-Time Curve over time by Treatment Group (cocktail alone or cocktail+700 mg rivoceranib) for (A) Caffeine and (B) Paraxanthine.

### DETAILED DESCRIPTION

Described herein are, inter alia, methods for, and compositions for use in, treating proliferative diseases, in particular, methods for treating cancer. The methods include administering to the patient a therapeutically effective amount of rivoceranib or a pharmaceutically acceptable salt thereof, and managing drug-drug interactions (DDI) with a co-dosed second therapeutic agent that is a substrate of cytochrome P-450 (CYP).

For example, in some embodiments, the methods can reduce tumor size in a subject or inhibit the growth of a tumor, e.g., as determined by a diameter, area, or volume measurement or estimation based on imaging the tumor, e.g., using known imaging methods such a x-ray, computed tomography (CT), magnetic resonance imaging (MRI), positron emission tomography (PET), and ultrasound.

In some embodiments, the methods can suppress tumor growth in a subject with cancer by administering to a patient a therapeutically effective amount of rivoceranib while avoiding a concomitant administration to the patient of an intended daily dose of a substrate of cytochrome P-450 (CYP) selected from any one or more of CYP3A4/A5, CYP2D6, CYP2C19, CYP2C9, or CYP1A2, wherein the intended daily dose of the substrate is a daily dose for the patient if the patient were not receiving concomitant rivoceranib.

Advantageously, the treatment methods described herein enable a healthcare provider to manage DDI caused by the inhibition of a CYP enzyme selected from any one or more of CYP3A4/A5. CYP2D6, CYP2C19, or by the induction of the CYP1A2 enzyme, by rivoceranib, and enable the use of additional therapies in combination with rivoceranib to treat cancer. In some embodiments, the treatment methods of this disclosure also provide a means to enhance the pharmacokinetics (i.e., increase AUC) of one or more substrates of CYP3A4/A5, CYP2D6, CYP2C19, and CYP2C9, or reduce the pharmacokinetics (i.e., decrease AUC) of substrates of CYP1A2. Certain embodiments of administration regimens and cancers treatable by rivoceranib within the present claims, are described herein.

In one aspect, the present disclosure provides methods of treating cancer by administering to a patient in need thereof a therapeutically effective amount of rivoceranib while avoiding a concomitant administration of a second therapeutic agent that is a substrate of cytochrome P-450 (CYP) selected from any one or more of CYP3A4/A5, CYP2D6, CYP2C19, or CYP1A2. For example, the substrate can be only a substrate of CYP3A4/A5, or only of CYP2D6, or only of CYP2C19, or only of CYP1A2, or only of CYP2C9.

In some embodiments, the methods include avoiding administration of pairs of CYP substrates, e.g., substrates of CYP3A4/A5 and CYP2D6, or of CYP3A4/A5 and CYP2C19, or of CYP3A4/A5 and CYP1A2, or of CYP2D6 and CYP2C19, or of CYP2D6 and CYP1A2, or of CYP2C19 and CYP1A2.

In some embodiments, the methods include administering to a patient in need thereof a therapeutically effective amount of rivoceranib while avoiding a concomitant administration of a second therapeutic agent that is a substrate of CYP3A4/A5, CYP2D6, and CYP2C19, or a second therapeutic agent that is a substrate of CYP3A4/A5, CYP2D6, and CYP1A2, or a second therapeutic agent that is a substrate of CYP3A4/A5, CYP2C19, and CYP1A2, or a second therapeutic agent that is a substrate of CYP2D6, CYP2C19, and CYP1A2.

In some embodiments, the methods of treating cancer in a patient in need thereof include (a) discontinuing administration of a substrate of CYP2D6, CYP2C19, and CYP1A2 for a time period of about 5 or more half-lives of the substrate; and thereafter (b) administering to the patient a therapeutically effective amount of rivoceranib.

In these methods, the period for discontinuing administration of a substrate is about 5 to about 6 half-lives of the substrate, about 5 to about 7 half-lives of the substrate. 5 to about 8 half-lives of the substrate, 5 to about 9 half-lives of the substrate, or 5 to about 10 half-lives of the substrate.

In some embodiments, the period for discontinuing administration of a substrate is about 12 to about 24 hours, or about 1 day to about 2 or 3 or 4 or 5 or 6 days. In some embodiments, the period for discontinuing administration of a substrate is about 1 week about 2, 3, or 4 weeks. In some embodiments, the period for discontinuing administration of a substrate is about 1 month to about 2, 3, 4, 5, or 6 months.

In another aspect, the present disclosure provides methods of treating cancer by administering to a patient in need thereof a therapeutically effective amount of rivoceranib, wherein the patient is receiving concomitant administration of an adjusted daily dosage of a substrate of cytochrome P-450 (CYP) selected from any one or more of CYP3A4/A5, CYP2D6, or CYP2C19, wherein the adjusted daily dosage of the substrate is about 25% to about 95% of an intended daily dosage of the substrate, and wherein the intended daily dosage of the substrate is a dosage suitable for the patient if the patient is not receiving concomitant rivoceranib.

In some embodiments of these methods, the adjusted daily dosage of the substrate is about 25% to about 95% of an intended daily dosage of the substrate, or about 35% to about 85% of an intended daily dosage of the substrate, or about 45% to about 75% of an intended daily dosage of the substrate, or about 55% to about 65% of an intended daily dosage of the substrate, or about 25, 30, 35, 40, 45, 50, 55, 60, 65, 70, 75, 80, 85, 90, or 95 percent, of an intended daily dosage of the substrate.

In another aspect, the present disclosure provides methods of treating cancer by administering to a patient in need thereof a therapeutically effective amount of rivoceranib, wherein the patient is receiving concomitant administration of a second therapeutic agent that is a substrate of cytochrome P-450 (CYP) selected from any one or more of CYP3A4/A5, CYP2D6, CYP2C19, or CYP1A2, and any one or more of the following: (a) advising the patient that the substrate should be avoided or discontinued; (b) advising the patient that use of rivoceranib in patients being treated with the substrate is contraindicated: (c) advising the patient that the concomitant administration of rivoceranib and the substrate can alter a therapeutic effect of the substrate; and (d) advising the patient that the substrate should be used with caution in patients receiving rivoceranib due to a potential change in pharmacokinetics of the substrate.

In all of these methods, the substrates or sensitive substrate of cytochrome P-450 (CYP) can be selected from substrates or sensitive substrates of any one or more of CYP3A4/A5, CYP2D6, CYP2C19, or CYP1A2 (e.g., any one individually, or any combination of two, e.g., CYP3A4/A5 and CYP2D6, CYP3A4/A5 and CYP2C19, or CYP3A4/A5 and CYP1A2, or CYP2D6 and CYP2C19, or CYP2C19 and CYP1A2, or CYP2D6 and CYP1A2, or other combinations of two, or any combination of three, e.g., CYP3A4/A5, CYP2D6, and CYP2C19, or CYP3A4/A5, CYP2D6, and CYP1A2, or CYP2D6, CYP2C19, and CYP1A2, or other combinations of three.

In some embodiments of the various methods, the substrate is a substrate or sensitive substrate of CYP3A4/A5. In some embodiments, the substrate is a substrate or sensitive substrate of CYP2D6. In some embodiments, the substrate is a substrate or sensitive substrate of CYP2C19. In some embodiments, the substrate is a substrate or sensitive substrate of CYP1A2. In some embodiments, the substrate is a substrate or sensitive substrate of CYP2C9. These embodiments include any combinations of two, three, or all four of the substrates and/or sensitive substrates.

In some embodiments of the various methods, the adjusted daily dosage of a substrate of CYP selected from any one or more of CYP3A4/A5, CYP2D6, or CYP2C19 is the amount that provides t_{1/2} values substantially the same as t_{1/2} values when the CYP substrate is administered alone. In some embodiments, the targeted t_{1/2} value for a patient who is receiving concomitant administration of rivoceranib and a sensitive substrate of CYP selected from any one or more of CYP3A4/A5, CYP2D6, or CYP2C19 is substantially the same as the t_{1/2} value if the patient is receiving administration of the strong CYP substrate alone.

In some embodiments of the various methods, the targeted t_{1/2} value for a patient who is receiving concomitant administration of rivoceranib and a substrate or sensitive substrate of CYP3A4/A5 is substantially the same as the t_{1/2} value if the patient is receiving administration of the substrate alone. In some embodiments, the targeted t_{1/2} value for a patient who is receiving concomitant administration of rivoceranib and a substrate or sensitive substrate of CYP2D6 is substantially the same as the t_{1/2} value if the patient is receiving administration of the substrate alone. In some embodiments, the targeted t_{1/2} value for a patient who is receiving concomitant administration of rivoceranib and a substrate or sensitive substrate of CYP2C19 is substantially the same as the t_{1/2} value if the patient is receiving administration of the substrate alone.

In some embodiments of these various methods, the adjusted daily dosage of a substrate or sensitive substrate of CYP1A2 is the amount that provides t_{1/2} values or Cₘₐₓ values substantially the same as t_{1/2} values when the CYP substrate is administered alone provided the adjusted daily dosage of the CYP substrate does not exceed the maximum daily dose (MDD) imposed by the regulating agency, e.g., the U.S. Food and Drug Administration (FDA).

In some embodiments of the various methods, the adjusted daily dosage of a substrate or sensitive substrate of CYP selected from any one or more of CYP3A4/A5, CYP2D6, or CYP2C19 is the amount that provides Cₘₐₓ values substantially the same as Cₘₐₓ values when the CYP substrate is administered alone.

In some embodiments of the various methods, the adjusted daily dosage of a CYP substrate or sensitive substrate selected from any one or more of CYP3A4/A5, CYP2D6, or CYP2C19 is the amount that provides AUC_{0-∞} values substantially the same as AUC_{0-∞} values when the CYP substrate is administered alone.

In some embodiments of the various methods, the adjusted daily dosage of a substrate or sensitive substrate of CYP1A2 is the amount that provides AUC_{0-∞} values substantially the same as AUC_{0-∞} values when the CYP substrate is administered alone provided the adjusted daily dosage of the CYP substrate does not exceed the MDD imposed by the regulating agency, e.g., the FDA.

### Cytochrome P-450 Enzymes

Discovered in 1958, the cytochrome P450 (CYP) family of enzymes exhibit a unique and intense absorption band at 450 nm, hence their name. The CYP enzymes are a group of heme-containing enzymes embedded primarily in the lipid bilayer of the endoplasmic reticulum of hepatocytes that takes part in the metabolism of many drugs, steroids, and carcinogens (FASEB Journal 1992, 6(2):745-748) A number of CYP enzymes (or CYPs as a short-hand notation) are expressed in each mammalian species, including humans (Bibi, Z. Nutr Metab (Lond) 2008, 5, 27). In humans, CYPs are best known for their central role in drug metabolism where they are of critical importance to two of the most significant problems in clinical pharmacology: drug-drug interactions (DDI) and inter-individual variability in drug metabolism.

The current system of nomenclature for the various CYPs employs a three-tiered classification based on the conventions of molecular biology: the family (members of the same family display >40% homology in their amino acid sequences), subfamily (55% homology), and individual gene (Nebert D. et al., DNA and cell biology 10.1 (1991): 1-14). The names are indicated by, respectively, an Arabic numeral (family), a capital letter (subfamily) and another Arabic numeral (gene), e.g., CYP1A2. The enzymes that transform drugs in humans belong to CYP families 1 to 4 and more than 30 human CYPs have been identified to date.

The activities of the CYP2C19 and CYP2D6 enzymes are biomedically distributed in the population, allowing classification of individuals as either extensive metabolizers (EM) or poor metabolizers (PM). The concept that most drug oxidations are catalyzed primarily by a small number of CYP enzymes is important in that the approaches to identifying drug-drug interactions are feasible, both in vivo and in vitro.

Drug oxidation by CYPs conforms to the following stoichiometry:

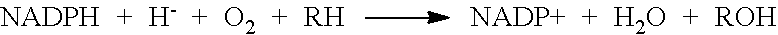

Where RH represents a drug molecule and the co-factor NADPH is Nicotinamide Adenine Dinucleotide Phosphate Hydrogen.

It has been estimated that 90% of drug oxidation can be attributed to six main enzymes. CYP1A2, 2C9, 2C19, 2D6, 2E1, and 3A4/5 (Bertiisson L, et al., Clin Pharmacol Ther 1992, 51(4):388-397). The following CYPs are of interest in the present disclosure.

CYP1A: This family consists of two enzymes, 1A1 and 1A2. CYP1A1 is not significantly expressed in the liver. It is found mainly in the lungs, mammary glands, placenta and lymphocytes. CYP1A2 is expressed mainly in the liver and is induced by cigarette smoking and ingestion of some foodstuffs such as cruciferous vegetables as well as barbecued or charbroiled food. Drugs that are known to be metabolized by CYP1A2 include theophylline, caffeine, imipramine, paracetamol, and phenacitin (Eichelbaum M, Gross, Pharmacology and Therapeutics 1990, 46: 377-394). Alteration in CYP1A2 activity, for example by smoking, may alter the requirements for theophylline among asthmatics and haloperidol among psychiatric patients. Caffeine metabolism is also induced by smoking and explains the increased tolerance to caffeine among smokers (Okuda H, et al., Drug Metabolism and Disposition 1997, 25: 270-273).

CYP2C9: This enzyme plays a significant role in the metabolism, by oxidation, of both xenobiotic and endogenous compounds, and is primarily expressed in liver, duodenum, and small intestine. CYP2CP metabolizes about 100 therapeutic drugs including drugs with a narrow therapeutic index such as warfarin and phenytoin, and other routinely prescribed drugs such as acenocoumarol, tolbutamide, losartan, glipizide, and some nonsteroidal anti-inflammatory drugs.

CYP2C19: This family has a number of commonly used substrates including benzodiazepines (e.g., diazepam, sold under the brand name Valium®), the proton pump inhibitor omeprazole, propranolol, and the anti-depressive amitriptyline. A number of important abnormal variants of this enzyme exist, one of these has important clinical consequences. It has been demonstrated that poor metabolizers who are prescribed proton-pump inhibitor omeprazole as part of therapy against Helicobacter pylori infection may have significantly better clinical outcomes as compared to a group of patients homozygous for the normal, i.e. wild-type, alleles (Pohjola-Sintonen S. et al., European Journal of Clinical Pharmacology 1993, 45: 191-193).

CYP2D6: A large number of drugs are metabolized by this enzyme including a number of anti-arrhythmics such as flecanide and encainide, tricyclic antidepressants, some beta-blockers and a number of selective serotonin re-uptake inhibitors. It is of particular relevance to anesthetics because a number of commonly used analgesics, including codeine and tramadol, are broken down by this enzyme (Mattila M. J., Annals of Medicine 1990, 22: 363-369). To date, more than 70 polymorphisms of CYP2D6 have been catalogued. The majority of these enzymes result in a poor metabolizer phenotype as opposed to the normal, i.e. extensive metabolize phenotype. In addition, a number of genotypes exist where gene duplication results in an ultra-rapid metabolism status. These patients eliminate CYP2D6 substrates faster than normal and in case of pro-drugs such as codeine are at greater risk of opiate related side-effects (Periti P, et al, Clinical Pharmacokinetics 1992, 23: 106-131).

CYP3A4/A5: These liver enzymes metabolize approximately 50% of medications, including many of the statins, benzodiazepines, antibiotics, and antipsychotics. Detection of variants of the CYP3A4 and/or CYP3A5 gene that cause altered enzymatic activity can be used to identify patients who may be at increased risk of having adverse drug reactions while taking standard dosages of 3A4 and/or 3A5 substrates. For example, more than half of the general population (60-80%) possesses inherited differences in 3A5 that cause decreased metabolism. These Decreased Metabolizers may be at increased risk for dose-dependent side effects to drugs normally inactivated by 3A5, and similarly for drugs inactivated by CYP3A4/A5.

### Assessment of Drug-Drug Interactions

In vivo cocktail pathway phenotyping (ICPP) is a relatively new approach that facilitates the simultaneous, but independent, assessment of the activity of multiple metabolic pathways. ICPP is ideally suited for the assessment of the magnitude of in vivo drug-drug interactions (DDIs) involving cytochrome P450 (CYP) enzymes (Snyder, B. D., et al., Eur. J. Clin. Pharmacol. 2014, 70, 1115-1122). Validated drug cocktails containing CYP substrates include the “Cooperstown” (Streetman, D. S et al., Clin. Pharmacol. Ther. 2000, 68, 375-383), “Karolinska” (Christensen, M., et al., Clin. Pharmacol. Ther. 2003, 73, 517-528), and “Inje” (Ryu, J. Y., et al. Clin. Pharmacol. Ther. 2007, 82, 531-540) cocktails. Each cocktail has advantages and disadvantages, and in some cases they have been modified to expand their capacity or address limitations.

The four probe drugs in the Cooperstown cocktail; caffeine (CYP1A2), omeprazole (CYP2C19), dextromethorphan (CYP2D6), and intravenous midazolam (CYP3A), are generally considered the core ‘probes’ of the CYP cocktail approach. These drugs do not interact with each other and are not associated with significant adverse effects. However, the intravenous administration of midazolam (probe for CYP3A) precludes the assessment of gastrointestinal CYP3A enzyme activity. Furthermore, this cocktail does not contain a probe drug for CYP2C9, which accounts for approximately 18% of the CYP protein in the human liver (Miners, J. O., et al, J. Clin. Pharmacol. 1998, 45, 525-538). These limitations are partially ameliorated by using warfarin as a probe for CYP2C9 in the “Cooperstown 5+1” cocktail (Chainuvati, S., et al. 2003, Clin. Pharmacol. Ther. 74, 437-447).

ICPP-based CYP DDI studies typically assess the impact of interactions following multiple (3 to 5 or more) doses of the postulated “perpetrator,” where an interaction perpetrator is a compound that has the potential to alter the metabolic clearance of other drugs that are CYP substrates.

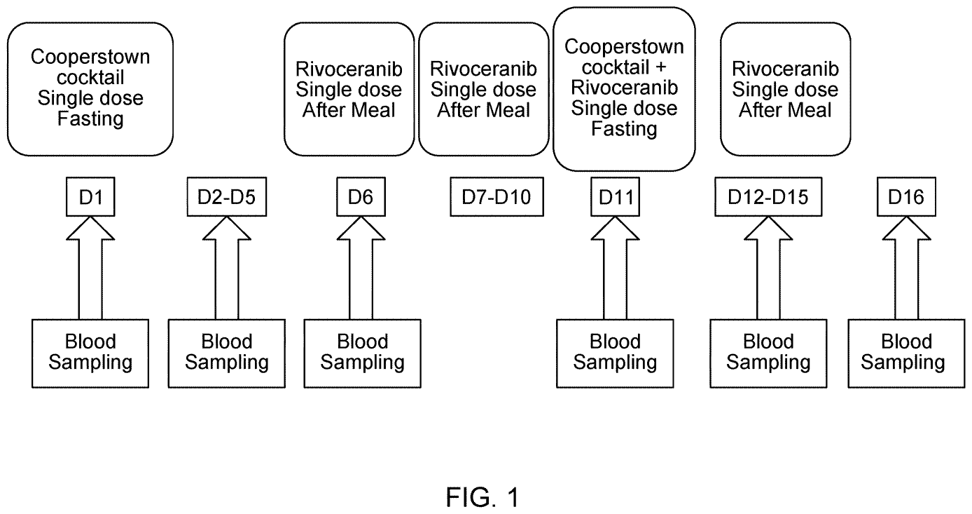

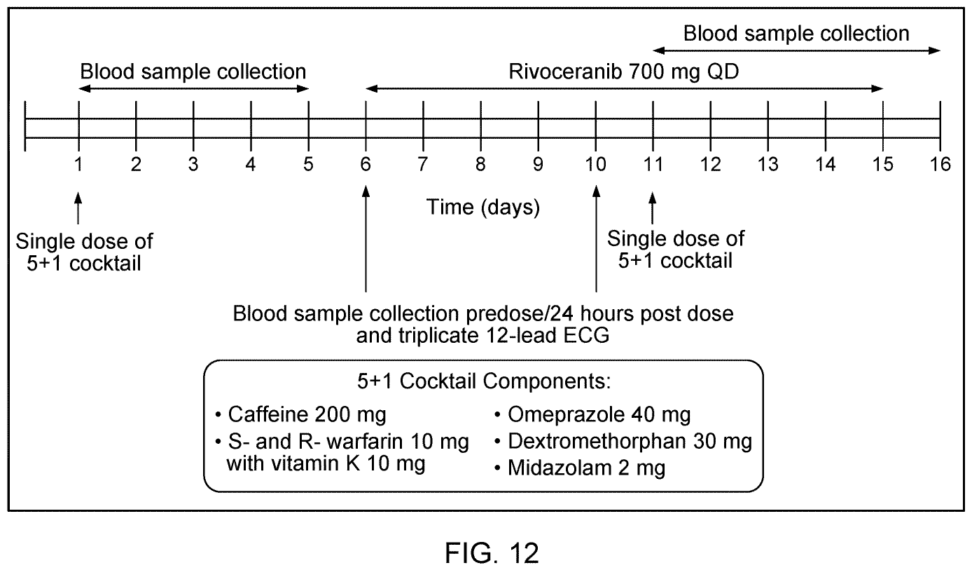

In the present disclosure, in Examples 1 and Example 2, rivoceranib is the “perpetrator” and the validated drug cocktail is Cooperstown 5+1, containing (midazolam [CYP3A4/5 substrate], warfarin [CYP2C9 substrate], dextromethorphan [CYP2D6 substrate], omeprazole [CYP2C19 substrate], and caffeine [CYP1A2 substrate]). According to the clinical design (Example 1, see FIG. 1 and Example 2, see FIG. 12), on the first day (D1) each subject is administered Cooperstown 5+1 Cocktail (midazolam 2 mg, warfarin 10 mg/vitamin K 10 mg, caffeine 200 mg, omeprazole 40 mg, dextromethorphan 30 mg) orally. From D6-D15, each subject is given rivoceranib (200 mg in Example 1 and 700 mg in Example 2) after a meal, with each subject concomitantly administered Cooperstown Cocktail on D11. Blood samples are collected on D1-D6, and D11-D15. A comparison of the exposure (Cₘₐₓ and AUC) of each CYP substrate on D1 with the exposure on D11-D15 then provides a measure of the effect of rivoceranib on the metabolism of each CYP substrate.

### Rivoceranib (Apatinib)

Generally, rivoceranib is an organic heterocyclic chemical compound of having the following chemical formula.

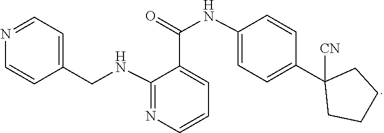 [SMILES: N#CC1(c2ccc(NC(=O)c3cccnc3NCc3ccncc3)cc2)CCCC1]

Rivoceranib (chemical name N-[4-(1-cyanocyclopentyl)phenyl]-2-{[(pyridin-4-yl)methyl]amino}pyridine-3-carboxamide, also known as YN968D1, developed in China as apatinib and marketed as Aitan®) is an orally administered small molecule tyrosine kinase inhibitor. It selectively inhibits vascular endothelial growth factor receptor (VEGFR)-2 leading to blockage of tumor vascular angiogenesis, diminishes survival of existing blood vessels, and retards growth of tumors. Proliferation of endothelial cells is targeted directly, and inhibition of the release of proangiogenic growth factors by cancer or stromal cells is targeted indirectly.

Provided herein are methods of treating cancer in a patient with a therapeutically effective amount of rivoceranib while managing drug-drug interactions (DDI) with a co-dosed second therapeutic agent that is a substrate of cytochrome P-450 (CYP) selected from any one or more of CYP3A4/A5. CYP2D6, CYP2C19, or CYP1A2. The methods include administering rivoceranib or a pharmaceutically acceptable salt thereof to the subject. In some embodiments, rivoceranib or a pharmaceutically acceptable salt thereof is administered in a total daily amount in a range of about 100 mg to about 700 mg. As used herein, the amount of rivoceranib or a pharmaceutically acceptable salt thereof is provided in the mass of the rivoceranib free base form, even though the rivoceranib can be provided as a pharmaceutically acceptable salt thereof.

For example, when the disclosure describes the amount of rivoceranib or a pharmaceutically acceptable salt thereof administered is about 700 mg, the actual drug administered could be 700 mg of rivoceranib free base or the corresponding amount of rivoceranib pharmaceutically acceptable salt thereof, which would be about 869 mg of a rivoceranib mesylate salt. As another example, when the disclosure describes the amount of rivoceranib or a pharmaceutically acceptable salt thereof administered is about 200 mg, the actual drug administered could be 200 mg of rivoceranib free base or the corresponding amount of rivoceranib pharmaceutically acceptable salt thereof, which would be about 248 mg of a rivoceranib mesylate salt. In another example, when the disclosure describes the amount of rivoceranib or a pharmaceutically acceptable salt thereof administered is about 100 mg, the actual drug administered could be 100 mg of rivoceranib free base or the corresponding amount of rivoceranib pharmaceutically acceptable salt thereof, which would be about 124 mg of a rivoceranib mesylate salt.

In some embodiments, the rivoceranib is administered in the form of a free base. In some embodiments, the rivoceranib is administered in the form of a pharmaceutically acceptable salt, such as a mesylate salt form, e.g., having the following chemical structure:

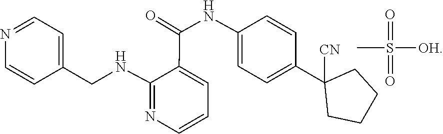

The rivoceranib mesylate has a CAS Registry no. 1218779-75-9, molecular weight of 493.58, and empirical formula C₂₅H₂₇N₅O₄S.

Suitable examples of pharmaceutically acceptable salts include metal salts, such as sodium salts, potassium salts, and lithium salts; alkaline earth metals, such as calcium salts, magnesium salts, and the like; organic amine salts, such as triethylamine salts, pyridine salts, picoline salts, ethanolamine salts, triethanolamine salts, dicyclohexylamine salts, N,N′-dibenzylethylenediamine salts, and the like; inorganic acid salts such as hydrochloride salts, hydrobromide salts, sulfate salts, phosphate salts, and the like; organic acid salts such as formate salts, acetate salts, trifluoroacetate salts, maleate salts, tartrate salts, and the like; sulfonate salts such as methanesulfonate salts, benzenesulfonate salts, p-toluenesulfonate salts, and the like; and amino acid salts, such as arginate salts, asparaginate salts, glutamate salts, and the like. Pharmaceutically acceptable salts also include bitartrate, bitartrate hydrate, bitartrate hemipentahydrate, pentafluoropropionate, mucate, oleate, phosphate dibasic, phosphate monobasic, acetate trihydrate, bis(heptafuorobutyrate), bis(pentafluoropropionate), bis(pyridine carboxylate), bis(trifluoroacetate), chlorhydrate, and sulfate pentahydrate. Other representative pharmaceutically acceptable salts include, e.g., water-soluble and water-insoluble salts, such as the amsonate(4,4-diaminostilbene-2,2-disulfonate), benzonate, bicarbonate, bisulfate, borate, butyrate, calcium edetate, camphorsulfonate, camsylate, carbonate, citrate, clavulariate, dihydrochloride, edetate, edisylate, estolate, esylate, fumarate, fumarate, gluceptate, gluconate, glycollylarsanilate, hexafluorophosphate, hexylresorcinate, hydrabamine, hydroxynaphthoate, iodide, isothionate, lactate, lactobionate, laurate, malate, mandelate, mesylate, methylbromide, methylnitrate, methylsulfate, napsylate, nitrate. N-methylglucamine ammonium salt. 3-hydroxy-2-naphthoate, oxalate, palmitate, pamoate, pantothenate, phosphate/diphosphate, picrate, polygalacturonate, propionate, salicylate, stearate, succinate, sulfosalicylate, suramate, tannate, teoclate, tosylate, triethiodide, and valerate salts.

### Dosing

In some instances, drug dosages are determined as a factor of patient body surface area (BSA). In some instances, BSA is a better indicator of metabolic mass than body weight because it is less affected by abnormal adipose mass, e.g., a patient with a larger BSA would presumably have larger organs for a drug to clear through. Indeed, there can be a 4-10 fold variation in drug clearance between individuals.

Various formulae exist, using height and weight, to calculate BSA without direct measurement. The most widely used is the Du Bois formula, which has been shown to be equally as effective in estimating BSA in obese and non-obese patients.

$$BSA = 0.0 \; 07184 \times {W}^{0.425} \times {H}^{0.725}$$

where W is mass in kg, and H is height in cm. The average adult male BSA is 2.060 m². The average adult female BSA is 1.830 m².

In some instances, drug dosages can be determined based upon the subject's body weight or response to the drug. e.g., adverse events other than disease progression. For example, when a subject is administered a dose of the drug or multiple doses of the drug and exhibits an adverse event other than disease progression, the dosage of the drug may be reduced or the dosage of the drug may be interrupted, e.g., the dosage of the drug is no longer administered daily and can be postponed days without administration or can be administered every other day or less.

In some embodiments, the rivoceranib or a pharmaceutically acceptable salt thereof is administered once daily. In some embodiments, the rivoceranib or a pharmaceutically acceptable salt thereof is administered twice daily. In some embodiments, a total daily amount is administered in a range of about 100 mg to about 700 mg for at least one cycle, wherein each cycle is at least 10 days. In some embodiments, the dosing regimen for rivoceranib or a pharmaceutically acceptable salt thereof in treatment of cancer in a subject comprising rivoceranib or a pharmaceutically acceptable salt thereof, wherein a total daily amount is administered in about 200 mg for at least one cycle. In some embodiments, the dosing regimen for rivoceranib or a pharmaceutically acceptable salt thereof in treatment of cancer in a subject comprising rivoceranib or a pharmaceutically acceptable salt thereof, wherein a total daily amount is administered in about 300 mg for at least one cycle. In some embodiments, the dosing regimen for rivoceranib or a pharmaceutically acceptable salt thereof in treatment of cancer in a subject comprising rivoceranib or a pharmaceutically acceptable salt thereof, wherein a total daily amount is administered in about 500 mg for at least one cycle.

In some embodiments, the cycle is at least 10, 14, 15, 20, 21, 28, 29, 30, or 31 days. In some embodiments, the cycle is at least 28 days, or more. In some embodiments, the rivoceranib or a pharmaceutically acceptable salt thereof is administered in a total daily amount of about 200 mg for at least two cycles, at least three cycles, at least four cycles, at least five cycles, or at least six cycles.

In some embodiments, the rivoceranib or a pharmaceutically acceptable salt thereof is administered, e.g., in a total daily dose, in an amount of at least 100 mg. In some embodiments, the rivoceranib or a pharmaceutically acceptable salt thereof is administered, e.g., in a total daily dose, in an amount of at least 150 mg. In some embodiments, the rivoceranib or a pharmaceutically acceptable salt thereof is administered, e.g., in a total daily dose, in an amount of at least 200 mg. In some embodiments, the rivoceranib or a pharmaceutically acceptable salt thereof is administered, e.g., in a total daily dose, in an amount of at least 225 mg. In some embodiments, the rivoceranib or a pharmaceutically acceptable salt thereof is administered, e.g., in a total daily dose, in an amount of at least 250 mg. In some embodiments, the rivoceranib or a pharmaceutically acceptable salt thereof is administered, e.g., in a total daily dose, in an amount of at least 275 mg.

In some embodiments, the rivoceranib or a pharmaceutically acceptable salt thereof is administered, e.g., in a total daily dose, in an amount of at least 300 mg. In some embodiments, the rivoceranib or a pharmaceutically acceptable salt thereof is administered, e.g., in a total daily dose, in an amount of at least 325 mg. In some embodiments, the rivoceranib or a pharmaceutically acceptable salt thereof is administered, e.g., in a total daily dose, in an amount of at least 350 mg. In some embodiments, the rivoceranib or a pharmaceutically acceptable salt thereof is administered, e.g., in a total daily dose, in an amount of at least 375 mg.

In some embodiments, the rivoceranib or a pharmaceutically acceptable salt thereof is administered, e.g., in a total daily dose, in an amount of at least 400 mg. In some embodiments, the rivoceranib or a pharmaceutically acceptable salt thereof is administered, e.g., in a total daily dose, in an amount of at least 410 mg. In some embodiments, the rivoceranib or a pharmaceutically acceptable salt thereof is administered, e.g., in a total daily dose, in an amount of at least 420 mg. In some embodiments, the rivoceranib or a pharmaceutically acceptable salt thereof is administered, e.g., in a total daily dose, in an amount of at least 425 mg. In some embodiments, the rivoceranib or a pharmaceutically acceptable salt thereof is administered, e.g., in a total daily dose, in an amount of at least 430 mg. In some embodiments, the rivoceranib or a pharmaceutically acceptable salt thereof is administered, e.g., in a total daily dose, in an amount of at least 440 mg. In some embodiments, the rivoceranib or a pharmaceutically acceptable salt thereof is administered, e.g., in a total daily dose, in an amount of at least 450 mg. In some embodiments, the rivoceranib or a pharmaceutically acceptable salt thereof is administered, e.g., in a total daily dose, in an amount of at least 460 mg. In some embodiments, the rivoceranib or a pharmaceutically acceptable salt thereof is administered, e.g., in a total daily dose, in an amount of at least 470 mg. In some embodiments, the rivoceranib or a pharmaceutically acceptable salt thereof is administered, e.g., in a total daily dose, in an amount of at least 475 mg. In some embodiments, the rivoceranib or a pharmaceutically acceptable salt thereof is administered, e.g., in a total daily dose, in an amount of at least 480 mg. In some embodiments, the rivoceranib or a pharmaceutically acceptable salt thereof is administered, e.g., in a total daily dose, in an amount of at least 490 mg.

In some embodiments, the rivoceranib or a pharmaceutically acceptable salt thereof is administered, e.g., in a total daily dose, in an amount of at least 500 mg. In some embodiments, the rivoceranib or a pharmaceutically acceptable salt thereof is administered, e.g., in a total daily dose, in an amount of at least 510 mg. In some embodiments, the rivoceranib or a pharmaceutically acceptable salt thereof is administered, e.g., in a total daily dose, in an amount of at least 520 mg. In some embodiments, the rivoceranib or a pharmaceutically acceptable salt thereof is administered, e.g., in a total daily dose, in an amount of at least 525 mg. In some embodiments, the rivoceranib or a pharmaceutically acceptable salt thereof is administered, e.g., in a total daily dose, in an amount of at least 530 mg. In some embodiments, the rivoceranib or a pharmaceutically acceptable salt thereof is administered, e.g., in a total daily dose, in an amount of at least 540 mg. In some embodiments, the rivoceranib or a pharmaceutically acceptable salt thereof is administered, e.g., in a total daily dose, in an amount of at least 550 mg. In some embodiments, the rivoceranib or a pharmaceutically acceptable salt thereof is administered, e.g., in a total daily dose, in an amount of at least 560 mg. In some embodiments, the rivoceranib or a pharmaceutically acceptable salt thereof is administered, e.g., in a total daily dose, in an amount of at least 570 mg. In some embodiments, the rivoceranib or a pharmaceutically acceptable salt thereof is administered, e.g., in a total daily dose, in an amount of at least 575 mg. In some embodiments, the rivoceranib or a pharmaceutically acceptable salt thereof is administered, e.g., in a total daily dose, in an amount of at least 580 mg. In some embodiments, the rivoceranib or a pharmaceutically acceptable salt thereof is administered, e.g., in a total daily dose, in an amount of at least 590 mg.

In some embodiments, the rivoceranib or a pharmaceutically acceptable salt thereof is administered, e.g., in a total daily dose, in an amount of at least 600 mg. In some embodiments, the rivoceranib or a pharmaceutically acceptable salt thereof is administered, e.g., in a total daily dose, in an amount of at least 610 mg. In some embodiments, the rivoceranib or a pharmaceutically acceptable salt thereof is administered, e.g., in a total daily dose, in an amount of at least 620 mg. In some embodiments, the rivoceranib or a pharmaceutically acceptable salt thereof is administered, e.g., in a total daily dose, in an amount of at least 625 mg. In some embodiments, the rivoceranib or a pharmaceutically acceptable salt thereof is administered, e.g., in a total daily dose, in an amount of at least 630 mg. In some embodiments, the rivoceranib or a pharmaceutically acceptable salt thereof is administered, e.g., in a total daily dose, in an amount of at least 640 mg. In some embodiments, the rivoceranib or a pharmaceutically acceptable salt thereof is administered, e.g., in a total daily dose, in an amount of at least 650 mg. In some embodiments, the rivoceranib or a pharmaceutically acceptable salt thereof is administered, e.g., in a total daily dose, in an amount of at least 660 mg. In some embodiments, the rivoceranib or a pharmaceutically acceptable salt thereof is administered, e.g., in a total daily dose, in an amount of at least 670 mg. In some embodiments, the rivoceranib or a pharmaceutically acceptable salt thereof is administered, e.g., in a total daily dose, in an amount of at least 675 mg. In some embodiments, the rivoceranib or a pharmaceutically acceptable salt thereof is administered, e.g., in a total daily dose, in an amount of at least 680 mg. In some embodiments, the rivoceranib or a pharmaceutically acceptable salt thereof is administered, e.g., in a total daily dose, in an amount of at least 690 mg. In some embodiments, the rivoceranib or a pharmaceutically acceptable salt thereof is administered, e.g., in a total daily dose, in an amount of at least 700 mg.

In some embodiments, the rivoceranib or a pharmaceutically acceptable salt thereof is administered in an amount of from about 100 mg to about 200 mg. In some embodiments, the rivoceranib or a pharmaceutically acceptable salt thereof is administered in an amount of from about 100 mg to about 300 mg. In some embodiments, the rivoceranib or a pharmaceutically acceptable salt thereof is administered in an amount of from about 100 mg to about 400 mg. In some embodiments, the rivoceranib or a pharmaceutically acceptable salt thereof is administered in an amount of from about 100 mg to about 500 mg. In some embodiments, the rivoceranib or a pharmaceutically acceptable salt thereof is administered in an amount of from about 100 mg to about 600 mg.

In some embodiments, the rivoceranib or a pharmaceutically acceptable salt thereof is administered in an amount of from about 100 mg to about 700 mg. In some embodiments, the rivoceranib or a pharmaceutically acceptable salt thereof is administered in an amount of from 100 mg to 750 mg. In some embodiments, the rivoceranib or a pharmaceutically acceptable salt thereof is administered in an amount of from about 100 mg to about 800 mg. In some embodiments, the rivoceranib or a pharmaceutically acceptable salt thereof is administered in an amount of from 100 mg to 850 mg. In some embodiments, the rivoceranib or a pharmaceutically acceptable salt thereof is administered in an amount of from about 100 mg to about 900 mg. In some embodiments, the rivoceranib or a pharmaceutically acceptable salt thereof is administered in an amount of from 150 mg to 850 mg. In some embodiments, the rivoceranib or a pharmaceutically acceptable salt thereof is administered in an amount of from 175 mg to 825 mg. In some embodiments, the rivoceranib or a pharmaceutically acceptable salt thereof is administered in an amount of from 175 mg to 800 mg. In some embodiments, the rivoceranib or a pharmaceutically acceptable salt thereof is administered in an amount of from 175 mg to 700 mg. In some embodiments, the rivoceranib or a pharmaceutically acceptable salt thereof is administered in an amount of from 175 mg to 775 mg. In some embodiments, the rivoceranib or a pharmaceutically acceptable salt thereof is administered in an amount of from 175 mg to 750 mg. In some embodiments, the rivoceranib or a pharmaceutically acceptable salt thereof is administered in an amount of from 175 mg to 700 mg. In some embodiments, the rivoceranib or a pharmaceutically acceptable salt thereof is administered in an amount of from 175 mg to 600 mg. In some embodiments, the rivoceranib or a pharmaceutically acceptable salt thereof is administered in an amount of from 175 mg to 550 mg. In some embodiments, the rivoceranib or a pharmaceutically acceptable salt thereof is administered in an amount of from 175 mg to 500 mg. In some embodiments, the rivoceranib or a pharmaceutically acceptable salt thereof is administered in an amount of from 175 mg to 400 mg. In some embodiments, the rivoceranib or a pharmaceutically acceptable salt thereof is administered in an amount of from 175 mg to 350 mg. In some embodiments, the rivoceranib or a pharmaceutically acceptable salt thereof is administered in an amount of from 175 mg to 300 mg. In some embodiments, the rivoceranib or a pharmaceutically acceptable salt thereof is administered in an amount of from 200 mg to 700 mg. In some embodiments, the rivoceranib or a pharmaceutically acceptable salt thereof is administered in an amount of from 200 mg to 600 mg. In some embodiments, the rivoceranib or a pharmaceutically acceptable salt thereof is administered in an amount of from 200 mg to 550 mg. In some embodiments, the rivoceranib or a pharmaceutically acceptable salt thereof is administered in an amount of from 200 mg to 500 mg. In some embodiments, the rivoceranib or a pharmaceutically acceptable salt thereof is administered in an amount of from 200 mg to 400 mg.

In some embodiments, the rivoceranib or a pharmaceutically acceptable salt thereof is administered in a solid dosage form, such as a tablet. In some embodiments, the tablet comprises the rivoceranib or a pharmaceutically acceptable salt thereof in an amount of about 100 mg. In some embodiments, the tablet comprises the rivoceranib or a pharmaceutically acceptable salt thereof in an amount of about 150 mg. In some embodiments, the tablet comprises the rivoceranib or a pharmaceutically acceptable salt thereof in an amount of about 200 mg. In some embodiments, the tablet comprises the rivoceranib or a pharmaceutically acceptable salt thereof in an amount of about 250 mg. In some embodiments, the tablet comprises the rivoceranib or a pharmaceutically acceptable salt thereof in an amount of about 300 mg. In some embodiments, the tablet comprises the rivoceranib or a pharmaceutically acceptable salt thereof in an amount of about 350 mg.

In some embodiments, the tablet comprises the rivoceranib or a pharmaceutically acceptable salt thereof in an amount of about 400 mg. In some embodiments, the tablet comprises the rivoceranib or a pharmaceutically acceptable salt thereof in an amount of about 450 mg. In some embodiments, the tablet comprises the rivoceranib or a pharmaceutically acceptable salt thereof in an amount of about 500 mg. In some embodiments, the tablet comprises the rivoceranib or a pharmaceutically acceptable salt thereof in an amount of about 550 mg. In some embodiments, the tablet comprises the rivoceranib or a pharmaceutically acceptable salt thereof in an amount of about 600 mg. In some embodiments, the tablet comprises the rivoceranib or a pharmaceutically acceptable salt thereof in an amount of about 650 mg.

In some embodiments, the total daily dose of rivoceranib or a pharmaceutically acceptable salt thereof is administered in one or more 100 mg rivoceranib tablets. In some embodiments, the total daily dose of rivoceranib or a pharmaceutically acceptable salt thereof is administered in two, three, four, five, six, or seven 100 mg rivoceranib tablets. In some embodiments, the rivoceranib or a pharmaceutically acceptable salt thereof is administered in one or more 200 mg rivoceranib tablets. In some embodiments, the total daily dose of rivoceranib or a pharmaceutically acceptable salt thereof is administered in two, three, or four 200 mg rivoceranib tablets. In some embodiments, the rivoceranib or a pharmaceutically acceptable salt thereof is administered in 200 mg rivoceranib tablets and 100 mg rivoceranib tablets. In some embodiments, the total daily dose of rivoceranib or a pharmaceutically acceptable salt thereof is administered in one 200 mg rivoceranib tablet and one 100 mg rivoceranib tablets. In some embodiments, the total daily dose of rivoceranib or a pharmaceutically acceptable salt thereof is administered in one 200 mg rivoceranib tablet and two 100 mg rivoceranib tablets. In some embodiments, the total daily dose of rivoceranib or a pharmaceutically acceptable salt thereof is administered in one 200 mg rivoceranib tablet and three 100 mg rivoceranib tablets.

In some embodiments, the total daily dose of rivoceranib or a pharmaceutically acceptable salt thereof is administered in two 200 mg rivoceranib tablets and one 100 mg rivoceranib tablet. In some embodiments, the total daily dose of rivoceranib or a pharmaceutically acceptable salt thereof is administered in two 200 mg rivoceranib tablets and two 100 mg rivoceranib tablets. In some embodiments, the total daily dose of rivoceranib or a pharmaceutically acceptable salt thereof is administered in two 200 mg rivoceranib tablets and three 100 mg rivoceranib tablets. In some embodiments, the total daily dose of rivoceranib or a pharmaceutically acceptable salt thereof is administered in three 200 mg rivoceranib tablets and one 100 mg rivoceranib tablets.

### Pharmaceutical Compositions

In one aspect, the disclosure relates to pharmaceutical compositions comprising the compounds of the disclosure. That is, a pharmaceutical composition can be provided comprising a therapeutically effective amount of rivoceranib or a pharmaceutically acceptable salt thereof.

In certain aspects, the disclosed pharmaceutical compositions comprise the disclosed compounds (such as rivoceranib or a pharmaceutically acceptable salt thereof) as active ingredients, a pharmaceutically acceptable carrier, and, optionally, other therapeutic ingredients or adjuvants. The pharmaceutical compositions can be conveniently presented in unit dosage form and prepared by any of the methods well known in the art of pharmacy.

In some embodiments, the pharmaceutical compositions containing rivoceranib or a pharmaceutically acceptable salt thereof is administered in an oral liquid, solid, or semisolid dosage form. In some embodiments, the rivoceranib or a pharmaceutically acceptable salt thereof is administered as a solid oral dosage form. In some embodiments, the rivoceranib or a pharmaceutically acceptable salt thereof is administered as a pill, tablet, chewable tablet, specialty tablet, buccal tablet, sublingual tablet, orally-disintegrating tablet, capsule, gel capsule, soft gel capsule, hard gel capsule, sachet, powder, granule, oil-in-water emulsion, water-in-oil liquid emulsion, or crystal or orally dispersible film. In some embodiments, the rivoceranib or a pharmaceutically acceptable salt thereof is administered as a tablet. In some embodiments, the rivoceranib or a pharmaceutically acceptable salt thereof is administered as a solution, suspension, drink, syrup, elixir, ampoule, or dispersion.

In some embodiments, the rivoceranib or a pharmaceutically acceptable salt thereof is administered parenterally. In some embodiments, the rivoceranib or a pharmaceutically acceptable salt thereof is administered intradermaly, subcutaneously, intramuscularly, intraosseously, intraperitoneally or intravenously. In some embodiments, the rivoceranib or a pharmaceutically acceptable salt thereof is administered intraperitoneally. In some embodiments, the rivoceranib or a pharmaceutically acceptable salt thereof is administered intravenously.

When the rivoceranib or a pharmaceutically acceptable salt thereof is administered as a solid oral dosage form, the rivoceranib or a pharmaceutically acceptable salt thereof may be admixed with at least one inert customary excipient (or carrier) such as sodium citrate or dicalcium phosphate, sodium starch, glycolate, colloidal silicon dioxide, or (a) fillers or extenders, for example, starches, lactose, sucrose, glucose, mannitol, and silicic acid, (b) binders, for example, cellulose derivatives, e.g., microcrystalline cellulose, alignates, gelatin, polyvinylpyrrolidone, and gum acacia, (c) humectants, for example, glycerol, (d) disintegrating agents, for example, agar, calcium carbonate, potato or tapioca starch, alginic acid, croscarmellose sodium, complex silicates, and sodium carbonate, (e) solution retarders, for example, paraffin, (f) absorption accelerators, for example, quaternary ammonium compounds, (g) wetting or dispersing agents, for example, cetyl alcohol, glycerol monostearate, magnesium stearate, povidone (K-30), and the like, (h) adsorbents, for example, kaolin and bentonite, and (i) lubricants, for example, talc, calcium stearate, solid polyethylene glycols, sodium lauryl sulfate, or mixtures thereof. In the case of capsules, tablets, and pills, the dosage forms may also comprise buffering agents.

In some embodiments, solid dosage forms may be prepared with coatings and/or shells, such as enteric coatings and others known in the art. For example, they may comprise a film coating system, e.g., which combines polymer, plasticizer, and pigment in a dry concentrate, such as Opadry® white. They may contain pacifying agents, and can also be of such composition that they release the rivoceranib or a pharmaceutically acceptable salt thereof in a certain part of the intestinal tract in a delayed manner. Examples of embedded compositions that can be used are polymeric substances and waxes. The rivoceranib or a pharmaceutically acceptable salt thereof may also be in microencapsulated form, if appropriate, with one or more of the above-mentioned excipients.

In practice, the compounds of the disclosure, or pharmaceutically acceptable salts thereof, of this disclosure can be combined as the active ingredient in intimate admixture with a pharmaceutical carrier according to conventional pharmaceutical compounding techniques. The carrier can take a wide variety of forms depending on the form of preparation desired for administration, e.g., oral or parenteral (including intravenous). In addition to the common dosage forms set out above, the compounds of the disclosure, and/or pharmaceutically acceptable salt(s) thereof, can also be administered by controlled release means and/or delivery devices. The compositions can be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

For example, a tablet containing the composition of this disclosure can be prepared by compression or molding, optionally with one or more accessory ingredients or adjuvants. Compressed tablets can be prepared by compressing, in a suitable machine, the active ingredient in a free-flowing form such as powder or granules, optionally mixed with a binder, lubricant, inert diluent, surface active or dispersing agent. Molded tablets can be made by molding in a suitable machine, a mixture of the powdered compound moistened with an inert liquid diluent.

The pharmaceutical compositions of the present disclosure comprise a compound of the disclosure (or pharmaceutically acceptable salts thereof) as an active ingredient, a pharmaceutically acceptable carrier, and optionally one or more additional therapeutic agents or adjuvants.

Pharmaceutical compositions of the present disclosure suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Pharmaceutical compositions of this disclosure can be in a form suitable for rectal administration wherein the carrier is a solid. It is preferable that the mixture forms unit dose suppositories. Suitable carriers include cocoa butter and other materials commonly used in the art. The suppositories can be conveniently formed by first admixing the composition with the softened or melted carrier(s) followed by chilling and shaping in a mold.

In addition, the pharmaceutical formulations described above can include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions containing a compound of the disclosure, and/or pharmaceutically acceptable salts thereof, can also be prepared in powder or liquid concentrate form.

In another embodiment, the rivoceranib or a pharmaceutically acceptable salt thereof of the present disclosure comprises more than two compositions contained in the same or separate containers, and these at least two compositions may be administered separately, either simultaneously or sequentially. In another embodiment, the rivoceranib or a pharmaceutically acceptable salt thereof of the present disclosure comprises more than two compositions contained in a blister pack, e.g., heat-sealed foil-laminated blister pack or aluminum cold form blister pack.
and these at least two compositions may be administered separately, either simultaneously or sequentially.

### Cancers

Described herein are methods of treating cancer in a subject, where the methods include administering a therapeutically effective amount of rivoceranib while managing drug-drug interactions (DDI) with a co-dosed second therapeutic agent that is a substrate of cytochrome P-450 (CYP) selected from any one or more of CYP3A4/A5, CYP2D6, CYP2C19, CYP2C9, or CYP1A2. In some embodiments, the cancer is improved.

In some embodiments, the cancer is Acanthoma. Acinic cell carcinoma, Acoustic neuroma, Acral lentiginous melanoma, Acrospiroma, Acute eosinophilic leukemia, Acute lymphoblastic leukemia, Acute megakaryoblastic leukemia, Acute monocytic leukemia, Acute myeloblastic leukemia with maturation, Acute myeloid dendritic cell leukemia, Acute myeloid leukemia, Acute promyelocytic leukemia, Adamantinoma, Adenocarcinoma, Adenoid cystic carcinoma, Adenoma, Adenomatoid odontogenic tumor, Adrenocortical carcinoma, Adult T-cell leukemia, Aggressive NK-cell leukemia, AIDS-Related Cancers, AIDS-related lymphoma, Alveolar soft part sarcoma, Ameloblastic fibroma, Anal cancer, Anaplastic large cell lymphoma. Anaplastic thyroid cancer. Angioimmunoblastic T-cell lymphoma, Angiomyolipoma, Angiosarcoma, Appendix cancer, Astrocytoma, Atypical teratoid rhabdoid tumor, Basal cell carcinoma, Basal-like carcinoma, B-cell leukemia, B-cell lymphoma, Bellini duct carcinoma, Biliary tract cancer, Bladder cancer, Blastoma, Bone Cancer, Bone tumor. Brain Stem Glioma, Brain Tumor. Breast Cancer. Brenner tumor, Bronchial Tumor, Bronchioloalveolar carcinoma, Brown tumor, Burkitt's lymphoma, Cancer of Unknown Primary Site, Carcinoid Tumor, Carcinoma, Carcinoma in situ, Carcinoma of the penis, Carcinoma of Unknown Primary Site, Carcinosarcoma, Castleman's Disease. Central Nervous System Embryonal Tumor, Cerebellar Astrocytoma, Cerebral Astrocytoma, Cervical Cancer, Cholangiocarcinoma, Chondroma, Chondrosarcoma, Chordoma, Choriocarcinoma, Choroid plexus papilloma, Chronic Lymphocytic Leukemia, Chronic monocytic leukemia, Chronic myelogenous leukemia, Chronic Myeloproliferative Disorder, Chronic neutrophilic leukemia, Clear-cell tumor, Colon Cancer, Colorectal cancer, Craniopharyngioma, Cutaneous T-cell lymphoma, Degos disease, Dermatofibrosarcoma protuberans, Dermoid cyst, Desmoplastic small round cell tumor, Diffuse large B cell lymphoma, Dysembryoplastic neuroepithelial tumor, Embryonal carcinoma, Endodermal sinus tumor, Endometrial cancer, Endometrial Uterine Cancer, Endometrioid tumor, Enteropathy-associated T-cell lymphoma, Ependymoblastoma, Ependymoma, Epithelioid sarcoma, Erythroleukemia, Esophageal cancer, Esthesioneuroblastoma, Ewing's sarcoma. Extracranial Germ Cell Tumor, Extragonadal Germ Cell Tumor, Extrahepatic Bile Duct Cancer, Extramammary Paget's disease, Fallopian tube cancer, Fetus in fetu, Fibroma, Fibrosarcoma, Follicular lymphoma, Follicular thyroid cancer, Gallbladder Cancer, Ganglioglioma, Ganglioneuroma, Gastric Cancer. Gastric lymphoma, Gastrointestinal cancer, Gastrointestinal Carcinoid Tumor, Gastrointestinal Stromal Tumor, Gastrointestinal stromal tumor, Germ cell tumor, Germinoma, Gestational choriocarcinoma, Gestational Trophoblastic Tumor, Giant cell tumor of bone, Glioblastoma multiforme, Glioma, Gliomatosis cerebri, Glomus tumor, Glucagonoma, Gonadoblastoma, Granulosa cell tumor, Hairy Cell Leukemia, Hairy cell leukemia, Head and Neck Cancer, Heart cancer, Hemangioblastoma, Hemangiopericytoma, Hemangiosarcoma, Hematological malignancy, Hepatocellular carcinoma, Hepatosplenic T-cell lymphoma, Hereditary breast-ovarian cancer syndrome, Hodgkin's lymphoma, Hypopharyngeal Cancer, Hypothalamic Glioma, Inflammatory breast cancer, Intraocular Melanoma, Islet cell carcinoma, Islet Cell Tumor, Juvenile myelomonocytic leukemia, Kaposi Sarcoma, Kaposi's sarcoma, Kidney Cancer, Klatskin tumor, Krukenberg tumor, Laryngeal Cancer, Laryngeal cancer, Lentigo maligna melanoma, Leukemia, Lip and Oral Cavity Cancer, Liposarcoma, Lung cancer, Luteoma, Lymphangioma, Lymphangiosarcoma, Lymphoepithelioma, Lymphoid leukemia, Lymphoma, Macroglobulinemia, Malignant Fibrous Histiocytoma, Malignant Fibrous Histiocytoma of Bone, Malignant Glioma, Malignant Mesothelioma, Malignant peripheral nerve sheath tumor, Malignant rhabdoid tumor. Malignant triton tumor, MALT lymphoma, Mantle cell lymphoma, Mast cell leukemia, Mediastinal germ cell tumor, Mediastinal tumor, Medullary thyroid cancer, Medulloblastoma, Medulloepithelioma, Melanoma, Meningioma. Merkel Cell Carcinoma, Mesothelioma, Mesothelioma, Metastatic Squamous Neck Cancer with Occult Primary, Metastatic urothelial carcinoma, Mixed Mullerian tumor, Monocytic leukemia, Mouth Cancer, Mucinous tumor, Multiple Endocrine Neoplasia Syndrome, Multiple Myeloma, Mycosis fungoides, Myelodysplastic Syndromes, Myeloid leukemia, Myeloid sarcoma, Myeloproliferative Disease, Myxoma, Nasal Cavity Cancer, Nasopharyngeal Cancer, Neoplasm, Neurinoma, Neuroblastoma, Neurofibroma, Neuroma, Nodular melanoma, Non-Hodgkin's Lymphoma, Non-melanoma Skin Cancer, Non-Small Cell Lung Cancer. Ocular oncology, Oligoastrocytoma, Oligodendroglioma. Oncocytoma, Optic nerve sheath meningioma, Oral Cancer, Oral cancer, Oropharyngeal Cancer, Osteosarcoma, Ovarian Cancer, Ovarian Epithelial Cancer, Ovarian Germ Cell Tumor, Ovarian Low Malignant Potential Tumor, Paget's disease of the breast, Pancoast tumor, Pancreatic cancer, Papillary thyroid cancer, Papillomatosis, Paraganglioma. Paranasal Sinus Cancer, Parathyroid Cancer, Penile Cancer, Perivascular epithelioid cell tumor, Pharyngeal Cancer, Pheochromocytoma, Pineal Parenchymal Tumor of Intermediate Differentiation, Pineoblastoma, Pituicytoma, Pituitary adenoma, Pituitary tumor, Plasma Cell Neoplasm, Pleuropulmonary blastoma, Polyembryoma, Precursor T-lymphoblastic lymphoma, Primary central nervous system lymphoma, Primary effusion lymphoma, Primary Hepatocellular Cancer, Primary Liver Cancer, Primary peritoneal cancer, Primitive neuroectodermal tumor, Prostate cancer, Pseudomyxoma peritonei, Rectal Cancer, Renal cell carcinoma. Respiratory Tract Carcinoma Involving the NUT Gene on Chromosome 15. Retinoblastoma, Rhabdomyoma, Rhabdomyosarcoma, Richter's transformation, Sacrococcygeal teratoma, Salivary Gland Cancer, Sarcoma, Schwannomatosis, Sebaceous gland carcinoma, Secondary neoplasm, Seminoma, Serous tumor, Sertoli-Leydig cell tumor, Sex cord-stromal tumor, Sezary Syndrome, Signet ring cell carcinoma, Skin Cancer, Small blue round cell tumor, Small cell carcinoma, Small Cell Lung Cancer, Small cell lymphoma, Small intestine cancer, Soft tissue sarcoma, Somatostatinoma, Soot wart, Spinal Cord Tumor, Spinal tumor, Splenic marginal zone lymphoma, Squamous cell carcinoma. Stomach cancer, Superficial spreading melanoma, Supratentorial Primitive Neuroectodermal Tumor, Surface epithelial-stromal tumor. Synovial sarcoma, T-cell acute lymphoblastic leukemia, T-cell large granular lymphocyte leukemia, T cell leukemia, T-cell lymphoma, T-cell prolymphocytic leukemia, Teratoma, Terminal lymphatic cancer, Testicular cancer, Thecoma, Throat Cancer, Thymic Carcinoma, Thymoma, Thyroid cancer. Transitional Cell Cancer of Renal Pelvis and Ureter, Transitional cell carcinoma, Urachal cancer, Urethral cancer, Urogenital neoplasm, Uterine sarcoma, Uveal melanoma, Vaginal Cancer, Verner Morrison syndrome, Verrucous carcinoma, Visual Pathway Glioma, Vulvar Cancer, Waldenstrom's macroglobulinemia, Warthin's tumor, Wilms' tumor, or any combination thereof.

In some embodiments, the cancer is a liver cancer. In some embodiments, the liver cancer is selected from hepatocellular carcinoma, hepatoma, cholangiocarcinoma, hepatoblastoma, hepatic carcinoma, hepatic angiosarcoma, and metastatic liver cancer. In some embodiments, the cancer is hepatocellular carcinoma.

In some embodiments, the cancer is adenoid cystic carcinoma. In some embodiments, the cancer is recurrent adenoid cystic carcinoma. In some embodiments, the cancer is metastatic adenoid cystic carcinoma. In some embodiments, the adenoid cystic carcinoma is severe adenoid cystic carcinoma.

In some embodiments, the cancer is liver cancer. In some embodiments, the cancer is hepatocellular carcinoma (HCC). In some embodiments, the cancer is early HCC. In some embodiments, the cancer is Intermediate HCC. In some embodiments, the cancer is Advanced HCC. In some embodiments, the cancer is End-Stage HCC.

In some embodiments, the cancer comprises one or more lesions. In some embodiments, the lesion is measured before the treatment and either during the treatment or after the treatment or both. In some embodiments, the lesion is measured by radiological assessments using computerized tomography scan or magnetic resonance imaging. In some embodiments, the lesion has reduced in size after the treatment. In some embodiments, the methods include assessing the cancer, wherein one or more lesions are measured before treatment. In some embodiments, the cancer is hepatocellular carcinoma. In some embodiments, the methods include assessing the hepatocellular carcinoma in a subject, wherein assessing includes measuring one or more lesions in the subject, optionally measuring one or more lesions in the subject over a period of time, e.g., 1 week, 1 month, 2 month, 3 months, 4 months, 5 months, 6 months or more.

In some embodiments, the method of treating cancer includes reducing the size of measurable lesions of a subject. In some embodiments, the lesion has reduced in size by at least 10%. In some embodiments, the lesion has reduced in size by at least 20%. In some embodiments, the lesion has reduced in size by at least 25%. In some embodiments, the lesion has reduced in size by at least 30%. In some embodiments, the lesion has reduced in size by at least 40%. In some embodiments, the lesion has reduced in size by at least 50%. In some embodiments, the lesion has reduced in size by at least 60%. In some embodiments, the lesion has reduced in size by at least 70%. In some embodiments, the lesion has reduced in size by at least 75%. In some embodiments, the lesion has reduced in size by at least 80%. In some embodiments, the lesion has reduced in size by at least 90%. In some embodiments, the cancer has improved. In some embodiments, the hepatocellular carcinoma in a subject has improved. In some embodiments, the methods disclosed herein include discontinuing the administration of the rivoceranib or a pharmaceutically acceptable salt thereof when the subject no longer has cancer.

In some embodiments, the methods disclosed herein are a first line of therapy for treating cancer, e.g., hepatocellular carcinoma. In some embodiments, the methods are a second or a third line of therapy after the prior treatment for the cancer has failed or substantially failed or the disease is substantially refractory to the first line therapy. In some embodiments, a patient has received at least one line of therapy for treating cancer prior to being administered rivoceranib or a pharmaceutically acceptable salt thereof. In some embodiments, the prior line of therapy may be a line of chemotherapy or immunotherapy.

In some embodiments, the methods described herein further comprise administering one or more additional agents selected from the group consisting of anti-cancer agents, anti-proliferative agents, chemotherapeutic agents, immunomodulatory agents, anti-angiogenic agents, anti-inflammatory agents, alkylating agents, steroidal and non-steroidal anti-inflammatory agents, pain relievers, leukotriene antagonists, β2-agonists, anticholinergic agents, hormonal agents, biological agents, immunotherapeutic agents, glucocorticoids, corticosteroid agents, antibacterial agents, antihistamines, anti-malarial agents, anti-viral agents, and antibiotics; and, optionally with radiation therapy.

In some embodiments, the methods described herein further comprise administering one or more additional agents selected from the group consisting of sensitive to moderate CYP3A4/A5 substrates. In some embodiments, the methods described herein further comprise administering one or more additional agents selected from the group consisting of CYP3A4/A5 sensitive/moderate substrates alfentanil, alprazolam, aprepitant, atorvastatin, avanafil, budesonide, buspirone, colchicine, conivaptan, darifenacin, darunavir, dasatinib (anti-cancer), dronedarone, eletriptan, eliglustat, eplerenone, everolimus (anti-cancer), felodipine, ibrutinib (anticancer), indinavir, isavuconazole, ivabradine, lansoprazole, lomitapide, lovastatin, lurasidone, maraviroc, midazolam, mobocertinib, naloxegol, pimozide, quetiapine, rilpivirine, rivaroxaban, saquinavir, sildenafil, simvastatin, sirolimus, tacrolimus, tadalafil, ticagrelor, tipranavir, tolvaptan, triazolam, vardenafil, and venetoclax (anticancer). In some embodiments, the methods described herein further comprise administering one or more additional agents selected from the group consisting of CYP3A4/A5 sensitive/moderate substrates that are anticancer agents, e.g., dasatinib, everolimus, ibrutinib, mobocertinib, and venetoclax.

In some embodiments, the methods described herein further comprise administering one or more additional agents selected from the group consisting of sensitive to moderate CYP2D6 substrates. In some embodiments, the methods described herein further comprise administering one or more additional agents selected from the group consisting of CYP2D6 sensitive/moderate substrates atomoxetine, desipramine, dextromethorphan, eliglustat, imipramine, nebivolol, nisoldipine, nortriptyline, perphenazine, propafenone, propranolol, R-venlafaxine, S-venlafaxine, tolterodine, trimipramine, and tramadol.

In some embodiments, the methods described herein further comprise administering one or more additional agents selected from the group consisting of sensitive to moderate CYP2C19 substrates. In some embodiments, the methods described herein further comprise administering one or more additional agents selected from the group consisting of CYP2C19 sensitive/moderate substrates diazepam, lansoprazole, omeprazole, and rabeprazole.

In some embodiments, the methods described herein further comprise administering one or more additional agents selected from the group consisting of sensitive to moderate CYP2C9 substrates. In some embodiments, the methods described herein further comprise administering one or more additional agents selected from the group consisting of sensitive/moderate CYP2C19 sensitive/moderate substrates including glimepiride, phenytoin, tolbutamide, voriconazole, and warfarin.

In some embodiments, the methods described herein further comprise administering one or more additional agents selected from the group consisting of sensitive to moderate CYP1A2 substrates. In some embodiments, the methods described herein further comprise administering one or more additional agents selected from the group consisting of CYP1A2 sensitive/moderate substrates clozapine, duloxetine, melatonin, pirfenidone, ramelteon, tasimelteon, theophylline, and tizanidine.

In some embodiments, the rivoceranib or a pharmaceutically acceptable salt thereof is administered for 28 days or more. In some embodiments, the rivoceranib or a pharmaceutically acceptable salt thereof is administered daily for at least 1 or 2 months. In some embodiments, the rivoceranib or a pharmaceutically acceptable salt thereof is administered for about 2 months. In some embodiments, the rivoceranib or a pharmaceutically acceptable salt thereof is administered for about 3 months. In some embodiments, the rivoceranib or a pharmaceutically acceptable salt thereof is administered for about 4 months. In some embodiments, the rivoceranib or a pharmaceutically acceptable salt thereof is administered for about 5 months. In some embodiments, the rivoceranib or a pharmaceutically acceptable salt thereof is administered for about 6 months. In some embodiments, the rivoceranib or a pharmaceutically acceptable salt thereof is administered for about 7 months. In some embodiments, the rivoceranib or a pharmaceutically acceptable salt thereof is administered for about 8 months. In some embodiments, the rivoceranib or a pharmaceutically acceptable salt thereof is administered for about 9 months. In some embodiments, the rivoceranib or a pharmaceutically acceptable salt thereof is administered for about 10 months. In some embodiments, the rivoceranib or a pharmaceutically acceptable salt thereof is administered for about 11 months. In some embodiments, the rivoceranib or a pharmaceutically acceptable salt thereof is administered for about 12 months. In some embodiments, the rivoceranib or a pharmaceutically acceptable salt thereof is administered for 2 months or more. It is understood that cancer refers to or describes the physiological condition in mammals that is typically characterized by unregulated cell growth. The cancer may be multi-drug resistant (MDR) or drug-sensitive.

### Methods of Treatment

Some embodiments described herein provide methods of treating cancer including administering to the patient a therapeutically effective amount of rivoceranib in combination with a second small molecule therapy, which is either dosed in tandem or concomitant with rivoceranib in overlapping dosage regimens. In some embodiments, the second small molecule is chosen from a wide variety of compounds with the same or different modes of action directed to the treatment of cancer to complement the pharmacological efficacy of rivoceranib in a synergistic or additive manner. In some embodiments, the second active agent is a small molecule directed to the treatment of indications other than treating cancer, e.g., a small molecule such as warfarin or omeprazole.

In some embodiments, guidance for whether the second small molecule is a substrate of CYP3A4/A5, CYP2D6, CYP2C19, CYP2C9, or CYPTA2 is obtained from the drug Manufacturer's package insert or other sources, see Flockhart DA. Drug Interactions: Cytochrome P450 Drug Interaction Table. Indiana University School of Medicine (2007); fda.gov/drugs/drug-interactions-labeling/drug-development-and-drug-interactions-table-substrates-inhibitors-and-inducers; and straighthealthcare.com/index.html)

As of 2021, there are about 90 anti-cancer small molecules approved in the United States and China (Zhong L, et al., Signal Trans. Target Ther., 2021, May 31; 6(1):201).

In some embodiments, the second therapeutic agent is a small molecule anti-cancer therapeutic agent selected from approved tyrosine kinase inhibitors, including, but not limited to, any one of Crizotinib. Ceritinib, Alectinib, Brigatinib, Lorlatinib, Capmatinib, Tepotinib, Gefitinib, Erlotinib, Lapatinib, Icotinib, Afatinib, Osimertinib, Neratinib, Dacomitinib, Almonertinib, Tucatinib, Midostaurin, Gilteritinib, Quizartinib, Pexidartinib, Sorafenib, Sunitinib, Pazopanib, Vandetanib, Axitinib, Cabozantinib, Regorafenib, Lenvatinib, Tivozanib, Fruquintinib, Nintedanib, Anlotinib, Erdafitinib, Pemigatinib, Avapritinib, Ripretinib. Selpercatinib, Pralsetinib, Larotrectinib, and Entrectinib. Some of these small molecules are non-receptor tyrosine kinase inhibitors including Imatinib, Dasatinib, Nilotinib, Bosutinib, Radotinib, Ponatinib, Ibrutinib, Acalabrutinib. Zanubrutinib, Ruxolitinib, and Fedratinib.

In some embodiments, the small molecule anti-cancer therapeutic is selected from approved Serine/Threonine kinase inhibitors used in cancer therapy including but not limited to any one of Vemurafenib, Dabrafenib, Encorafenib, Trametinib, Cobimetinib, Binimetinib, Selumetinib, Palbociclib, Ribociclib, Abemaciclib, Copanlisib, Duvelisib, Alpelisib. Temsirolimus, Everolimus, and Sirolimus.

In some embodiments, the small molecule anti-cancer therapeutic is selected from approved epigenetic inhibitors used in cancer therapy including but not limited to any one of Tazemetostat, Vorinostat, Romidepsin, Belinostat, Tucidinostat, Panobinostat, Ivosidenib, and Enasidenib.

In some embodiments, the small molecule anti-cancer therapeutic is selected from approved BCL-2, hedgehog pathway, proteasome, and Poly (ADP-ribose) polymerase (PARP) inhibitors including but not limited to any one of Venetoclax, Vismodegib, Sonidegib, Glasdegib, Bortezomib, Carfilzomib, Ixazomib, Olaparib, Rucaparib, Niraparib, and Talazoparib.

Additional small molecule anti-cancer therapeutics currently in clinical trials or in future discovery will add to the preceding lists.

In some embodiments, the small molecule is directed at a wide variety of indications, e.g., different types of cancers.

In some embodiments, the second therapeutic agent is a small molecule that is a sensitive substrate of the CYP3A subfamily, including, but not limited to, any one of Albendazole (Albenza®), Alfentanil (Alfenta®), Aliskiren (Tekturna®), Almotriptan (Axert®), Alprazolam (Xanax®), Amlodipine (Norvasc®), Apremilast (Otezla®), Aprepitant (Emend®), Aripiprazole (Abilify®), Atogepant (Qulipta®), Avanafil (Stendra®), Benzhydrocodone (Apadaz®), Brexpiprazole (Rexulti®), Bromocriptine (Cycloset®, Parlodel®), Budesonide (Entocort®, Pulmicort®), Buprenorphine (Suboxone®, Butrans®, etc.), Butorphanol (Stadol®), Buspirone (Buspar®), Cannabidiol (Epidiolex®), Cariprazine (Vraylar®), Cilostazol (Pletal®), Clindamycin (Cleocin®), Clonazepam (Klonopin®), Codeine, Colchicine (Colcrys®, Mitigare®), Conivaptan (Vaprisol®), Cyclophosphamide (Cytoxan®), Cyclosporine (Neoral®), Gengraf®, Sandimmune®), Dabrafenib (Tafinlar®), Daclatasvir (Daklinza™), Darifenacin (Enablex®), Darunavir (Prezista®), Dasatinib (Sprycel®), Dexlansoprazole (Dexilant®), Diazepam (Valium®), Dienogest (progesterone in oral contraceptives), Dihydrocodeine (Trezix®), Dihydroergotamine (DHE-45®, Migranal®), Disopyramide (Norpace®), Dronedarone (Multaq®), Drospirenone (progesterone in oral contraceptives), Elbasvir (Zepatier®), Eletriptan (Relpax®), Enzalutamide (Xtandi®), Eplerenone (Inspra®), Ergotamine (Cafergot®), Esomeprazole (Nexium®), Eszopiclone (Lunesta®), Ethinyl Estradiol (oral contraceptives, Ortho Evra®, NuvaRing®), Etonogestrel (Implanon®, Nexplanon®, NuvaRing®), Everolimus (Afinitor®), Exemestane (Aromasin®), Felodipine (Plendil®), Fentanyl (Duragesic®), Finerenone (Kerendia®), Flibanserin (Addyi®), Fluticasone (Flovent®, Amuity Ellipta®), Grazoprevir (Zepatier®), Guanfacine (Intuniv®), Hydrocodone (Norco®, Vicodin®), Ibrexafungerp (Brexafemme®), Iloperidone (Fanapt®), Ibrutinib (Imbruvica®), Indinavir (Crixivan®), Ivabradine (Corlanor®), Ivacaftor (Kalydeco®), Lapatinib (Tykerb®), Lansoprazole (Prevacid®), Lefamulin (Xenleta™)Lemborexant (Dayvigo®), Levomilnacipran (Fetzima®), Lomitapi, de (Juxtapid®), Lopinavir and Ritonavir (Kaletra®), Lovastatin (Mevacor®), Lumateperone (Caplyta®), Lurasidone (Latuda®), Maraviroc (Selzentry®), Medroxyprogesterone acetate (Depo-Provera®), Meperidine (Demerol®), Methadone, Midazolam (Versed®), Mirtazapine (Remeron®), Naloxegol (Movanti k®), Nevirapine (Viramune®), Nisoldipine (Sular®)Ospemifene (Osphena®), Oxycodone (Percocet®, Oxycontin®), Paritaprevir (Viekira Pak™, Technivie™), Pimozide (Orap®), Praziquantel (Biltricide®), Propafenone (Rythmol®), Quetiapine (Seroquel®), Quinidine, Repaglinide (Prandin®), Rimegepant (Nurtec®), Risperidone (Risperdal®), Roflumilast (Daliresp®), Salmeterol (Serevent®), Samidorphan (Lybalvi®), Saquinavir (Invirase®), Saxagliptin (Onglyza®), Sildenafil (Viagra®), Simeprevir (Olysio®), Simvastatin (Zocor®), Sirolimus (Rapamune®), Suvorexant (Belsomra®), Tacrolimus (Prograf®), Tadalafil (Cialis®), Tamsulosin (Flomax®), Tasimelteon (Hetlioz®), Temsirolimus (Torisel®), Ticagrelor (Brilinta®), Tinidazole (Tindamax®), Tipranavir (Aptivus®), Tofacitinib (Xeljanz®), Tolvaptan (Samsca®), Tramadol (Ultram®), Triazolam (Halcion®), Upadacitinib (Rinvoq®), Vardenafil (Levitra®), Velpatasvir (Epclusa®), Verapamil (Calan®, Covera-HS®, Verelan®), Vilanterol (Anoro Ellipta®), Vilazodone (Viibryd®), Vorapaxar (Zontivity®), Zolpidem (Ambien®), and Zonisamide (Zonegran™). In some embodiments, medications or foods that are strong inducers or inhibitors of the CYP3A subfamily are to be avoided when rivoceranib is administered.

In some embodiments, the small molecule is a sensitive substrate of the CYP3A4/A5 subfamily, including, but not limited to, any one of Abrocitinib (Cibinqo®), Alfuzosin (Uroxatral®), Amiodarone (Cordarone®), Apixaban (Eliquis®), Atorvastatin (Lipitor®), Beclomethasone (Qvar®), Boceprevir (Victrelis®), Canagliflozin (Invokana™), Carbamazepine (Tegretol®), Cenobamate (Xcopri™), Ciclesonide (Alvesco®, Omnaris®), Clarithromycin (Biaxin®), Clomipramine (Anafranil®), Clopidogrel (Plavix®), Clozapine (Clozaril®), Delavirdine (Rescriptor®), Desvenlafaxine (Pristiq®), Dexamethason (DexPak®, Decadron®), Dextromethorphan (multiple cough suppressants). Diltiazem (Cardizem®, Cartia®), Dolutegravir (Tivicay®), Doxazosin (Cardura, Cardura XL®), Edoxaban (Savaysa®), Enalapril (Vasotec®), Erythromycin (E.E.S.®, Ery-Tab®), Estradiol (Estrace®, Climara®), Etonogestrel (Implanon®, Nexplanon® NuvaRing®), Felodipine (Plendil®), Fluvastatin (Lescol®), Flunisolide (Aerospan®), Glyburide (DiaBeta®), Haloperidol (Haldol®), Hydrocortisone (Kenalog®), Imipramine (Tofranil®), Indacaterol (Arcapta®), Indapamide, Isosorbide dinitrate (Isordil®), Isosorbide mononitrate (ISMO®), Isotretinoin (Accutane®, Claravis®), Isradipine (Dynacirc®), Ivermectin (Stromectol®), Lacosamide (Vimpat®), Letermovir (Prevymis®), Linagliptin (Tradjenta®), Losartan (Cozaar®), Metaxalone (Skelaxin®), Mometasone (Asmanex®), Montelukast (Singulair®), Nateglinide (Starlix®), Nicardipine (Cardene®), Nifedipine (Adalat®, Procardia®), Ondansetron (Zofran®), Ozanimod (Zeposia®), Paliperidone (Invega®), Pantoprazole (Protonix®), Pioglitazone (Actos®), Pitolisant (Wakix®), Prasugrel (Effient®), Progesterone (Provera®), Rabeprazole (Aciphex®), Ramelteon (Rozerem®), Rifabutin (Mycobutin®), Rivaroxaban (Xarelto®), Silodosin (Rapaflo®), Sertraline (Zoloft®), Sitagliptin (Januvia®), Spironolactone (Aldactone®), Tamoxifen, Telaprevir (Incivek®), Telithromycin (Ketek®), Terbinafine (Lamisil®), Testosterone (Androgel®), Tiotropium (Spiriva®), Trazodone. Voriconazole (Vfend®), Voxilaprevir (Vosevi™), Zaleplon (Sonata®), Zileuton (Zyflo®), and Ziprasidone (Geodon®).

In some embodiments, medications or foods that are strong inducers or inhibitors of CYP3A4/A5 are to be avoided when rivoceranib is administered.

In some embodiments, the small molecule is a sensitive substrate of CYP2D6, including, but not limited, to any one of Amphetamines (Adderall®, Vyvanse®), Aripiprazole (Abilify®), Atomoxetine (Strattera®), Brexpiprazole (Rexulti®), Codeine Desipramine (Norpramin®), Dextromethorphan (Robitussin DM®), Dihydrocodeine (Trezix®, Iloperidone (Fanapt®), Methadone, Metoprolol (Toprol®), Nebivolol (Bystolic®), Perphenazine, Pitolisant (Wakix®), Propafenone (Rythmol®), Risperidone (Risperdal®), Timolol (Timoptic®, Blocadren®), Tolterodine (Detrol®), Tramadol (Ultram®), Venlafaxine (Effexor®), Viloxazine (Qelbree®), Vortioxetine (Trintellix®), and Thioridazine (Mellaril®).

In some embodiments, the small molecule is a substrate of CYP2D6, including but not limited to any one of Amitriptyline (Elavil®), Arformoterol (Brovana®), Benzhydrocodone (Apadaz®), Cariprazine (Vravlar®), Carvedilol (Coreg®), Chlorpheniramine (Chlor-Trimeton®), Chlorpromazine (Thorazine®), Ciclesonide (Alvesco®, Omnaris®), Clomipramine (Anafranil®), Clonidine (Catapres®), Clozapine (Clozaril®), Donepezil (Aricept®), Doxazosin (Cardura®, Cardura XL®, Cardura XL® PI). Doxepin, Duloxetine (Cymbalta®), Flecainide (Tambocor®), Fluoxetine (Prozac®), Fluphenazine, Fluvoxamine (Luvox®), Formoterol (Foradil®), Haloperidol (Haldol®), Ibrutinib (Imbruvica®), Imipramine (Tofranil®), Ivermectin (Stromectol®), Letermovir (Prevymis®), Metaxalone (Skelaxin®), Metoclopramide (Reglan®), Mirtazapine (Remeron®), Mexiletine, Nicardipine (Cardene®), Nortriptyline (Pamelor®), Olanzapine (Zyprexa®), Ondansetron (Zofran®), Oxycodone (Oxycontin®, Percocet®), Paliperidone (Invega®), Paroxetine (Paxil®), Promethazine (Phenergan®), Propranolol (Inderal®), Tamoxifen, Tamsulosin (Flomax®), Tiotropium (Spiriva®), Trimipramine (Surmontil®), and Umeclidinium (Anoro Ellipta®).

In some embodiments, medications or foods that are strong inducers or inhibitors of CYP2D6 are to be avoided when rivoceranib is administered.

In some embodiments, the small molecule is a sensitive substrate of CYP2C19, including, but not limited to, any one of Abrocitinib (Cibingo®), Cannabidiol (Epidiolex®), Carisoprodol (Soma®), Cilostazol (Pletal®), Citalopram (Celexa®), Clobazam (Onfi®), Clopidogrel (Plavix®) Dexlansoprazole (Dexilant®), Diazepam (Valium©), Esomeprazole (Nexium®), Flibanserin (Addyi®), Lansoprazole (Prevacid®), Methadone, Omeprazole (Prilosec®), Phenytoin (Dilantin®), and Tofacitinib (Xeljanz®).

In some embodiments, the small molecule is a substrate of CYP2C19, including, but not limited to, any one of Amitriptyline (Elavil®), Apixaban (Eliquis®), Arformoterol (Brovana®), Brivaracetam (Briviact®), Cenobamate (Xcopri™) Chloramphenicol, Clomipramine (Anafranil®), Cyclophosphamide, Doxazosin (Cardura®, Cardura XL®) [4. Cardura XL® PI), Fluoxetine (Prozac®), Formoterol (Foradil®), Imipramine (Tofranil®), Indomethacin (Indocin®), Lacosamide (Vimpat®), Nelfinavir (Viracept®), Nilutamide (Nilandron®), Nortriptyline (Pamelor®) Ospemifene (Osphena®), Pantoprazole (Protonix®), Prasugrel (Effient®), Praziquantel (Biltricide®), Primidone (Mysoline®), Progesterone, Proguanil (Malarone®), Propranolol (Inderal®), Rabeprazole (Aciphex®), Samidorphan (Lybalvi®), Sertraline (Zoloft®), Suvorexant (Belsomra®), Teniposide, Terbinafine (Lamisil®), Timolol (Timoptic®, Blocadren®), Voriconazole (Vfend®), and Warfarin (Coumadin®).

In some embodiments, medications or foods that are strong inducers or inhibitors of CYP2C19 are to be avoided when rivoceranib is administered.

In some embodiments, the small molecule is a sensitive substrate of the CYP1A2 subfamily, including, but not limited to, any one of Alosetron (Lotronex®), Caffeine, Clozapine (Clozaril®), Duloxetine (Cymbalta®), Melatonin, Olanzapine (Zyprexa®), Pirfenidone (Esbriet®), Ramelteon (Rozerem©), Roflumilast (Daliresp®), Ropinirole (Requip®), Tasimelteon (Hetlioz®), Theophylline (Theo-Dur®, Theo-24®), Thioridazine (Mellaril®), and Tizanidine (Zanaflex®).

In some embodiments, the small molecule is a substrate of the CYP1A2 subfamily, including, but not limited to, any one of Amitriptyline (Elavil®), Apixaban (Eliquis®), Apremilast (Otezla®), Asenapine (Saphris®), Clomipramine (Anafranil®), Clopidogrel (Plavix®), Cyclobenzaprine (Flexeril®), Dacarbazine, Estradiol (Estrace®), Febuxostat (Uloric®), Ethanol, Flutamide, Fluvoxamine (Luvox®), Frovatriptan (Frova®), Haloperidol (Haldol®), Imipramine (Tofranil®), Leflunomide (Arava®), Metaxalone (Skelaxin®), Mexiletine, Mirtazapine (Remeron®), Nabumetone (Relafen®), Naproxen (Aleve®), Ondansetron (Zofran®), Pentoxifylline (Trental®), Praziquantel (Biltricide®), Propafenone (Rythmol®), Propranolol (Inderal®), Terbinafine (Lamisil®), Triamterene, Verapamil (Calan®), Voxilaprevir (Vosevi™), Warfarin (Coumadin®), Zileuton (Zyflo®), Zolmitriptan (Zomig®), and Zolpidem (Ambien®)

In some embodiments, medications or foods that are strong inducers or inhibitors of CYP1A2 are to be avoided when rivoceranib is administered.

Some embodiments described herein provide methods of treating cancer including administering to the patient a therapeutically effective amount of rivoceranib in combination with a second small molecule therapy, which is dosed in tandem such that the patient is not exposed to the second small molecule therapy in the presence of rivoceranib. It is understood by one of ordinary skill in the art that such a treatment regimen can, for example, involve two treatment phases, e.g., with a wash-out period in between, to allow exposure levels in the patient to drop to acceptable levels before introducing the next treatment phase.

Some embodiments provide methods of treating cancer including administering to a patient in need thereof a therapeutically effective amount of rivoceranib while avoiding a concomitant administration to the patient of an intended daily dose of a substrate of CYP selected from any one or more of CYP3A4/A5, CYP2D6, CYP2C19, or CYP1A2, wherein the intended daily dose of the substrate is a daily dose for the patient if the patient were not receiving concomitant rivoceranib.

### Recommendations Against the Administration of Certain Combination Therapies

In some embodiments, the second small molecule therapy is a sensitive substrate of CYP1A2 as described herein. Because rivoceranib may cause a reduction in the exposure of the second small molecule therapy due to induction of CYP1A2, concomitant administration of the second small molecule therapeutic that is a sensitive substrate of CYP1A2 is not recommended, particularly if the daily adjusted dosage of the second small molecule therapeutic required to compensate for the rivoceranib effect would exceed a maximum approved dosage.

In some embodiments, the second small molecule therapy is a sensitive substrate of any one or more of CYP3A4/A5, CYP2D6, and CYP2C19, as described herein. Because rivoceranib may cause an increase in the exposure of the second small molecule therapeutic due to inhibition of any one or more of CYP3A4/A5, CYP2D6, and CYP2C19, concomitant administration of the second small molecule therapeutic that is a sensitive substrate of any one or more of CYP3A4/A5, CYP2D6, and CYP2C19 is not recommended. There are several reasons to avoid concomitant administration of the second small molecule therapeutic that is a sensitive substrate of any one or more of CYP3A4/A5, CYP2D6, and CYP2C19.

Without being bound by theory, these reasons include: (i) the second small molecule therapeutic may have a narrow therapeutic window, such that any elevation in exposure may cause an adverse event (AE), (ii) the second small molecule therapeutic may be used to maintain the physical or mental health of a subject, and any change in the exposure levels may influence the intended pharmacology, and (iii) a reduction in dosage of the second small molecule therapeutic, e.g., using a pill cutter or other means, may not be feasible, or the available dosage forms of the second small molecule therapeutic may not be available.

The methods and compositions disclosed herein are further described in the following examples, which do not limit the scope of the claims.

### Example 1

### A Pharmacokinetic Interaction Study Between Rivoceranib and Cytochrome P450 Enzyme (CYP1A2, CYP2C9, CYP2C19, CYP2D6, and CYP3A) Substrates

This example describes a pharmacokinetic interaction study in healthy human volunteers dosed with 200 mg rivoceranib and a mixture of cytochrome P450 enzyme (CYP1A2, CYP2C9, CYP2C19, CYP2D6, and CYP3A) substrates.

### Objectives

The primary objective of the study was to determine the impact of multiple oral doses of rivoceranib on the single oral dose pharmacokinetics (PK) of the CYP substrates included in the Cooperstown 5+1 cocktail (midazolam [CYP3A4/5 substrate], warfarin [CYP2C9 substrate], dextromethorphan [CYP2D6 substrate], omeprazole [CYP2C19 substrate], and caffeine [CYP1A2 substrate]) in healthy subjects. The “+l” in the Cooperstown cocktail refers to vitamin K, which is given together with the warfarin to prevent any anticoagulant effect.

### Methodology

This was an open-label, fixed-sequence, crossover drug interaction study. Subjects underwent screening examinations prior to drug administration. Eligible subjects were admitted to the clinical research unit (CRU) on Day minus two (D−2) for COVID-19 screening (RAPID test). On Day minus one (D−1) subjects were fasted for 10 hours overnight with free access to water. On Day one (D1), each subject was administered Cooperstown 5+1 Cocktail (also referred to as “Cooperstown Cocktail” throughout Example 1) comprised of midazolam 2 mg, warfarin 10 mg/vitamin K 10 mg, caffeine 200 mg, omeprazole 40 mg, dextromethorphan 30 mg) orally under fasting conditions with 240 mL of warm water. Subjects were deprived of water for 1 h pre-dose and 1 h post-dose and of food for 4 h post-dose. A total of 8 mL of venous blood was collected at pre-dose and 0.25 h, 0.5 h, 1 h. 2 h, 3 h, 4 h, 6 h. 8 h, 10 h, 12 h, 24 h, 48 h, 72 h, 96 h, and 120 h post-dose for PK analysis. After completion of blood sampling on D6, postprandial administration of rivoceranib was conducted on D6 through D15 with the exception of D11 where subjects were fasted. Subjects were given standard diets 60 minutes pre-dose and completed the meal in 30 minutes. Subjects took 200 mg of rivoceranib with 240 mL of warm water at exactly 60 minutes after the start of the meal. Additionally, a single dose of Cooperstown Cocktail (in combination with rivoceranib) was orally administered in fasting conditions on D11 with 240 mL of warm water. The subject was deprived of water for 1 h pre-dose and 1 h post-dose and of food for 4 h post-dose. A total of 8 mL of venous blood was collected at pre-dose and at 0.25 h, 0.5 h, 1 h, 2 h, 3 h, 4 h, 6 h, 8 h, 10 h, 12 h, 24 h, 48 h, 72 h, 96 h, and 120 h post-dose. Subjects completed all pre-withdrawal examinations after PK blood sampling on D16.

Subjects were discharged after completing the PK blood collection and end-of-treatment (EOT) examinations on the morning of D16. Subjects then returned to the CRU for a safety follow-up visit on any one day between D21 and D25 to follow-up unresolved AEs/serious adverse events (SAEs) and to collect any new AEs/SAEs and any concomitant medications used. AEs that occurred during the trial were followed until they resolved, returned to the baseline level, or stabilized to a level satisfactory to the investigator.

### Number of Subjects

Planned: The study planned to enroll a minimum of 16 subjects and was required to end with at least 12 evaluable subjects.

Analyzed: A total of 18 subjects were enrolled; 15 subjects completed the study.

### Inclusion Criteria

1. Sign the written informed consent form (ICF) and fully understand the content, willing to comply with visits and procedures, and understand possible AEs before the trial starts.

2. A consent form for COVID-19 testing was sent electronically to potential subjects before scheduling screening. Testing was mandatory for all subjects. Subjects must be COVID-19 negative for inclusion.

3. Healthy male and female subjects ages 18 to 45 years old, inclusive, at time of signing ICF.

4. Male body weight ≥50 kg, female body weight ≥45 kg; body mass index (BMI) of ≥19 kg/m2 and ≤29 kg/m2.

5. No clinically significant abnormalities in medical history, general physical examination, vital signs, laboratory tests (hematology, urinalysis, blood chemistry) and 12-lead electrocardiogram (ECG) as judged by the investigator at screening and baseline (may be confirmed on repeat sampling).

6. Female subjects who are not pregnant or lactating and agree to maintain effective contraception from beginning of the study screening to 90 days after the last dose of the study drug:
a. A negative urine pregnancy test for women of childbearing potential at screening and a negative serum pregnancy test at baseline (positive urine pregnancy tests must be confirmed with a serum pregnancy test).
b. Both men and women of reproductive potential are willing and able to employ a highly effective method of birth control/contraception to prevent pregnancy. A highly effective method of contraception is defined as one that results in a low failure rate (i.e., less than 1% per year), when used consistently and correctly. Females of non-childbearing potential are defined as permanently sterile (i.e., due to hysterectomy, bilateral oophorectomy, or bilateral salpingectomy) or postmenopausal (defined as at least 12 months following cessation of menses without an alternative medical cause and serum follicle stimulating hormone (FSH) levels >25 IU/L).

7. Male subjects who are not surgically sterilized and are sexually active with a female partner of childbearing potential, must agree to use of a condom plus effective contraception for their female partner, throughout the study and for 90 days after last dose of the study drug. Sperm donation should be avoided for this same period.

8. Able and willing to abstain from caffeine- and xanthine-containing products, tobacco/nicotine-containing products, and alcohol for 5 days prior to baseline until PK blood sample (D16).

9. Able and willing to abstain from eating and drinking poppy seed-containing products and grapefruit-related fruits and juices (e.g., Seville oranges, pomelos) within 14 days prior to dosing until after collection of the final PK blood sample (D16).

10. Able and willing to abstain from strenuous exercise (heavy lifting, weight training, calisthenics, aerobics) within 7 days prior to dosing until after collection of the final PK blood sample (D16).

### Exclusion Criteria

1. Evidence or history of clinically significant hematological, renal, endocrine, pulmonary, gastrointestinal, cardiovascular, hepatic, neuropsychiatric, or allergic disease (including drug allergies, but excluding untreated, asymptomatic, seasonal allergies at the time of dosing); any condition possibly affecting drug absorption, distribution, metabolism, and excretion.

2. Had a severe infection, trauma, or major surgery within 4 weeks of screening; plan to have a surgery during the trial.

3. A past medical history of clinically significant cardiovascular conditions, including ECG abnormalities and arrhythmias; or QTc interval >450 msec for males, >470 msec for females or <300 msec; resting heart rate <45 or >100 beats per minute (BPM); respiratory rate <10 or >20 breaths/min at screening or baseline.

4. Supine systolic BP≥140 mmHg or <90 mmHg; diastolic BP≥90 mmHg or <50 mmHg on a single measurement (confirmed by a single repeat, if necessary).

5. History of immunodeficiency including seropositivity for HIV, or other acquired or congenital immune-deficient disease, or any active systemic viral infection requiring therapy (e.g., hepatitis B or C).

6. Subject has a history of Type 1 hypersensitivity to any medication.

7. Evidence of substance abuse or a history of substance abuse in the last 2 years.

8. Subject is positive for drugs of abuse (fentanyl, oxycodone, methamphetamines, opiates, methadone, cocaine, amphetamines, cannabinoids/synthetic cannabinoids/tetrahydrocannabinol (THC), tricyclic antidepressants, phencyclidine, barbiturates, benzodiazepines), cotinine or alcohol at screening and baseline; may be repeated once at the discretion of the investigator.

9. Consumption of foods and beverages containing poppy seeds, grapefruit, Seville oranges, star fruit, pomelos, or products containing these fruits from 14 days before baseline until discharge (D16).

10. Consumption of caffeine- and xanthine-containing foods and beverages from 5 days prior to baseline until discharge (D16).

11. History of severe hypoglycemia.

12. Subjects who are currently or have been smokers or users of tobacco or nicotine replacement products within 1 month prior to first admission to the CRU.

13. History of regular alcohol consumption in the past 3 months exceeding an average weekly intake of 14 standard drinks; 1 drink=5 ounces (150 mL) of wine or 12 ounces (360 mL) of beer or 1.5 ounces (45 mL) of hard liquor. Subject must be alcohol-free 72 hours prior to screening and baseline.

14. Use or intend to use any prescription medications/products other than hormone replacement therapy, oral, implantable, transdermal, injectable, or intrauterine contraceptives within 30 days prior to dosing, unless deemed acceptable by the Investigator (or designee).

15. Use or intend to use any nonprescription/over-the-counter medications, including vitamins, minerals, and phytotherapeutic-/herbal-/plant-derived preparations within 7 days prior to baseline, unless deemed acceptable by the Investigator.

16. Use or intend to use any medications/products known to alter drug absorption, metabolism, or elimination processes, including St. John's wort, within 30 days prior to dosing, unless deemed acceptable by the Investigator (or designee).

17. Use or intend to use slow-release medications/products considered to still be active within 14 days prior to baseline, unless deemed acceptable by the Investigator (or designee).

18. Participation in a clinical study involving administration of an investigational drug (new chemical entity) in the past 3 months or 5 half-lives prior to dosing, whichever is longer.

19. Require treatment with inhibitors of CYP3A, CYP2C9, CYP2D6. CYP2C19, or CYP1A2, with inducers of CYP3A or CYP1A2, or with rifampin (inducer of multiple CYPs) within 30 days prior to the first administration of the drug cocktail until discharge (D16).

20. Require treatment with substrates of CYP3A, CYP2C9, CYP2D6, CYP2C19, or CYP1A2 with narrow therapeutic indices within 14 days prior to the first administration of the drug cocktail until discharge (D16), at the discretion of the Investigator and by agreement with the sponsor, or clinical pharmacologist, or designee.

21. Require ongoing treatment with midazolam, warfarin, dextromethorphan, omeprazole, or caffeine, or require treatment with a drug contraindicated with midazolam, warfarin, dextromethorphan, omeprazole, or caffeine (per the prescribing label and Package Insert for each drug) within 14 days prior to the first administration of the drug cocktail until discharge (D16).

22. Blood donation or loss of more than 200 mL of blood within 1 month of dosing; or blood donation or loss of more than 400 mL of blood within 3 months; or received blood products within 8 weeks of dosing.

23. Any other major illness/condition that, in the Investigator's judgment, substantially increased the risk associated with the subject's participation in and completion of the study or could preclude the evaluation of the subject's response.

Removal of Subjects from Study Participation

Subjects had the right to withdraw consent and withdraw from the study or refuse to receive further treatment or examinations without withdrawing consent resulting in loss of follow-up (also considered withdrawal or drop-out). The reason for such withdrawal was documented. Subjects who were withdrawn for reasons not related to study drug could be replaced following discussion between the Investigator and the Sponsor. The Investigator could withdraw an enrolled subject from the trial per the following criteria:
The Investigator considered it necessary to discontinue the trial from a medical ethics perspective.
The subject had an SAE and was deemed not suitable to continue the study.
The Investigator judged that withdrawal from the study was more beneficial to the subject.
Poor compliance, including the following:
   The subject did not take the study drugs or undergo assessments as required.
   The subject used a drug or food affecting the results of safety assessment and PK study.
   The subject smoked or drank alcohol during the study.
   The subject had other behavior affecting study results.
Female subject became pregnancy during the study.
Other reasons for treatment discontinuation as determined by the Investigator.

### Test Product, Dose, and Mode of Administration:

Subjects were administered 200 mg of rivoceranib orally daily on D6 through D15 with 240 mL of warm water 60 minutes after the start of a meal, with the exception of D11 where rivoceranib was administered in a fasted state.

### Investigational Product(s):

Rivoceranib tablets (200 mg/tablet free base form for oral administration) were packaged in polyvinylidene chloride (PVDC), heat-sealed foil-laminated blister packs or in aluminum-aluminum cold form blisters.

### Duration of Treatment:

Subjects were treated with rivoceranib for ten days (D6-D15).

### Reference Therapy, Dose, and Mode of Administration:

Subjects were administered a single dose of Cooperstown 5+1 Cocktail orally under fasting conditions with 240 mL of warm water on D1 and D11. The Cooperstown 5+1 Cocktail includes midazolam (5 mg), warfarin (10 mg), vitamin K (10 mg), caffeine (200 mg), omeprazole (40 mg), and dextromethorphan (30 mg).

### Pharmacokinetic Assessments:

(a) Primary PK parameters of the substrates of cytochrome P450 enzymes (CYP1A2, CYP2C9. CYP2C19. CYP2D6, CYP3A4/5) assessed included Cₘₐₓ, AUC₀₋ₜ, and AUC_{0-∞} (if applicable).
(b) Secondary PK parameters of the substrates of cytochrome P450 enzymes (CYP1A2, CYP2C9. CYP2C19. CYP2D6, CYP3A4/5) assessed included Tₘₐₓ, t_{1/2}, Cl/F, and Vz/F

### General Statistical Methodology

Results were based on descriptive statistics and inferential statistics (i.e., confidence intervals [CIs]). Unless otherwise stated, continuous variables were summarized via sample size, arithmetic mean, standard deviation (SD), standard error (SE), arithmetic coefficient of variation (CV %), median, minimum, and maximum. Additional summaries included geometric mean and geometric CV %. CIs were provided for selected analyses of PK parameters. Categorical variables were summarized via frequency counts and percentages. For the calculation of summary statistics and statistical analysis, unrounded data was used.

### Pharmacokinetic Analysis

Plasma concentrations of the following analytes were listed and summarized by the scheduled sampling time using descriptive statistics: rivoceranib, midazolam, 1-hydroxymidazolam, R-warfarin, S-warfarin, dextromethorphan, dextrorphan, omeprazole, 5-hydroxyomeprazole, caffeine, and paraxanthine. Mean and median drug concentration-time curves were plotted as well as drug concentration-time curves for individual subjects.

PK parameters were listed for each subject in the PK parameter analysis set, and descriptive statistics (n, mean. SD, arithmetic CV %, geometric mean, geometric CV %, median, min and max) on each PK parameter were computed. The 90% CI on the geometric mean was also provided. These summaries were reported by treatment group.

### Prior and Concomitant Medications

Medications/products taken within 28 days before the first dose of study drug administration were documented as a prior treatment. Medications/products taken after the first dose of study treatment were documented as concomitant treatments. Concomitant medication documentation was recorded at each visit, continuing through the last Safety Follow-Up (visit on any one day between D21 and D25 and phone call 30 days [±2 days] after last dose of study drug). All prior and concomitant medications were listed.

### Study Timeline

This study was an open-label, fixed-sequence, crossover, drug interaction study. The overall design schema is shown in FIG. 1 with Table 1 listing a detailed breakdown.

### Screening Period (Day −21 to Day −3):

The following were assessed/collected:
Written informed consent
Written COVID-19 testing consent
Demographics: including sex, age, race/ethnicity
Medical and surgical history
Smoking and alcohol history
Height, weight, and body mass index (BMI)
Physical examination
Supine vital signs: body temperature, respiratory rate, heart rate, and blood pressure (BP) after the subject had rested quietly for at least 5 minutes
12-lead ECG obtained after the subject had rested quietly for at least 10 minutes (could be performed two additional times if abnormal)
Laboratory tests (hematology, urinalysis, blood biochemistry, coagulation)
Breath alcohol/urine drug screening
Viral serology: COVID-19 (RAPID test, reverse transcriptase polymerase chain reaction [RT-PCR] enzyme-linked immunosorbent assay [ELISA]), hepatitis B surface antigen (HBsAg), hepatitis C virus (HCV), and human immunodeficiency virus (HIV) antibody
Pregnancy test: a urine pregnancy test was performed at screening for women of childbearing potential. A serum pregnancy test was required to confirm any positive urine tests.
Confirmation of inclusion/exclusion criteria
Prior/concomitant medication
AE/serious adverse event (SAE) documentation

### Baseline Period (Day −2 to Day −1):

The following were assessed/collected:
Physical examination
Weight (fasting) and BMI. If weight and BMI were captured within 7 days of Baseline, they did not need to be repeated.
Supine vital signs: body temperature, respiratory rate, heart rate, and BP after the subject had rested quietly for at least 5 minutes
12-lead ECG obtained after the subject had rested quietly for at least 10 minutes (could be performed two additional times if abnormal)
Laboratory tests (hematology, urinalysis, blood biochemistry, coagulation): if the test was completed within 7 days before enrollment, the test was not required during the baseline period
Breath alcohol/urine drug screening
COVID-19 testing (RAPID test, RT-PCR ELISA)
Pregnancy test: a serum pregnancy test was performed at baseline (D−2) for women of childbearing potential.
Confirmation of inclusion/exclusion criteria
Prior/concomitant medication
AE/SAE documentation

### Treatment Period (Day 1 to Day 16):

Subjects were confined in the clinical research unit (CRU) for 17 days and discharged home after completion of end-of-treatment (EOT) assessments on D16. On D1, each subject was administered Cooperstown Cocktail orally as described below. Venous blood was collected at the timepoints outlined in Table 1. After completion of blood sampling on D6, postprandial administration of rivoceranib was conducted on D6 through D15 with the exception of Day 11 when subjects were fasted. Additionally, a single dose of Cooperstown Cocktail (in combination with rivoceranib) was orally administered under fasting conditions on D11. Venous blood was collected at the timepoints outlined in Table 1.

The Following were Performed Assessed on D1 Through D5:
Administration of Cooperstown Cocktail (midazolam 2 mg, warfarin 10 mg/vitamin K 10 mg, caffeine 200 mg, omeprazole 40 mg, dextromethorphan 30 mg) orally under fasting conditions with 240 mL of warm water on D1
Physical examination: performed D1 prior to dosing
ELISA testing for COVID-19 on D2 and D5
PK blood sampling: 8 mL of venous blood was collected pre-dose and at 0.25 h. 0.5 h, 1 h, 2 h. 3 h. 4 h, 6 h, 8 h. 10 h, 12 h, 24 h, 48 h, 72 h, 96 h, and 120 h post-dose
Supine vital signs were measured after the subject had rested quietly for at least 10 minutes, at 1 h (±5 min) pre-dose, and 24 h (±30 min), 48 h (±30 min), 72 h (±30 min), and 96 h (±30 min) after last dose
12-Lead ECG was measured after the subject had rested quietly for at least 10 minutes at 4 h (±30 min) post-dose on D1 (could be performed two additional times if abnormal)
Laboratory tests (hematology, urinalysis, blood biochemistry, coagulation) performed at 6 h, 24 h and 48 h post-dose on D1. If samples were not collected at D2, they were collected pre-dose. If the test was completed within 7 days before first dosing day, the test was not required during Baseline period or pre-dose.
Prior/concomitant medication daily
AE/SAE documentation daily
   The Following were Performed/Assessed on D6 Through D16:
After completion of blood sampling on D6, postprandial administration of rivoceranib was conducted on D6 through D15 with the exception of D11 when subjects were fasted.
Administration of Cooperstown Cocktail (in combination with rivoceranib) orally under fasting conditions on D11 with 240 mL of warm water
PK blood sampling: 8 mL of venous blood was collected pre-dose and at 0.25 h, 0.5 h, 1 h, 2 h, 3 h, 4 h, 6 h, 8 h, 10 h, 12 h, 24 h, 48 h, 72 h, 96 h, and 120 h post-dose
Physical examination: performed D6 and D11 pre-dose, and D16
ELISA testing for COVID-19 on D8, D11. D14, and D16 (EOT)
On D6 through D15, supine vital signs were measured, after the subject had rested quietly for at least 5 minutes, at 1 h (±5 min) pre-dose, and 24 h (±30 min) after last dose and D16
12-Lead ECG was measured after the subject had rested quietly for at least 10 minutes at 4 h (±30 min) post-dose on D6 and D11. ECG was measured on D16 (could be performed two additional times if abnormal).
Laboratory tests (hematology, urinalysis, blood biochemistry, coagulation) performed at 6 h, 24 h and 48 h post-dose on D11. Laboratory tests were also performed at D16.
Prior/concomitant medication daily
AE/SAE documentation daily
   Safety Follow-Ups (Day 21 and Day 25; Phone Call 30±2 Days after last dose)

Subjects returned to the CRU for safety follow-up procedures on any one day between D21 and D25. The CRU called subjects 30 days (±2 days) after the last dose to follow-up on unresolved AEs/SAEs, to collect any new AEs/SAEs, and collect any concomitant medication used within 30 days after the last dose. AEs that occurred during the trial were followed until they resolved, returned to baseline, or stabilized to a level satisfactory to the investigator.

The Following were Performed/Assessed at the Day 19-23 Follow-Up:
Supine vital signs: body temperature, respiratory rate, heart rate, and BP after the subject had rested quietly for at least 5 minutes
12-lead ECG obtained after the subject had rested quietly for at least 10 minutes (could be performed two additional times if abnormal)
Laboratory tests (hematology, urinalysis, blood biochemistry, coagulation)
Prior/concomitant medication
AE/SAE documentation
RAPID testing for COVID-19
   The Following were Assessed on the Safety Follow-Up Call:
AE/SAE documentation
Prior/concomitant medication

TABLE 1
Study Schedule
Time

| | | | |
|---|---|---|---|
| Screening | | | Safety Follow- |
| Period | Baseline | Treatment, Observation, and PK Blood Sampling Period | Up |

Item

| | | | | | | | | | | | | | | | 30 |
|---|---|---|---|---|---|---|---|---|---|---|---|---|---|---|---|
| | | | | | | | | | | | | | | 1 day | (±2) |
| | | | | | | | | | | | | | | between | days |
| | | | | | | | | | | | | | | D21 | after |
| D -21 - | | | D2- | | | | D7- | | | | | | D16/ | and | last |
| D -3 | D -2 | D1 | D3 | D4 | D5 | D6 | D10 | D11 | D12 | D13 | D14 | D15 | EOT¹ | D25² | dose³ |

Dosing Days

| | | | | | | | |
|---|---|---|---|---|---|---|---|
| | | | Rivo | | | | |
| | | Rivo | + | | | | |
| CC/F | Rivo | NBD | CC/F | Rivo | Rivo | Rivo | Rivo |

Baseline Data

| | | | | | | | | | | | | | | | |
|---|---|---|---|---|---|---|---|---|---|---|---|---|---|---|---|
| Signing Informed | X | | | | | | | | | | | | | | |
| Consent Form | | | | | | | | | | | | | | | |
| Signing COVID-19 | X | | | | | | | | | | | | | | |
| Informed Consent | | | | | | | | | | | | | | | |
| Form | | | | | | | | | | | | | | | |
| Inclusion/Exclusion | X | X | | | | | | | | | | | | | |
| Criteria | | | | | | | | | | | | | | | |
| Demographics | X | | | | | | | | | | | | | | |
| Medical History | X | | | | | | | | | | | | | | |
| Alcohol and | X | | | | | | | | | | | | | | |
| Smoking History | | | | | | | | | | | | | | | |
| Physical⁴ | X | X | X | | | | X | | X | | | | | X | |
| Vital Signs⁵ | X | X | X | X | X | X | X | X | X | X | X | X | X | X | X |
| Height | X | | | | | | | | | | | | | | |
| Weight (Fasting) | X | X | | | | | | | | | | | | | |
| and BMI | | | | | | | | | | | | | | | |

Laboratory Testing

| | | | | | | | | | | | | | |
|---|---|---|---|---|---|---|---|---|---|---|---|---|---|
| COVID-19 testing⁶ | X | X | | X⁶ | X | | X⁶ | X | | | X | X | X |
| Hematology⁷ | X | X | X | X | | | | X | X | X | | X | X |
| Urinalysis⁸ | X | X | X | X | | | | X | X | X | | X | X |
| Blood | X | X | X | X | | | | X | X | X | | X | X |
| Biochemistry⁹ | | | | | | | | | | | | | |
| Coagulation¹⁰ | X | X | X | X | | | | X | X | X | | X | X |
| Infectious Disease | X | | | | | | | | | | | | |
| Screening¹¹ | | | | | | | | | | | | | |
| Drug Screening¹² | X | X | | | | | | | | | | | |
| Breath Alcohol | X | X | | | | | | | | | | | |
| Test¹² | | | | | | | | | | | | | |
| 12-Lcad ECG¹³ | X | X | X | | | X | | X | | | | X | X |
| Pregnancy Test¹⁴ | X | X | | | | | | | | | | | |

Study Drugs

| | | | | | | | | | | | | |
|---|---|---|---|---|---|---|---|---|---|---|---|---|
| Cooperstown | X | | | | | | X | | | | | |
| Cocktail | | | | | | | | | | | | |
| Administration | | | | | | | | | | | | |
| Rivoceranib | | | | | X | X | X | X | X | X | X | |
| Administration | | | | | | | | | | | | |
| PK Blood | X | X | X | X | X | | X | X | X | X | X | X |
| Sampling¹⁵ | | | | | | | | | | | | |

| | | | |
|---|---|---|---|
| Concomitant | X | X | X |
| Medication¹⁶ | | | |
| Adverse Event | X | X | X |
| Recording¹⁷ | | | |

Abbreviations: AE = adverse event; BMI = body mass index; CC/F = Cooperstown Cocktail/fasting; COVID-19 = coronavirus disease 2019; CRU = clinical research unit; ECG = electrocardiogram; EOT = end-of-treatment; FSH = follicle-stimulating hormone; HbsAg = Hepatitis B surface antigen; HCV = Hepatitis C virus; HIV = human immunodeficiency virus; ICF = informed consent form; NBD = no blood draw; PK = pharmacokinetics; Rivo = rivoceranib; SAE = serious adverse event
1. End-of-Treatment (Physical Examination, Vital Signs, Hematology, Urinalysis, Blood Biochemistry, Coagulation, 12-Lead ECG, PK Blood Sampling, Concomitant Medication, AE Recording, and Covid-19 testing) assessments completed, if possible. These assessments were also completed at Early Termination.
2. Safety Follow-up Visit: Subjects returned to the CRU for a safety follow-up visit on any 1 day between D21 to D25 to perform follow-up safety blood draws and examinations, to collect unresolved AEs/SAEs and to collect any new AEs/SAEs.
3. Safety Follow-up phone call completed 30 (±2) days after last dose. CRU called subjects to follow-up on any unresolved AEs/SAEs, to collect any new AFs/SAEs, and collect any concomitant medications used.
4. Physical examination: Screening period, Baseline, D1, D6, D11, D16; examination of main body systems (head and face, skin system, lymph nodes, eyes, ears, nose, throat, oral cavity, respiratory system, cardiovascular system, abdomen, urogenital system, musculoskeletal system, nervous system, and mental state). Physical examinations on D1, D6 and D11 were completed prior to dosing.
5. Vital signs (supine): Screening period, Baseline. D1 1 h pre-dose, and 24 h, 48 h, 72 h. and 96 h post-dose; D6 through D15 at 1 h pre-dose, and 24 h post last dose (D16); pulse, respiratory rate, body temperature, and blood pressure. NOTE: Blood collection occurred after vital signs were collected. Supine vital signs were measured after the subject had rested quietly for at least 5 minutes.
6. Subjects were admitted to the CRU on D -2 and tested for COVID-19 (RAPID test, reverse transcriptase polymerase chain reaction [RT-PCR] enzyme-linked immunosorbent assay [ELISA]). COVID-19 RAPID test was performed prior to undergoing Screening procedures. ELISA was used to test for COVID-19 every 3 days during confinement (D -2, D2, D5, D8, D11, D14, and D 16[EOT]). COVID-19 RAPID test was also performed at Safety Follow-up Visit.
7. Hematology: Screening period, Baseline, performed at 6 h, 24 h and 48 h post-dose on D1; performed at 6 h, 24 h and 48 h post-dose on D11, and D16; white blood cell count (WBC), red blood cell count (RBC), platelet count (PLT), hemoglobin (Hb), hematocrit (HCT), % lymphocytes (L %), % neutrophils (N %), and % monocytes (M %). If samples were NOT collected at D -2, they needed to be collected at pre-dose timepoint. If the test was completed within 7 days before first dosing day, the test was not required during Baseline period.
8. Urinalysis: Screening period, Baseline, performed at 6 h, 24 h and 48 h post-dose on D1, performed at 6 h, 24 h and 48 h post-dose on D11; and D16; urine protein (U-PRO), urine glucose (U-GLU), urine white blood cell (U-WBC), urine ketone (U-KET), and urine red blood cell (U-RBC). If samples were NOT collected at D -2, they needed to be collected at pre-dose timepoint. If the test was completed within 7 days before first dosing day, the test is not required pre-dose during Baseline period.
9. Blood biochemistry: Screening period, Baseline, performed at 6 h, 24 h and 48 h post-dose on D1, performed at 6 h, 24 h and 48 h post-dose on D11; and D16; aspartate transaminase (AST), alanine transaminase (ALT), alkaline phosphatase (ALP), lactate dehydrogenase (LDH), total bilirubin (TBIL), total protein (TP), albumin (ALB), triglyceride (TG), total cholesterol (TC), uric acid (UA), Na, K, Cl, Ca, P, Mg, Urea, creatinine (Cr), and fasting blood glucose (GLU). If samples were NOT collected at D -2, they needed to be collected at pre-dose timepoint. If the test was completed within 7 days before first dosing day, the test was not required pre-dose during Baseline period.
10. Coagulation: Screening period, Baseline, performed at 6 h, 24 h and 48 h post-dose on D1, performed at 6 h, 24 h and 48 h post-dose on D11; and D16; prothrombin time (PT), activated partial thromboplastin time (APTT), and international normalized ratio (INR). If samples were NOT collected at D -2, they needed to be collected at pre-dose timepoint. If the test was completed within 7 days before first dosing day, the test was not required pre-dose during Baseline period.
11. Infectious disease screening: Screening period; HbsAg, HCV antibody, HIV antibody, and syphilis antibody.
12. Drug screening and breath alcohol screening: Screening period; Baseline; a 5-panel drug test was performed during the screening period.
13. 12-Lead ECGs were measured after the subject had rested quietly for at least 10 minutes in a supine position. 12-Lead ECG: Screening period, Baseline, D1 (4 h ± 30 min post-dose), D6 (4 h ± 30 min post-dose), D11 (4 h ± 30 min post-dose) and D16. If the results of 12-lead ECG were abnormal, 2 additional tests were performed as determined by the investigator (5 minutes apart, the QTc interval was required to be indicated).
14. Pregnancy test: Screening period and Baseline. A urine pregnancy test was performed in women of childbearing potential only. If the results were positive, a serum pregnancy test was performed for confirmation. A serum pregnancy test was performed at baseline. An FSH test was performed on all women of non-childbearing potential. Blood samples for analysis of Cooperstown Cocktail and metabolites were collected on D1 and D11 pre-dose, at 0.25 h, 0.5 h, 1 h, 2 h, 3 h, 4 h, 6 h, 8 h, 10 h, 12 h, 24 h, 48 h, 72 h, 96 h and 120 hrs post-dose. Blood samples collected at D11 pre-dose were used for rivoceranib quantification. The allowed sampling window for PK blood samples was as follows: within 30 minutes prior to dosing for the pre-dose sample timepoint; ±2 minutes for sampling timepoints within the first 0.5 h; = 5 minutes for sampling timepoints >0.5 and <4 h; ±10 minutes for sampling timepoints ≥4h and ≤12 h ; and ±30 minutes for the sampling timepoints >12 h.
16. Concomitant medication: All concomitant medications and treatments from 14 days before study treatment through the safety follow-up period were documented.
17. Adverse event recording: All AEs starting from signing the ICF were documented until the end of the study.

### Pharmacokinetic Variables

Pharmacokinetic (PK) samples were collected from all participants for measurement of plasma concentrations of rivoceranib, midazolam, 1-hydroxymidazolam, R-warfarin, S-warfarin, dextromethorphan, dextrorphan, omeprazole, 5-hydroxyomeprazole, caffeine, and paraxanthine at the time points indicated herein. Using the concentration data, non-compartmental analysis (NCA) was utilized to estimate the following PK parameters for each subject for each treatment group. Table 2 provides a summary of the PK variables.

TABLE 2
Summary of PK Variables

| PK | |
|---|---|
| Parameter | Definition |
| Cₘₐₓ | Maximum observed plasma concentration |
| tₘₐₓ | Time of maximum observed plasma concentration; if it |
| | occurs at more than one time point, Tₘₐₓ is defined as the |
| | first time point with this valuc |
| AUC₀₋ₜ | Cumulative area under the plasma concentration time curve |
| | calculated from 0 to the last measured plasma concentration |
| | using the linear up and log down method. |
| AUC_{0-∞} | Area under the plasma concentration time curve |
| | extrapolated to infinity, calculated as AUCₒ₋ₜ + C_{LQC}/λ_{z}, |
| | where C_{LQC} is the measured concentration at time T_{LQC} |
| λ_{z} | Apparent elimination rate constant, estimated by linear |
| | regression of the terminal linear portion of the log |
| | concentration versus time curve |
| t_{1/2} | Terminal elimination half-life, calculated as ln(2)/λ_{z} |
| t_{lag} | Signifies the delay between the time of dosing and time of |
| | appearance of concentration in the sampling compartment. |
| tₗₐₛₜ | Time of last measurable concentration |
| Cₗₐₛₜ | Last measurable concentration (above the quantification |
| | limit) |
| Vz/F | Steady-state distribution volume based on the last observed |
| | concentration |
| CL/F | Clearance |

### Drug Concentration Measurements and Time Points

Plasma samples (8 mL) were collected at each of the following time points and drug concentration was assessed for midazolam, 1-hydroxymidazolam (metabolite of midazolam), R-warfarin, S-warfarin, dextromethorphan, dextrorphan (metabolite of dextromethorphan), omeprazole, 5-hydroxyomeprazole (metabolite of omeprazole), caffeine, and paraxanthine (des-methyl metabolite of caffeine):
Beginning on D1: pre-dose and at 0.25 h, 0.5 h, 1 h, 2 h, 3 h, 4 h, 6 h, 8 h, 10 h, 12 h, 24 h, 48 h, 72 h, 96 h, and 120 h post-dose.
Beginning on D11: pre-dose and at 0.25 h, 0.5 h, 1 h, 2 h, 3 h, 4 h. 6 h, 8 h, 10 h, 12 h, 24 h, 48 h, 72 h, 96 h, and 120 h post-dose.

Rivoceranib concentration was measured on Day 11 pre-dose. Plasma samples for rivoceranib were analyzed by liquid-liquid extraction and LC-MS/MS. The method has been validated for the quantification of rivoceranib from 0.500 ng/mL to 500 ng/mL.

### Results

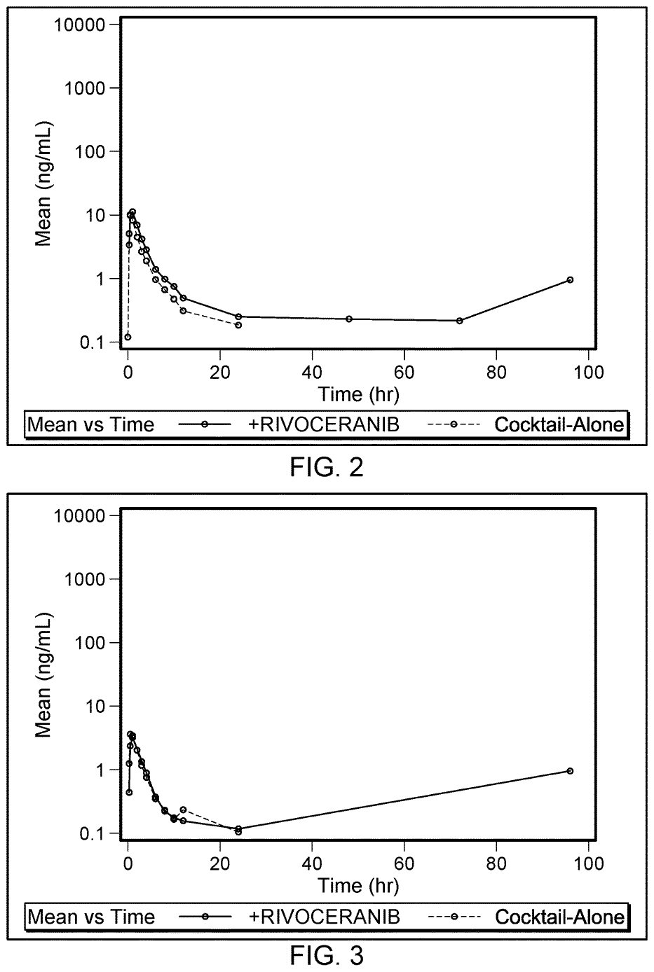

All cytochrome P-450 enzymes interrogated by the Cooperstown 5+1 cocktail were modulated by rivoceranib. The exposure results for Cooperstown 5+1 cocktail and Cooperstown 5+1 cocktail with coadministration with rivoceranib appear in FIGS. 2-11 and pharmacokinetics summarized in Tables 3-12.

Coadministration with rivoceranib caused inhibition of CYP3A4/A5, as evidenced by an increase in AUC_{0-∞} of 65% and Cₘₐₓ by 16% for midazolam (a CYP3A4/5 substrate in the Cooperstown 5+1 cocktail) following coadministration with rivoceranib (see FIG. 2 and Table 3).

TABLE 3
Change in PK parameters for Midazolam (N = 18)

| | | | (Cooperstown Cocktail + | | (Cooperstown Cocktail + | |
|---|---|---|---|---|---|---|
| | | | Rivoceranib)- | | Rivoceranib)/ | |
| | | | Cooperstown Cocktail | | Cooperstown Cocktail | |
| | Geometric LS Means | | Alone | | Alone | |
| | Cooperstown | Cooperstown | Geometric | | Geometric | |
| | Cocktail | Cocktail + | Mean | | Mean | |
| Parameter | Alone | Rivoceranib | Difference | 90% CI | Ratio | 90% CI |
| Cₘₐₓ | 2.2907 | 2.4313 | 0.1405 | (0.0095, | 1.1509 | (1.0095, |
| | | | | 0.2716) | | 1.3121) |
| AUC₍₀₋ₜ₎ | 3.1707 | 3.6136 | 0.4430 | (0.2780, | 1.5573 | (1.3205, |
| | | | | 0.6080) | | 1.8367) |
| AUC_{(0-∞)} | 3.2273 | 3.5955 | 0.3682 | (0.2416, | 1.4451 | (1.2732, |
| | | | | 0.4948) | | 1.6402) |

That 1-hydroxymidazolam was detected (FIG. 3 and Table 4) was consistent with metabolism of midazolam by the CYP3A subfamily (Perloff, M. D., et al, 2000, J. Pharmacol. Exp. Ther. 292(2), 618-628).

TABLE 4
Change in PK parameters for 1-hydroxymidazolam (N = 18)

| | | | (Cooperstown Cocktail + | | (Cooperstown Cocktail + | |
|---|---|---|---|---|---|---|
| | | | Rivoceranib)- | | Rivoceranib)/ | |
| | | | Cooperstown Cocktail | | Cooperstown Cocktail | |
| | Geometric LS Means | | Alone | | Alone | |
| | Cooperstown | Cooperstown | Geometric | | Geometric | |
| | Cocktail | Cocktail + | Mean | | Mean | |
| Parameter | Alone | Rivoceranib | Difference | 90% CI | Ratio | 90% CI |
| Cₘₐₓ | 1.2859 | 1.1708 | −0.1150 | (−0.3176, | 0.8913 | (0.7279, |
| | | | | 0.0875) | | 1.0915) |
| AUC₍₀₋ₜ₎ | 2.1534 | 2.3867 | 0.2332 | (0.0109, | 1.2627 | (1.0109, |
| | | | | 0.4555) | | 1.5770) |
| AUC_{(0-∞)} | 2.2138 | 2.3408 | 0.1271 | (0.0257, | 1.1355 | (1.0260, |
| | | | | 0.2283) | | 1.2565) |

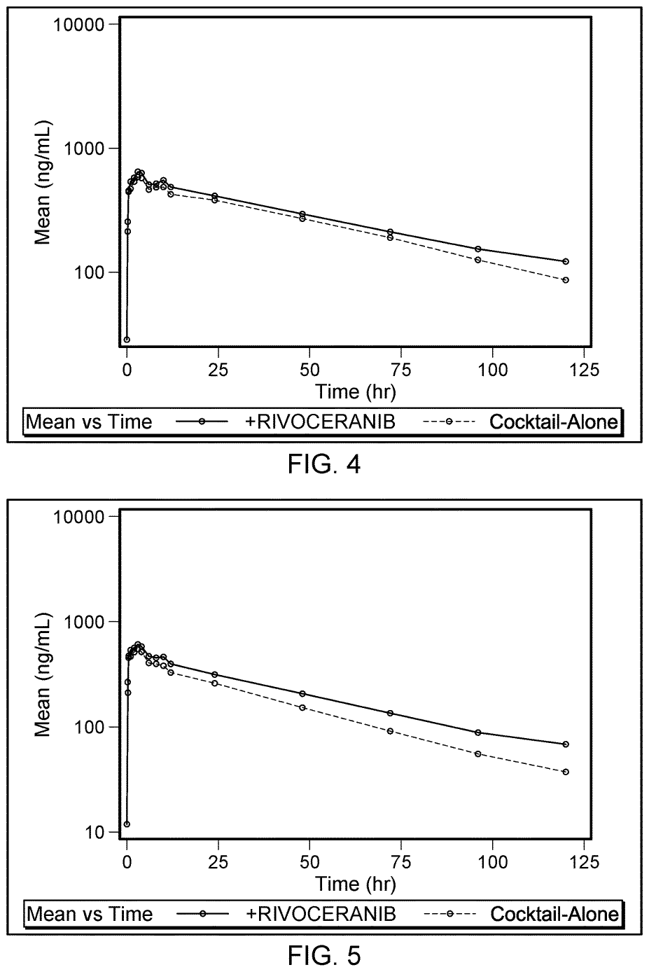

The levels of R-warfarin (which is in the Cooperstown 5+1 cocktail but not metabolized by CYP2C9) were relatively unaffected by coadministration with rivoceranib (FIG. 4 and Table 5).

TABLE 5
Change in PK parameters for R-warfarin (N = 18)

| | | | (Cooperstown Cocktail + | | (Cooperstown Cocktail + | |
|---|---|---|---|---|---|---|
| | | | Rivoceranib)- | | Rivoceranib)/ | |
| | | | Cooperstown Cocktail | | Cooperstown Cocktail | |
| | Geometric LS Means | | Alone | | Alone | |
| | Cooperstown | Cooperstown | Geometric | | Geometric | |
| | Cocktail | Cocktail + | Mean | | Mean | |
| Parameter | Alone | Rivoceranib | Difference | 90% CI | Ratio | 90% CI |
| Cₘₐₓ | 6.4888 | 6.5703 | 0.0815 | (0.0099, | 1.0849 | (1.0099, |
| | | | | 0.1531) | | 1.1655) |
| AUC₍₀₋ₜ₎ | 10.2738 | 10.3226 | 0.0487 | (−0.1163, | 1.0499 | (0.8902, |
| | | | | 0.2137) | | 1.2383) |
| AUC_{(0-∞)} | 10.4394 | 10.5244 | 0.0850 | (−0.0014, | 1.0887 | (0.9986, |
| | | | | 0.1714) | | 1.1869) |

A modest increase in S-warfarin (a CYP2C9 substrate in the Cooperstown 5+1 cocktail) AUC_{0-∞}, of 28% and Cmax of 5% with coadministration with rivoceranib point to inhibition of CYP2C9 by rivoceranib (FIG. 5 and Table 6).

TABLE 6
Change in PK parameters for S-warfarin (N = 18)

| | | | (Cooperstown Cocktail + | | (Cooperstown Cocktail + | |
|---|---|---|---|---|---|---|
| | | | Rivoceranib)- | | Rivoceranib)/ | |
| | | | Cooperstown Cocktail | | Cooperstown Cocktail | |
| | Geometric LS Means | | Alone | | Alone | |
| | Cooperstown | Cooperstown | Geometric | | Geometric | |
| | Cocktail | Cocktail + | Mean | | Mean | |
| Parameter | Alone | Rivoceranib | Difference | 90% CI | Ratio | 90% CI |
| Cₘₐₓ | 6.4628 | 6.5148 | 0.0520 | (−0.0450, | 1.0534 | (0.9560, |
| | | | | 0.1490) | | 1.1607) |
| AUC₍₀₋ₜ₎ | 9.8190 | 10.0033 | 0.1842 | (0.0316, | 1.2023 | (1.0321, |
| | | | | 0.3368) | | 1.4005) |
| AUC_{(0-∞)} | 9.9168 | 10.2200 | 0.3032 | (0.2500, | 1.3542 | (1.2840, |
| | | | | 0.3565) | | 1.4283) |

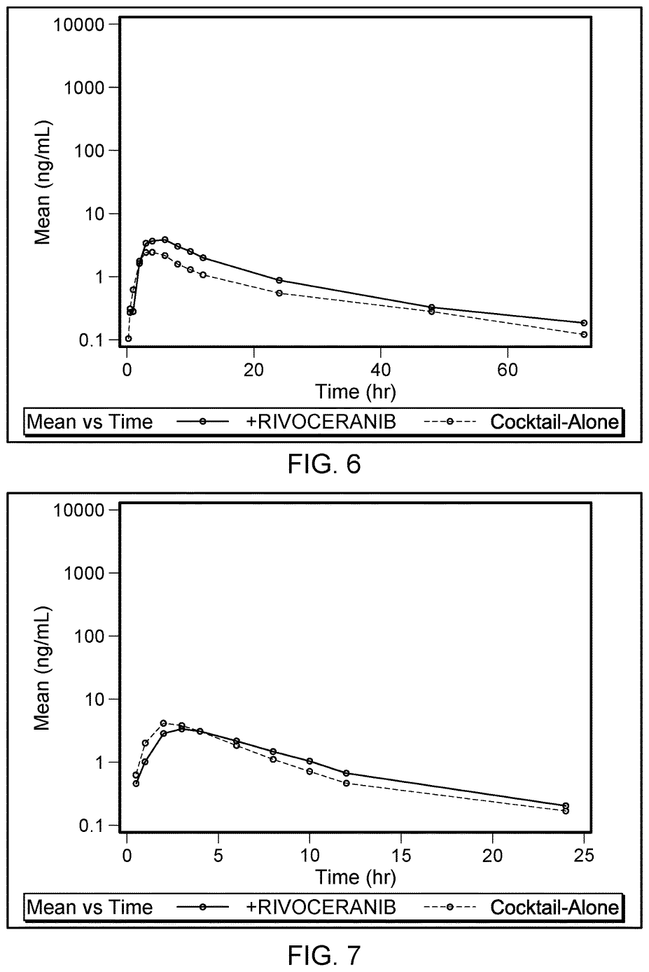

Coadministration of rivoceranib with dextromethorphan (a CYP2D6 substrate in the Cooperstown 5+1 cocktail) increased the dextromethorphan AUC_{0-∞} by 144% and Cmax by 70% relative to administration of only Cooperstown 5+1 cocktail (see FIG. 6 and Table 7), which is consistent with moderate inhibition of CYP2D6 by rivoceranib.

TABLE 7
Change in PK parameters for Dextromethorphan (N = 18)

| | | | (Cooperstown Cocktail + | | (Cooperstown Cocktail + | |
|---|---|---|---|---|---|---|
| | | | Rivoceranib)- | | Rivoceranib)/ | |
| | | | Cooperstown Cocktail | | Cooperstown Cocktail | |
| | Geometric LS Means | | Alone | | Alone | |
| | Cooperstown | Cooperstown | Geometric | | Geometric | |
| | Cocktail | Cocktail + | Mean | | Mean | |
| Parameter | Alone | Rivoceranib | Difference | 90% CI | Ratio | 90% CI |
| Cₘₐₓ | 0.4718 | 1.1156 | 0.6439 | (0.3378, | 1.9039 | (1.4019, |
| | | | | 0.9500) | | 2.5856) |
| AUC₍₀₋ₜ₎ | 2.4840 | 3.4181 | 0.9341 | (0.6206, | 2.5448 | (1.8601, |
| | | | | 1.2475) | | 3.4815) |
| AUC_{(0-∞)} | 3.0035 | 3.9837 | 0.9802 | (0.6937, | 2.6650 | (2.0010, |
| | | | | 1.2667) | | 3.5493) |

That dextrorphan (produced by O-demethylation of dextromethorphan by CYP2D6) was detected indicated CYP2D6-mediated metabolism (FIG. 7, and Table 8).

TABLE 8
Change in PK parameters for Dextrorphan (N = 18)

| | | | (Cooperstown Cocktail + | | (Cooperstown Cocktail + | |
|---|---|---|---|---|---|---|
| | | | Rivoceranib)- | | Rivoceranib)/ | |
| | | | Cooperstown Cocktail | | Cooperstown Cocktail | |
| | Geometric LS Means | | Alone | | Alone | |
| | Cooperstown | Cooperstown | Geometric | | Geometric | |
| | Cocktail | Cocktail + | Mean | | Mean | |
| Parameter | Alone | Rivoceranib | Difference | 90% CI | Ratio | 90% CI |
| Cₘₐₓ | 1.3542 | 1.2432 | −0.1109 | (−0.2944, | 0.8950 | (0.7450, |
| | | | | 0.0726) | | 1.0753) |
| AUC₍₀₋ₜ₎ | 3.0150 | 3.1556 | 0.1406 | (−0.0107, | 1.1509 | (0.9894, |
| | | | | 0.2918) | | 1.3389) |
| AUC_{(0-∞)} | 3.1245 | 3.2292 | 0.1047 | (−0.0295, | 1.1104 | (0.9709, |
| | | | | 0.2389) | | 1.2699) |

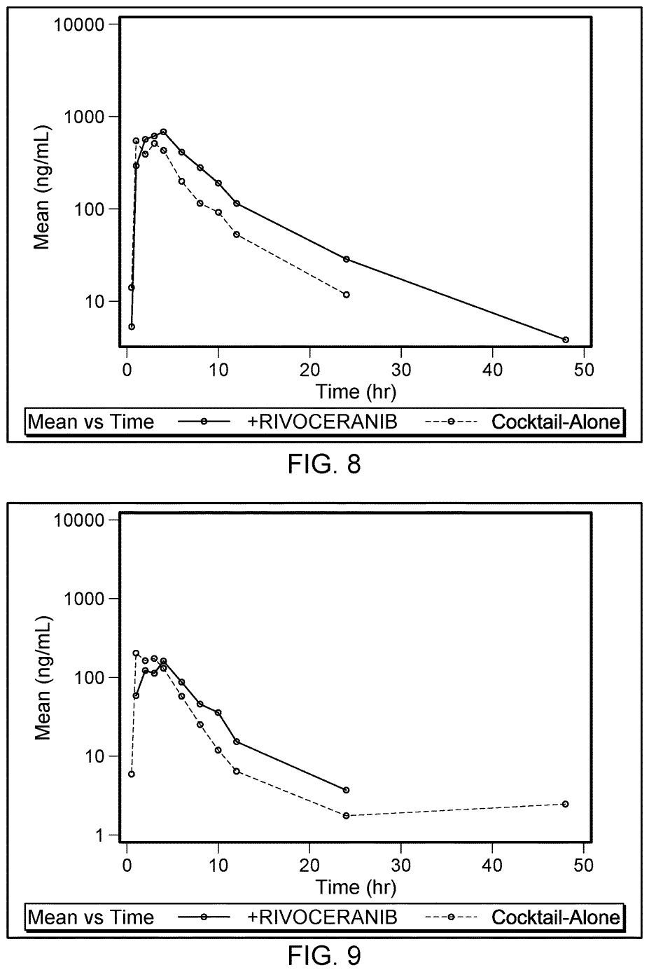

Coadministration of rivoceranib with omeprazole (a CYP2C19 substrate in the Cooperstown 5+1 cocktail) caused an increase in AUC_{0-∞} by 77% and an increase in Cmax increase by 62% (see FIG. 8 and Table 9), consistent with inhibition of CYP2C19.

TABLE 9
Change in PK parameters for Omeprazole (N = 18)

| | | | (Cooperstown Cocktail + | | (Cooperstown Cocktail + | |
|---|---|---|---|---|---|---|
| | | | Rivoceranib)- | | Rivoceranib)/ | |
| | | | Cooperstown Cocktail | | Cooperstown Cocktail | |
| | Geometric LS Means | | Alone | | Alone | |
| | Cooperstown | Cooperstown | Geometric | | Geometric | |
| | Cocktail | Cocktail + | Mean | | Mean | |
| Parameter | Alone | Rivoceranib | Difference | 90% CI | Ratio | 90% CI |
| Cₘₐₓ | 6.3720 | 6.8653 | 0.4933 | (0.2463, | 1.6377 | (1.2793, |
| | | | | 0.7403) | | 2.0966) |
| AUC₍₀₋ₜ₎ | 7.3711 | 8.2093 | 0.8382 | (0.6267, | 2.3123 | (1.8713, |
| | | | | 1.0498) | | 2.8572) |
| AUC_{(0-∞)} | 7.5365 | 8.2595 | 0.7233 | (0.4952, | 2.0612 | (1.6408, |
| | | | | 0.9514) | | 2.5894) |

That 5-hydroxyomeprazole was detected indicated CYP2C19-mediated metabolism of omeprazole (FIG. 9 and Table 10).

TABLE 10
Change in PK parameters for 5-hydroxyomeprazole (N = 18)

| | | | (Cooperstown Cocktail + | | (Cooperstown Cocktail + | |
|---|---|---|---|---|---|---|
| | | | Rivoceranib)- | | Rivoceranib)/ | |
| | | | Cooperstown Cocktail | | Cooperstown Cocktail | |
| | Geometric LS Means | | Alone | | Alone | |
| | Cooperstown | Cooperstown | Geometric | | Geometric | |
| | Cocktail | Cocktail + | Mean | | Mean | |
| Parameter | Alone | Rivoceranib | Difference | 90% CI | Ratio | 90% CI |
| Cₘₐₓ | 5.3867 | 5.1680 | −0.2187 | (−0.3771, | 0.8035 | (0.6858, |
| | | | | −0.0603) | | 0.9414) |
| AUC₍₀₋ₜ₎ | 6.6476 | 6.7369 | 0.0893 | (0.0262, | 1.0934 | (1.0265, |
| | | | | 0.1525) | | 1.1647) |
| AUC_{(0-∞)} | 6.6678 | 6.7462 | 0.0784 | (0.0112, | 1.0816 | (1.0112, |
| | | | | 0.1457) | | 1.1568) |

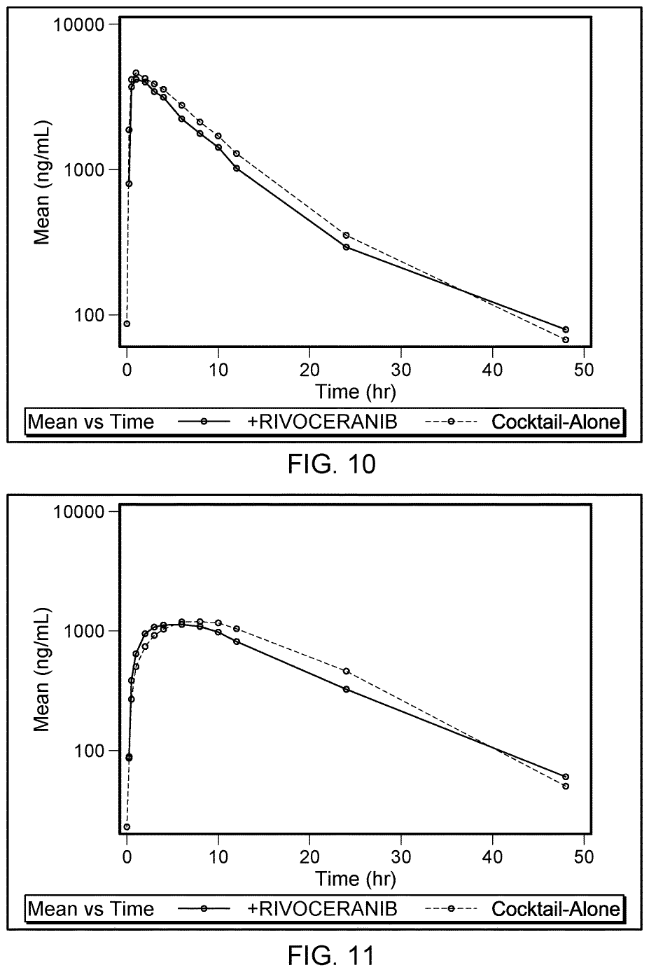

Rivoceranib appears to cause weak induction of CYP1A2, as evidenced by a slight decreased in the AUC_{0-∞} by 20% and Cmax by 7% for caffeine (a CYP1A2 substrate in the Cooperstown 5+1 cocktail) following coadministration with rivoceranib (FIG. 10 and Table 11).

TABLE 11
Change in PK parameters for Caffeine (N = 18)

| | | | (Cooperstown Cocktail + | | (Cooperstown Cocktail + | |
|---|---|---|---|---|---|---|
| | | | Rivoceranib)- | | Rivoceranib)/ | |
| | | | Cooperstown Cocktail | | Cooperstown Cocktail | |
| | Geometric LS Means | | Alone | | Alone | |
| | Cooperstown | Cooperstown | Geometric | | Geometric | |
| | Cocktail | Cocktail + | Mean | | Mean | |
| Parameter | Alone | Rivoceranib | Difference | 90% CI | Ratio | 90% CI |
| Cₘₐₓ | 8.4984 | 8.4258 | −0.0726 | (−0.1609, | 0.9300 | (0.8514, |
| | | | | 0.0157) | | 1.0159) |
| AUC₍₀₋ₜ₎ | 10.6274 | 10.4128 | −0.2146 | (−0.3397, | 0.8069 | (0.7120, |
| | | | | −0.0895) | | 0.9144) |
| AUC_{(0-∞)} | 10.6463 | 10.4425 | −0.2038 | (−0.3219, | 0.8156 | (0.7248, |
| | | | | −0.0858) | | 0.9178) |

The fact that paraxanthine was detected (FIG. 11 and Table 12) is consistent with metabolism of caffeine by CYP1A2, as CYP1A2 alone is responsible for caffeine 3-demethylation to produce paraxanthine (Gu, Lie, et al., Pharmacogenetics 1992, v2 (2) 73-77).

TABLE 12
Change in PK parameters for Paraxanthine (N = 18)

| | | | (Cooperstown Cocktail + | | (Cooperstown Cocktail + | |
|---|---|---|---|---|---|---|
| | | | Rivoceranib)- | | Rivoceranib)/ | |
| | | | Cooperstown Cocktail | | Cooperstown Cocktail | |
| | Geometric LS Means | | Alone | | Alone | |
| | Cooperstown | Cooperstown | Geometric | | Geometric | |
| | Cocktail | Cocktail + | Mean | | Mean | |
| Parameter | Alone | Rivoceranib | Difference | 90% CI | Ratio | 90% CI |
| Cₘₐₓ | 7.1157 | 7.1076 | −0.0080 | (−0.0873, | 0.9920 | (0.9164, |
| | | | | 0.0712) | | 1.0738) |
| AUC₍₀₋ₜ₎ | 10.0780 | 9.8707 | −0.2073 | (−0.3214, | 0.8128 | (0.7252, |
| | | | | −0.0933) | | 0.9109) |
| AUC_{(0-∞)} | 10.1025 | 9.9579 | −0.1446 | (−0.2295, | 0.8654 | (0.7949, |
| | | | | −0.0597) | | 0.9420) |

From the results presented in Example 1 it can be seen that rivoceranib is a mild inhibitor of the CYP3A subfamily (CYP3A4/A5), CYP2C9, and CYP2C19, and a more robust inhibitor of CYP2D6. Rivoceranib appears to be a weak inducer of CYP1 A2. These conclusions are supported by the following PK results:
(i) Coadministration of rivoceranib 200 mg once daily with oral midazolam (a sensitive CYP3A4/5 substrate) increased the midazolam AUC_{0-∞} by 65% and Cₘₐₓ by 16%;
(ii) Coadministration of rivoceranib 200 mg once daily with oral S-warfarin (a sensitive CYP2C9 substrate) increased the warfarin AUC_{0-∞} by 28% and Cₘₐₓ by 5%;
(iii) Coadministration of rivoceranib 200 mg once daily with oral dextromethorphan (a sensitive CYP2D6 substrate) increased the dextromethorphan AUC_{0-∞} by 144% and Cₘₐₓ by 70%;
(iv) Coadministration of rivoceranib 200 mg once daily with oral omeprazole (a sensitive CYP2C19 substrate) increased the omeprazole AUC_{0-∞} by 77% and Cₘₐₓ by 62%; and
(v) Coadministration of rivoceranib 200 mg once daily with oral caffeine (a sensitive CYP1A2 substrate) decreased the caffeine AUC_{0-∞} by 20% and Cₘₐₓ by 7%.

These results indicate the potential for a drug-drug interaction (DDI) to occur with a wide variety of small molecule therapeutics that is a substrate of any one or more of CYP3A4/A5, CYP2D6, CYP2C19, or CYP1A2 in patients concomitantly receiving administration of rivoceranib. Specifically, a substrate of any one or more of CYP3A4/A5, CYP2D6, or CYP2C19 has the potential to be cleared at a slower rate in the presence of rivoceranib because of inhibition of any one or more of these P450 enzymes, which represents a risk of over-exposure and potential adverse events (AEs) associated with the substrate. A substrate of CYP1A2 has the potential for an increased clearance rate when administered with rivoceranib because of induction of CYP1A2, which represents a risk of under-exposure and loss of efficacy associated with the substrate. The above risks associated with concomitant administration of rivoceranib with a substrate of any one of CYP3A4/A5, CYP2D6, CYP2C19, or CYP1A2 are addressed and managed by the methods of treatment described herein.

### Example 2

### Effect of Rivoceranib on the Pharmacokinetics of Cytochrome P450 Enzyme Substrates: A Phase 1 Trial in Healthy Volunteers

This example describes a pharmacokinetic interaction study in healthy human volunteers dosed with 700 mg rivoceranib and a mixture of cytochrome P450 enzyme (CYP1A2, CYP2C9, CYP2C19, CYP2D6, and CYP3A) substrates.

### Objectives

The primary objective of the study was to evaluate the effect of rivoceranib 700 mg once daily (QD) on the pharmacokinetics (PK) of various CYP substrates to determine drug-drug interactions of rivoceranib.

### Methodology

This was an open-label, 2-treatment, fixed-sequence drug-drug interaction phase 1 study designed to evaluated the impact of multiple oral doses of 700 mg rivoceranib on the single-dose PK of CYP enzyme substrates administered in a 5+1 Cooperstown probe cocktail (caffeine [CYP1A2], S- and R-warfarin [CYP2C9]+vitamin K, omeprazole [CYP2C19], dextromethorphan [CYP2D6], and midazolam [CYP3A]) in healthy volunteers (N=32). On day 1, volunteers received a single dose of the 5+1 cocktail comprising 200 mg caffeine, 10 mg S- and R-warfarin with 10 mg vitamin K, 40 mg omeprazole, 30 mg dextromethorphan, and 2 mg midazolam. Blood samples were collected pre-dose on day 1 and up to 120 hours post-dose for PK analyses of the substrates in the 5+1 cocktail. On days 6 to 15, volunteers received 700 mg rivoceranib once daily for 10 consecutive days with a single dose of the 5+1 cocktail administered on day 11. Blood samples were collected pre-dose on day 11 and up to 120 hours post cocktail dosing (day 16) for PK analyses. Each dosing was under fasted conditions. There was a washout period of 5 days between day 1 dosing and the first rivoceranib dose on day 6; the 2 cocktail dosings were spaced by 10 days. The overall clinical study design is shown in FIG. 12.

### Study Demographics

Baseline demographics of healthy volunteers appear in Table 13.

TABLE 13
Baseline demographics

| Characteristic | Safety Population |
|---|---|
| Age (years), mean (SD) | 43.5 (8.2) |
| Sex, n (%) | — |
| Female | 16 (50) |
| Male | 16 (50) |
| Race, n (%) | — |
| Black or African American | 3 (9) |
| White | 29 (91) |
| Ethnicity, n (%) | — |
| Hispanic or Latino | 25 (78) |
| Non Hispanic or Latino | 7 (22) |
| Body mass index (Kg/M³), mean (SD) | 27.4 (3.3) |
| Height (cm), mean (SD) | 167.5 (9.3) |
| Weight (Kg), mean (SD) | 77.1 (11.4) |

### Results

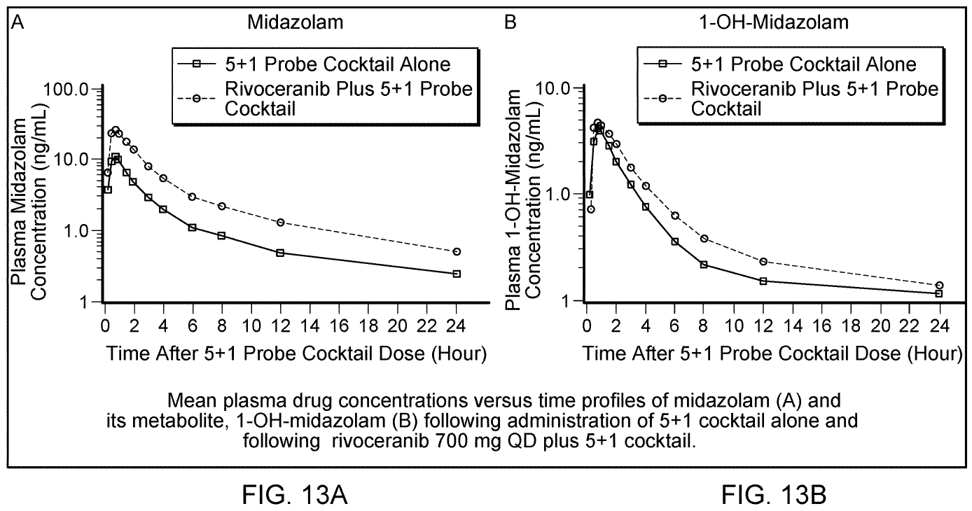

All cytochrome P-450 enzymes interrogated by the Cooperstown 5+1 cocktail were modulated by rivoceranib. The exposure results for Cooperstown 5+1 cocktail and Cooperstown 5+1 cocktail with coadministration with rivoceranib appear in FIGS. 13-17 and pharmacokinetics summarized in Tables 14-18.

Coadministration with rivoceranib 700 mg caused inhibition of the CYP3A subfamily (CYP3A4/A5), as evidenced by an increase of 2.4- to 2.8-fold in exposure of midazolam following coadministration with rivoceranib (see FIG. 13 and Table 14).

TABLE 14
Change in PK parameters for Midazolam and 1-hydroxymidazolam^{b}

| | 5 + 1 cocktail | | Rivoceranib plus | | Geometrical | |
|---|---|---|---|---|---|---|
| | alone | | 5 + 1 cocktail | | mean | Intra- |
| | Mean | Geometric | Mean | Geometric | Ratioª, % | participant |
| PK Parameter | (CV %) | LSM | (CV %) | LSM | (90% CI) | CV % |

Midazolam

| | | | | | | |
|---|---|---|---|---|---|---|
| AUC₍₀₋ₜ₎ | 28.09 | 28.09 | 75.46 | 75.17 | 267.59 | 30.37 |
| | (47.5) | | (36.8) | | (234.28- | |
| | [n = 32] | | [n = 28] | | 305.64) | |
| AUC_{(0-∞)} | 29.71 | 29.71 | 82.47 | 82.08 | 276.27 | 29.97 |
| | (48.7) | | (41.8) | | (242.21- | |
| | [n = 32] | | [n = 28] | | 315.120 | |
| Cₘₐₓ | 10.79 | 10.79 | 25.72 | 25.67 | 237.88 | 26.76 |
| (ng/mL) | (37.9) | | (32.9) | | (211.54- | |
| | [n = 32] | | [n = 28] | | 267.51) | |

1-hydroxymidazolam^{b}

| | | | | | | |
|---|---|---|---|---|---|---|
| AUC₍₀₋ₜ₎ | 9.643 | 9.643 | 14.37 | 13.89 | 144.05 | 16.13 |
| | (35.0) | | (33.8) | | (133.98- | |
| | [n = 32] | | [n = 28] | | 154.88) | |
| AUC_{(0-∞)} | 10.18 | 10.21 | 15.67 | 15.16 | 148.48 | 16.34 |
| | (35.4) | | (34.2) | | (137.80- | |
| | [n = 31] | | [n = 28] | | 159.99) | |
| Cₘₐₓ | 4.115 | 4.115 | 4.797 | 4.639 | 112.73 | 15.34 |
| (ng/mL) | (38.0) | | (30.7) | | (105.21- | |
| | [n = 32] | | [n = 28] | | 120.79) | |

^{a}Geometric Mean Ratio: 100* LSM of Rivoceranib Plus 5 + 1 Cocktail/LSM of 5 + 1 Cocktail Alone;
^{b}1-hydroxymidazolam is a metabolite of Midazolam

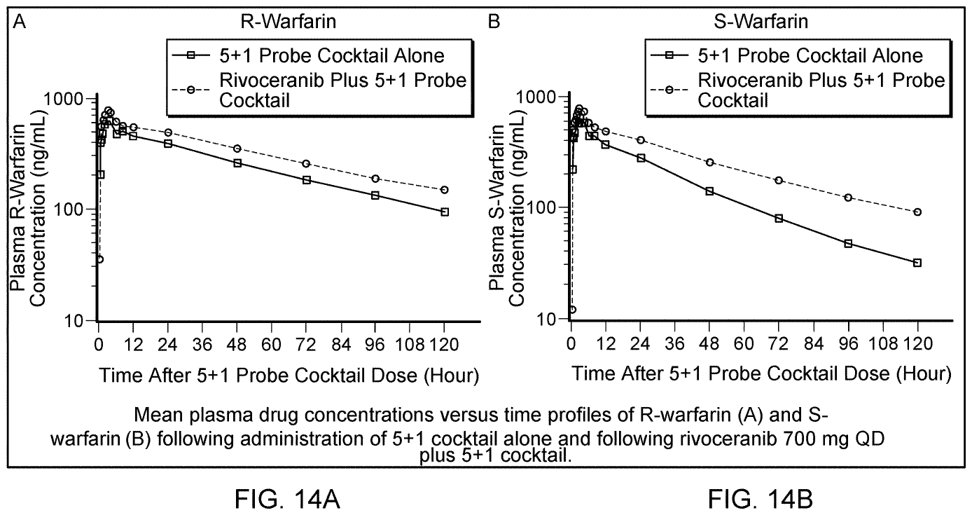

When co-administered with rivoceranib 700 mg. S-warfarin and R-warfarin AUC_{0-inf} increased by 68% and 32% and Cmax by 19% and 15%, respectively, indicating rivoceranib inhibits CYP2C9 (FIG. 14 and Table 15).

TABLE 15
Change in PK parameters for R-and S-warfarin

| | 5 + 1 cocktail | | Rivoceranib plus | | Geometrical | |
|---|---|---|---|---|---|---|
| | alone | | 5 + 1 cocktail | | mean | Intra- |
| | Mean | Geometric | Mean | Geometric | Ratioª, % | participant |
| PK Parameter | (CV %) | LSM | (CV %) | LSM | (90% CI) | CV % |

R-Warfarin

| | | | | | | |
|---|---|---|---|---|---|---|
| AUC₍₀₋ₜ₎ | 29,860 | 29,860 | 36,660 | 36,090 | 120.86 | 12.60 |
| | (21.9) | | (30.7) | | (113.69- | |
| | [n = 32] | | [n = 24] | | 128.48) | |
| AUC_{(0-∞)} | 36,750 | 36,750 | 49,220 | 48,560 | 132.12 | 15.24 |
| | (24.4) | | (38.5) | | (122.90- | |
| | [n = 32] | | [n = 25] | | 142.02) | |
| Cₘₐₓ | 701.2 | 701.2 | 816.0 | 804.9 | 114.79 | 14.13 |
| (ng/mL) | (24.7) | | (22.5) | | (107.75- | |
| | [n = 32] | | [n = 28] | | 122.29) | |

S-Warfarin

| | | | | | | |
|---|---|---|---|---|---|---|
| AUC₍₀₋ₜ₎ | 18,880 | 18.880 | 28,920 | 28,490 | 150.88 | 13.62 |
| | (19.2) | | (28.6) | | (141.30- | |
| | [n = 32] | | [n = 24] | | 161.10) | |
| AUC_{(0-∞)} | 20,640 | 20,640 | 34,780 | 34,670 | 167.95 | 16.25 |
| | (20.7) | | (33.4) | | (155.99- | |
| | [n = 32] | | [n = 27] | | 180.83) | |
| Cₘₐₓ | 701.1 | 701.1 | 840.2 | 832.5 | 118.74 | 16.03 |
| (ng/mL) | (24.9) | | (23.4) | | (110.54- | |
| | [n = 32] | | [n = 28] | | 127.54) | |

^{a}Geometric Mean Ratio: 100* LSM of Rivoceranib Plus 5 + 1 Cocktail/LSM of 5 + 1 Cocktail Alone

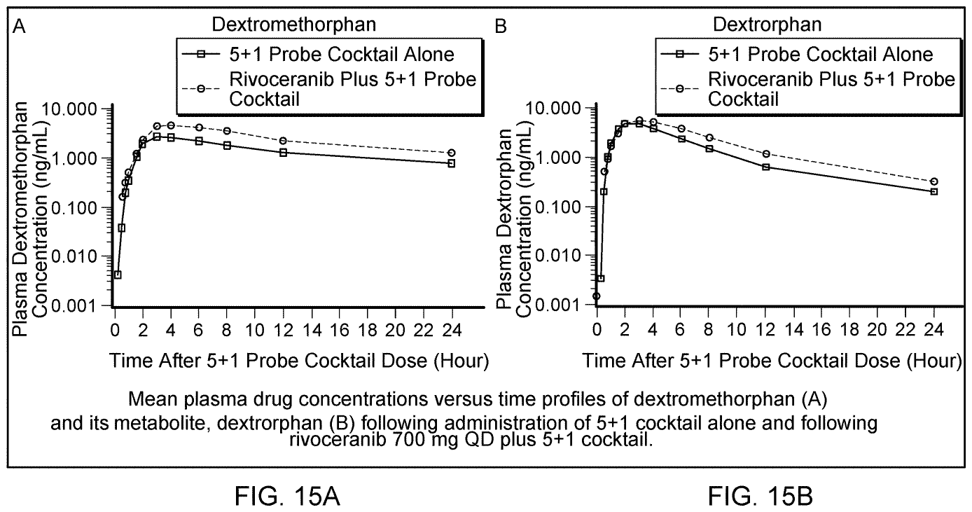

Dextromethorphan metabolism (CYP2D6) was inhibited by rivoceranib 700 mg, with a 2- to 2.7-fold increase in dextromethorphan exposure (FIG. 15 and Table 16).

TABLE 16
Change in PK parameters for Dextromethorphan and Dextrorphanb

| | 5 + 1 cocktail | | Rivoceranib plus | | Geometrical | |
|---|---|---|---|---|---|---|
| | alone | | 5 + 1 cocktail | | mean | Intra- |
| | Mean | Geometric | Mean | Geometric | Ratioª, % | participant |
| PK Parameter | (CV %) | LSM | (CV %) | LSM | (90% CI) | CV % |

Dextromethorphan

| | | | | | | |
|---|---|---|---|---|---|---|
| AUC₍₀₋ₜ₎ | 10.43 | 10.43 | 23.66 | 27.11 | 259.87 | 51.87 |
| | (241.2) | | (217.3) | | (208.26- | |
| | [n = 32] | | [n = 28] | | 324.27) | |
| AUC_{(0-∞)} | 13.97 | 13.30 | 33.24 | 36.24 | 272.50 | 41.59 |
| | (196.0) | | (173.1) | | (225.72- | |
| | [n = 31] | | [n = 27] | | 328.96) | |
| Cₘₐₓ | 1.397 | 1.397 | 2.527 | 2.778 | 198.80 | 45.45 |
| (ng/mL) | (166.4) | | (165.4) | | (163.35- | |
| | [n = 32] | | [n = 28] | | 241.95) | |

Dextrorphan^{b}

| | | | | | | |
|---|---|---|---|---|---|---|
| AUC₍₀₋ₜ₎ | 26.14 | 26.14 | 34.50 | 33.74 | 129.07 | 25.62 |
| | (61.6) | | (85.3) | | (115.14- | |
| | [n = 32] | | [n = 28] | | 144.68 | |
| AUC_{(0-∞)} | 27.85 | 27.85 | 37.84 | 37.10 | 133.22 | 21.75 |
| | (57.9) | | (75.3) | | (120.85- | |
| | [n = 32] | | [n = 28] | | 146.85) | |
| Cₘₐₓ | 4.657 | 4.657 | 4.875 | 4.736 | 101.70 | 27.16 |
| (ng/mL) | (58.5) | | (72.4) | | (90.14- | |
| | [n = 32] | | [n = 28] | | 114.74) | |

^{a}Geometric Mean Ratio: 100* LSM of Rivoceranib Plus 5 + 1 Cocktail/LSM of 5 + 1 Cocktail Alone
^{b}Dextrorphan is a metabolite of dextromethorphan

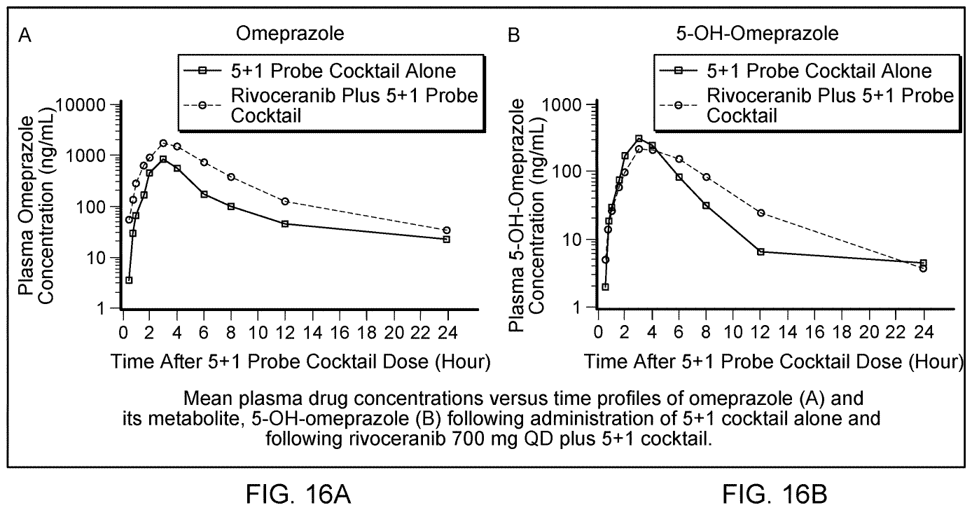

Rivoceranib 700 mg, appeared to act as an inhibitor of CYP2C19, increasing omeprazole AUC_{0-inf} 3.3-fold and increasing Cₘₐₓ 2-fold (FIG. 16 and Table 17).

TABLE 17
Change in PK parameters for Omeprazole and its metabolite 5-hydroxyomeprazole

| | 5 + 1 cocktai | | Rivoceranib plus | | Geometrical | |
|---|---|---|---|---|---|---|
| | l alone | | 5 + 1 cocktail | | mean | Intra- |
| | Mean | Geometric | Mean | Geometric | Ratioª, % | participant |
| PK Parameter | (CV %) | LSM | (CV %) | LSM | (90% CI) | CV % |

Omeprazole

| | | | | | | |
|---|---|---|---|---|---|---|
| AUC₍₀₋ₜ₎ | 10.43 | 10.43 | 23.66 | 27.11 | 259.87 | 51.87 |
| | (241.2) | | (217.3) | | (208.26- | |
| | [n = 32] | | [n = 28] | | 324.27) | |
| AUC_{(0-∞)} | 13.97 | 13.30 | 33.24 | 36.24 | 272.50 | 41.59 |
| | (196.0) | | (173.1) | | (225.72- | |
| | [n = 31] | | [n = 27] | | 328.96) | |
| Cₘₐₓ | 1.397 | 1.397 | 2.527 | 2.778 | 198.80 | 45.45 |
| (ng/mL) | (166.4) | | (165.4) | | (163.35 | |
| | [n = 32] | | [n = 28] | | 241.95) | |

5-hydroxyomeprazole^{b}

| | | | | | | |
|---|---|---|---|---|---|---|
| AUC₍₀₋ₜ₎ | 26.14 | 26.14 | 34.50 | 33.74 | 129.07 | 25.62 |
| | (61.6) | | (85.3) | | (115.14- | |
| | [n = 32] | | [n = 28] | | 144.68 | |
| AUC_{(0-∞)} | 27.85 | 27.85 | 37.84 | 37.10 | 133.22 | 21.75 |
| | (57.9) | | (75.3) | | (120.85- | |
| | [n = 32] | | [n = 28] | | 146.85) | |
| Cₘₐₓ | 4.657 | 4.657 | 4.875 | 4.736 | 101.70 | 27.16 |
| (ng/mL) | (58.5) | | (72.4) | | (90.14- | |
| | [n = 32] | | [n = 28] | | 114.74) | |

^{a}Geometric Mean Ratio: 100* LSM of Rivoceranib Plus 5 + 1 Cocktail/LSM of 5 + 1 Cocktail Alone
^{b}5-hydroxyomeprazole is a metabolite of Omeprazole

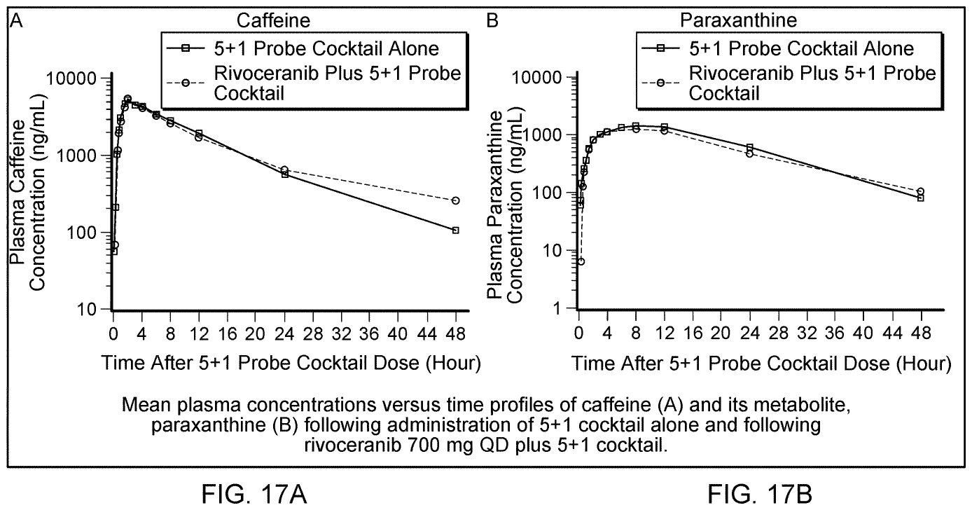

Rivoceranib 700 mg had a weak inductive effect on CYP1A2, reducing caffeine AUC_{0-inf} by 15%, while leaving the Cₘₐₓ of caffeine relatively unchanged (FIG. 17 and Table 18).

TABLE 18
Change in PK parameters for Caffeine and its metabolite Paraxanthine

| | 5 + 1 cocktail | | Rivoceranib plus | | Geometrical | |
|---|---|---|---|---|---|---|
| | alone | | 5 + 1 cocktail | | mean | Intra- |
| | Mean | Geometric | Mean | Geometric | Ratio^{a}, % | participant |
| PK Parameter | (CV %) | LSM | (CV %) | LSM | (90% CI) | CV % |

Caffeine

| | | | | | | |
|---|---|---|---|---|---|---|
| AUC₍₀₋ₜ₎ | 52,630 | 52,630 | 44,480 | 43,190 | 43,190 | 19.31 |
| | (49.9) | | (74.7) | | | |
| | [n = 32] | | [n = 24] | | | |
| AUC_{(0-∞)} | 53,440 | 53,440 | 47,620 | 45,360 | 84.87 | 19.03 |
| | (50.9) | | (72.6) | | (77.91- | |
| | [n = 32] | | [n = 28] | | 92.46) | |
| Cₘₐₓ | 5,193 | 5,193 | 5,329 | 5,214 | 100.41 | 15.75 |
| (ng/mL) | | | (36.5) | | (93.55 | |
| | | | [n = 28] | | 107.77) | |

Paraxanthine^{b}

| | | | | | | |
|---|---|---|---|---|---|---|
| AUC₍₀₋ₜ₎ | 30,950 | 30,950 | 26,400 | 26,750 | 86.42 | 12.62 |
| | (28.9) | | (38.2) | | (81.26- | |
| | [n = 32] | | [n = 24] | | 91.91) | |
| AUC_{(0-∞)} | 28,730 | 31,720 | 25,040 | 24,990 | 78.80 | 10.25 |
| | (21.7) | | (44.0) | | (72.22- | |
| | [n = 16] | | [n = 15] | | 85.98) | |
| Cₘₐₓ | 1,436 | 1,436 | 1,430 | 1,429 | 99.49 | 13.93 |
| (ng/mL) | (23.9) | | (21.4) | | (93.48- | |
| | [n = 32] | | [n = 28] | | 105.90) | |

^{a}Geometric Mean Ratio: 100* LSM of Rivoceranib Plus 5 + 1 Cocktail/LSM of 5 + 1 Cocktail Alone
^{b}Paraxanthine is a metabolite of Caffeine

### Clinical Safety

Among the 26 of 32 (81.3%) volunteers who experienced treatment emergent adverse events (TEAFs), 12 (37.5%) had a TEAE after 5+1 cocktail alone, 16 (53.30%) after rivoceranib alone, and 20 (71.4%) after rivoceranib plus 5+1 cocktail. There were no serious (grade 4) TEAEs. Eight participants discontinued the study due to TEAEs (including 7 due to grade 3 increased blood pressure) and 1 additional participant experienced TEAEs (chest discomfort, coughing, and fatigue) that led to dosing interruption and discontinuation from the study. The TEAEs reported by 4 participants were headache (62.5% of participants), constipation (34.4%), increased blood pressure (31.1%), back pain (18.8%), diarrhea (12.5%), and photophobia (12.5%).

### Results Summary

From the results presented in Example 2 it can be seen that rivoceranib at a dosage of 700 mg QD reduced caffeine AUC_{0-inf} by 15%, but did not change caffeine Cₘₐₓ, indicating a weak inductive effect of rivoceranib on the PK of CYP1A2 substrates. When co-administered with rivoceranib, S-warfarin and R-warfarin AUC_{0-inf} increased by 68% and 32%, and Cₘₐₓ by 19% and 15%, respectively, indicating rivoceranib inhibits CYP2C9. Rivoceranib appeared to act as an inhibitor of CYP2C19, increasing omeprazole AUC_{0-inf} 3.3-fold and increasing Cₘₐₓ 2-fold. Dextromethorphan metabolism (CYP2D6) was inhibited, with a 2- to 2.7-fold increase in dextromethorphan exposure. Rivoceranib appeared to inhibit midazolam metabolism by CYP3A4, with 2.4- to 2.8-fold increases in midazolam exposures.

The effect of rivoceranib on the PK of CYP1A2 substrates is less clinically significant than its effects on PK of other CYP substrates. Rivoceranib inhibits the metabolism of CYP2C9, CYP2C19, CYP2D6, and CYP3A4 substrates, suggesting that dose adjustment of substrates of these CYP isozymes and/or cautiously monitoring patients' adverse events may be needed when they are co-administered with rivoceranib.

### OTHER EMBODIMENTS

While various embodiments of the present disclosure have been shown and described herein, such embodiments are provided by way of example only. Numerous variations, changes, and substitutions will now occur to those skilled in the art without departing from the inventions recited in the following claims. It should be understood that various alternatives to the embodiments of the disclosure can be employed in practice. It is intended that the following claims define the scope of the invention and that methods and structures within the scope of the claims and their equivalents be covered thereby.

1. (canceled)

2. A method of treating cancer, the method comprising:
(a) determining if a patient in need of a cancer therapy is receiving administration of a substrate of cytochrome P-450 (CYP) selected from any one or more of CYP3A4/A5, CYP2D6, CYP2C19, or CYP1A2;
(b) administering to the patient a therapeutically effective amount of rivoceranib; and
(c) either
(i) discontinuing administration of the CYP substrate prior to and during administering the rivoceranib, or
(ii) adjusting a daily dosage of the CYP substrate and concomitantly administering the rivoceranib.

3. The method of treating cancer of claim 2, wherein
administration of the CYP substrate is discontinued for a time period of about 5 or more half-lives of the substrate before
administering to the patient a therapeutically effective amount of rivoceranib.

4. The method of treating cancer of claim 2, wherein the adjusted daily dosage of the CYP substrate is reduced to about 25% to about 95% of an intended daily dosage of the CYP substrate if the patient were not receiving concomitant rivoceranib.

5. The method of treating cancer of claim 2, further comprising any one or more of the following:
(i) advising the patient that the concomitant administration of rivoceranib and the substrate can alter a therapeutic effect of the substrate; and
(ii) advising the patient that the substrate should be used with caution in patients receiving rivoceranib due to a potential change in pharmacokinetics of the substrate.

6. The method of claim 2, wherein the CYP substrate is dextromethorphan.

7. The method of claim 2, wherein the CYP is CYP3A4/A5.

8. The method of claim 2, wherein the CYP is CYP2D6.

9. The method of claim 2, wherein the CYP is CYP2C9.

10. The method of claim 2, wherein the CYP is CYP2C19.

11. The method of claim 2, wherein the CYP is CYP1A2.

12. The method of claim 2, wherein the cancer is Acanthoma, Acinic cell carcinoma, Acoustic neuroma, Acral lentiginous melanoma, Acrospiroma, Acute eosinophilic leukemia, Acute lymphoblastic leukemia, Acute megakaryoblastic leukemia, Acute monocytic leukemia, Acute myeloblastic leukemia with maturation, Acute myeloid dendritic cell leukemia, Acute myeloid leukemia, Acute promyelocytic leukemia, Adamantinoma, Adenocarcinoma, Adenoid cystic carcinoma, Adenoma, Adenomatoid odontogenic tumor, Adrenocortical carcinoma, Adult T-cell leukemia, Aggressive NK-cell leukemia, AIDS-Related Cancers, AIDS-related lymphoma, Alveolar soft part sarcoma, Ameloblastic fibroma, Anal cancer, Anaplastic large cell lymphoma, Anaplastic thyroid cancer, Angioimmunoblastic T-cell lymphoma, Angiomyolipoma, Angiosarcoma, Appendix cancer, Astrocytoma, Atypical teratoid rhabdoid tumor, Basal cell carcinoma, Basal-like carcinoma, B-cell leukemia, B-cell lymphoma, Bellini duct carcinoma, Biliary tract cancer, Bladder cancer, Blastoma, Bone Cancer, Bone tumor, Brain Stem Glioma, Brain Tumor, Breast Cancer, Brenner tumor, Bronchial Tumor, Bronchioloalveolar carcinoma, Brown tumor, Burkitt's lymphoma, Cancer of Unknown Primary Site, Carcinoid Tumor, Carcinoma, Carcinoma in situ, Carcinoma of the penis, Carcinoma of Unknown Primary Site, Carcinosarcoma, Castleman's Disease, Central Nervous System Embryonal Tumor, Cerebellar Astrocytoma, Cerebral Astrocytoma, Cervical Cancer, Cholangiocarcinoma, Chondroma, Chondrosarcoma, Chordoma, Choriocarcinoma, Choroid plexus papilloma, Chronic Lymphocytic Leukemia, Chronic monocytic leukemia, Chronic myelogenous leukemia, Chronic Myeloproliferative Disorder, Chronic neutrophilic leukemia, Clear-cell tumor, Colon Cancer, Colorectal cancer, Craniopharyngioma, Cutaneous T-cell lymphoma, Degos disease, Dermatofibrosarcoma protuberans, Dermoid cyst, Desmoplastic small round cell tumor, Diffuse large B cell lymphoma, Dysembryoplastic neuroepithelial tumor, Embryonal carcinoma, Endodermal sinus tumor, Endometrial cancer, Endometrial Uterine Cancer, Endometrioid tumor, Enteropathy-associated T-cell lymphoma, Ependymoblastoma, Ependymoma, Epithelioid sarcoma, Erythroleukemia, Esophageal cancer, Esthesioneuroblastoma, Ewing's sarcoma, Extracranial Germ Cell Tumor, Extragonadal Germ Cell Tumor, Extrahepatic Bile Duct Cancer, Extramammary Paget's disease, Fallopian tube cancer, Fetus in fetu, Fibroma, Fibrosarcoma, Follicular lymphoma, Follicular thyroid cancer, Gallbladder Cancer, Ganglioglioma, Ganglioneuroma, Gastric Cancer, Gastric lymphoma, Gastrointestinal cancer, Gastrointestinal Carcinoid Tumor, Gastrointestinal Stromal Tumor, Gastrointestinal stromal tumor, Germ cell tumor, Germinoma, Gestational choriocarcinoma, Gestational Trophoblastic Tumor, Giant cell tumor of bone, Glioblastoma multiforme, Glioma, Gliomatosis cerebri, Glomus tumor, Glucagonoma, Gonadoblastoma, Granulosa cell tumor, Hairy Cell Leukemia, Hairy cell leukemia, Head and Neck Cancer, Heart cancer, Hemangioblastoma, Hemangiopericytoma, Hemangiosarcoma, Hematological malignancy, Hepatocellular carcinoma, Hepatosplenic T-cell lymphoma, Hereditary breast-ovarian cancer syndrome, Hodgkin's lymphoma, Hypopharyngeal Cancer, Hypothalamic Glioma, Inflammatory breast cancer, Intraocular Melanoma, Islet cell carcinoma, Islet Cell Tumor, Juvenile myelomonocytic leukemia, Kaposi Sarcoma, Kaposi's sarcoma, Kidney Cancer, Klatskin tumor, Krukenberg tumor, Laryngeal Cancer, Laryngeal cancer, Lentigo maligna melanoma, Leukemia, Lip and Oral Cavity Cancer, Liposarcoma, Lung cancer, Luteoma, Lymphangioma, Lymphangiosarcoma, Lymphoepithelioma, Lymphoid leukemia, Lymphoma, Macroglobulinemia, Malignant Fibrous Histiocytoma, Malignant Fibrous Histiocytoma of Bone, Malignant Glioma, Malignant Mesothelioma, Malignant peripheral nerve sheath tumor, Malignant rhabdoid tumor, Malignant triton tumor, MALT lymphoma, Mantle cell lymphoma, Mast cell leukemia, Mediastinal germ cell tumor, Mediastinal tumor, Medullary thyroid cancer, Medulloblastoma, Medulloepithelioma, Melanoma, Meningioma, Merkel Cell Carcinoma, Mesothelioma, Mesothelioma, Metastatic Squamous Neck Cancer with Occult Primary, Metastatic urothelial carcinoma, Mixed Mullerian tumor, Monocytic leukemia, Mouth Cancer, Mucinous tumor, Multiple Endocrine Neoplasia Syndrome, Multiple Myeloma, Mycosis fungoides, Myelodysplastic Syndromes, Myeloid leukemia, Myeloid sarcoma, Myeloproliferative Disease, Myxoma, Nasal Cavity Cancer, Nasopharyngeal Cancer, Neoplasm, Neurinoma, Neuroblastoma, Neurofibroma, Neuroma, Nodular melanoma, Non-Hodgkin's Lymphoma, Non-melanoma Skin Cancer, Non-Small Cell Lung Cancer, Ocular oncology, Oligoastrocytoma, Oligodendroglioma, Oncocytoma, Optic nerve sheath meningioma, Oral Cancer, Oral cancer, Oropharyngeal Cancer, Osteosarcoma, Ovarian Cancer, Ovarian Epithelial Cancer, Ovarian Germ Cell Tumor, Ovarian Low Malignant Potential Tumor, Paget's disease of the breast, Pancoast tumor, Pancreatic cancer, Papillary thyroid cancer, Papillomatosis, Paraganglioma, Paranasal Sinus Cancer, Parathyroid Cancer, Penile Cancer, Perivascular epithelioid cell tumor, Pharyngeal Cancer, Pheochromocytoma, Pineal Parenchymal Tumor of Intermediate Differentiation, Pineoblastoma, Pituicytoma, Pituitary adenoma, Pituitary tumor, Plasma Cell Neoplasm, Pleuropulmonary blastoma, Polyembryoma, Precursor T-lymphoblastic lymphoma, Primary central nervous system lymphoma, Primary effusion lymphoma, Primary Hepatocellular Cancer, Primary Liver Cancer, Primary peritoneal cancer, Primitive neuroectodermal tumor, Prostate cancer, Pseudomyxoma peritonei, Rectal Cancer, Renal cell carcinoma, Respiratory Tract Carcinoma Involving the NUT Gene on Chromosome 15, Retinoblastoma, Rhabdomyoma, Rhabdomyosarcoma, Richter's transformation, Sacrococcygeal teratoma, Salivary Gland Cancer, Sarcoma, Schwannomatosis, Sebaceous gland carcinoma, Secondary neoplasm, Seminoma, Serous tumor, Sertoli-Leydig cell tumor, Sex cord-stromal tumor, Sezary Syndrome, Signet ring cell carcinoma, Skin Cancer, Small blue round cell tumor, Small cell carcinoma, Small Cell Lung Cancer, Small cell lymphoma, Small intestine cancer, Soft tissue sarcoma, Somatostatinoma, Soot wart, Spinal Cord Tumor, Spinal tumor, Splenic marginal zone lymphoma, Squamous cell carcinoma, Stomach cancer, Superficial spreading melanoma, Supratentorial Primitive Neuroectodermal Tumor, Surface epithelial-stromal tumor, Synovial sarcoma, T-cell acute lymphoblastic leukemia, T-cell large granular lymphocyte leukemia, T cell leukemia, T-cell lymphoma, T-cell prolymphocytic leukemia, Teratoma, Terminal lymphatic cancer, Testicular cancer, Thecoma, Throat Cancer, Thymic Carcinoma, Thymoma, Thyroid cancer, Transitional Cell Cancer of Renal Pelvis and Ureter, Transitional cell carcinoma, Urachal cancer, Urethral cancer, Urogenital neoplasm, Uterine sarcoma, Uveal melanoma, Vaginal Cancer, Verner Morrison syndrome, Verrucous carcinoma, Visual Pathway Glioma, Vulvar Cancer, Waldenstrom's macroglobulinemia, Warthin's tumor, Wilms' tumor, or any combination thereof.

13. The method of claim 12, wherein the cancer is Adenoid Cystic Carcinoma.

14. The method of claim 12, wherein the cancer is Hepatocellular Carcinoma.

15. The method of claim 2, wherein the therapeutically effective amount of rivoceranib or a pharmaceutically acceptable salt thereof is about 100 mg to about 900 mg.

16. The method of claim 15, wherein the therapeutically effective amount of rivoceranib or a pharmaceutically acceptable salt thereof is about 100 mg to about 800 mg.

17. The method of claim 15, wherein the therapeutically effective amount of rivoceranib or a pharmaceutically acceptable salt thereof is about 200 mg to about 750 mg.

18. The method of claim 15, wherein the therapeutically effective amount of rivoceranib or a pharmaceutically acceptable salt thereof is about 300 mg to about 600 mg.

19. The method of claim 15, wherein the therapeutically effective amount of rivoceranib or a pharmaceutically acceptable salt thereof is about 400 mg to about 500 mg.

20-22. (canceled)
